(19) United States
(12) Patent Application Publication
(10) Pub. No.: US 2023/0132806 A1
(43) Pub. Date: May 4, 2023

(54) Systems and Methods for Control of Refractive Index and Optical Properties in Living Biological Cells

(71) Applicant: The Regents of the University of California, Oakland, CA (US)

(72) Inventors: Alon A. Gorodetsky, Irvine, CA (US); Atrouli Chatterjee, Irvine, CA (US); Juana Alejandra Cerna Sanchez, Irvine, CA (US)

(73) Assignee: The Regents of the University of California, Oakland, CA (US)

(21) Appl. No.: 17/918,108

(22) PCT Filed: Apr. 12, 2021
(86) PCT No.: PCT/US2021/026920

§ 371 Date: Oct. 10, 2022

Related U.S. Application Data
(60) Provisional application No. 63/008,474, filed on Apr. 10, 2020.

(51) Int. Cl. C12N 15/85

(52) U.S. Cl. CPC C12N 15/85; C12N 2800/107; C12N 2830/002

(57) ABSTRACT

The description of living biological cells comprising heterologously expressed reflectin biomolecules that can be used to dynamically tune the optical properties of the host cells, as well as of the methods of fabrication thereof, are provided. Methods of regulating optical properties, including local refractive indices, of such cells with external stimuli are also provided.

### CROSS REFERENCE TO RELATED APPLICATIONS

This application claims priority to U.S. Provisional Application Ser. No. 63/008,474, entitled “Systems and Methods for Control of Refractive Index and Optical Properties in Living Biological Cells” to Gorodetsky et al., filed Apr. 10, 2020; the disclosure of which is incorporated herein by reference in its entirety.

### STATEMENT REGARDING FEDERALLY SPONSORED RESEARCH

This invention was made with Government support under Grant No. W911NF-17-2-0142 (Cooperative Agreement) awarded by the Defense Advanced Research Projects Agency, Grant No. FA9550-17-1-0024 and Grant No. FA9550-16-1-0296 awarded by the Air Force Office of Scientific Research. The Government has certain rights in this invention.

### FIELD OF THE INVENTION

The invention is generally directed to systems and methods to control refractive index of living cells, including mammalian cells, cell components, and biomolecules, as well as of organs or whole organisms containing such cells, more specifically, towards engineered biological systems that incorporate cephalopod reflectin and possess tunable optical properties.

### BACKGROUND OF THE INVENTION

The idea of humans vanishing from sight by becoming transparent or invisible has captured the imagination of the general populace and scientists alike for millennia. These concepts have been extensively explored in classic literature, including Plato describing the hypothetical Ring of Gyges—an item that allowed its wearer to disappear (see Plato & Jowett, B. (Transl.) The Republic, Dover Publications, Inc., U.S.A, 2000, the disclosure of which is incorporated herein by reference), and H. G. Wells envisioning that a scientist could match his refractive index to that of air to become invisible (see Wells, H. G. The Invisible Man, Penguin Group, New York, 2005, the disclosure of which is incorporated herein by reference). While such ideas may initially seem fantastic, the natural world is filled with examples of animals, such as the glasswing butterfly (see Siddique, R. H., et al. The role of random nanostructures for the omnidirectional anti-reflection properties of the glasswing butterfly. Nat. Comm. 6, 2015, the disclosure of which is incorporated herein by reference), the grass shrimp (see Bhandiwad, A. & Johnsen, S. The effects of salinity and temperature on the transparency of the grass shrimp Palaemonetes pugio. J. Exp. Biol. 214, 709-716, 2011, the disclosure of which is incorporated herein by reference), the comb jellyfish (see Welch, V., et al. Optical properties of the iridescent organ of the comb-jellyfish Beroe Cucumis (Ctenophora). Phys. Rev. E 73, 2006, the disclosure of which is incorporated herein by reference), the glass frog (see Guayasamin, J. M. et al. A marvelous new glassfrog (Centrolenidae, Hyalinobatrachium) from Amazonian Ecuador. Zookeys. 673, 1-20, 2017, the disclosure of which is incorporated herein by reference), and mesopelagic cephalopods (see Zylinski, S. & Johnsen, S. Mesopelagic cephalopods switch between transparency and pigmentation to optimize camouflage in the deep. Curr. Biol. 21, 1937-1941, 2011, the disclosure of which is incorporated herein by reference) that have evolved transparent structures, tissues, and even whole bodies for the purpose of concealment and as highly-effective forms of camouflage (see Johnsen, S. Hidden in plain sight: the ecology and physiology of organismal transparency. The Biological Bulletin 201, 301-318, 2001; and Johnsen, S. Hide and seek in the open sea: pelagic camouflage and visual countermeasures. Annual review of marine science 6, 369-392, 2014, the disclosures of which are incorporated herein by reference). From a technological perspective, the study of transparency (wherein transparency is defined as the property of transmitting light without appreciable scattering, so that objects lying beyond are seen clearly) has recently attracted significant attention. This renewed interest in transparency has been motivated by the emergence of laboratory techniques (such as tissue clearing methods) for making deceased mammalian tissues and organs partially transparent and, thus, amenable to three-dimensional visualization. This work has helped answer outstanding fundamental questions in biology and medicine (see Richardson, D. S., et al. Clarifying tissue clearing. Cell 162, 246-257, 2015; and Susaki, E. A., et al. Whole-body and whole-organ clearing and imaging techniques with single-cell resolution: toward organism-level systems biology in mammals. Cell Chem. Biol. 23, 137-157, 2016, the disclosures of which are incorporated herein by reference). In this regard, whether in nature or in the laboratory, the transparency of biological systems has typically been achieved in the same way—by maximizing the direct transmission of visible light, while simultaneously minimizing competing optical processes, such as the absorption of light by biomolecules found in the system of interest and, most importantly, the scattering of incident light due to differences in refractive index along its path.

### REFERENCE TO A SEQUENCE LISTING SUBMITTED ELECTRONICALLY VIA EFS-WEB

The instant application contains a Sequence Listing which has been filed electronically in ASCII format and is hereby incorporated by reference in its entirety. Said ASCII copy, created on Apr. 12, 2021, is named “06206PCT_Seq_List_ST25.txt” and is 97,686 bytes in size.

### SUMMARY OF THE INVENTION

Systems and methods for control of refractive index and optical properties in living biological cells are disclosed.

In one embodiment, a living biological cell includes a first cellular component characterized by a first non-native reflectin biomolecule.

In a further embodiment, the first reflectin biomolecule is selected from: a natural isoform of reflectin protein, a non-natural variant of reflectin protein, a non-natural biomolecule derived from or mimicking reflectin protein, and any combination thereof.

In another embodiment, the first cellular component is characterized by a tunable refractive index due to the first non-native reflectin biomolecule.

In a still further embodiment, the first cellular component is characterized by a tunable optical property due to the first non-native reflectin biomolecule.

In still another embodiment, the living biological cell further includes a second cellular component characterized by a second non-native reflectin biomolecule.

In a yet further embodiment, the second reflectin biomolecule is selected from: a natural isoform of reflectin protein, a non-natural variant of reflectin protein, a non-natural biomolecule derived from or mimicking reflectin protein, and any combination thereof.

In yet another embodiment, the second cellular component is characterized by a tunable refractive index due to the second non-native reflectin biomolecule.

In a further embodiment again, the second cellular component is characterized by a tunable optical property due to the second non-native reflectin biomolecule.

In another embodiment again, the living biological cell is of a cell type selected from: a bacterial cell, an archaeal cell, a plant cell, an animal cell and a fungal cell.

In a further additional embodiment, the living biological cell is a mammalian cell.

In another additional embodiment, the first cell component or second cell component is selected from: an organelle, a protein, a membrane, a cytoskeleton, and a ribosome.

In a still yet further embodiment, the tunable optical property is selected from: transmittance, reflectance, absorptance, and any combination thereof.

In still yet another embodiment, the tunable optical property is selected from: transparency, opaqueness, coloration, iridescence, and any combination thereof.

In a still further embodiment again, the first reflectin biomolecule or second reflectin biomolecule is the natural isoform of reflectin protein selected from: reflectin A1, reflectin A2, reflectin B1, reflectin C1, and another isoform or homologue; and any truncated or augmented version thereof.

In still another embodiment again, the living biological cell is a mammalian cell and the first reflectin biomolecule is reflectin A1.

In a still further additional embodiment, the tunable refractive index is tunable by application of an external stimulus selected from: ionic strength of the environment of the living biological cell, aromatic compounds, chemical, biological, biophysical, electrical, and mechanical stimulus, where the stimulus affects conformation and aggregation of reflectin biomolecule and or any of reflectin protein's isoforms, and any combination thereof.

In still another additional embodiment, the tunable refractive index is tunable by application of NaCl or acetylcholine.

In a yet further embodiment again, the tunable optical property is tunable by application of an external stimulus selected from: ionic strength of the environment of the living biological cell, aromatic compounds, chemical, biological, biophysical, electrical, and mechanical stimulus, where the stimulus affects conformation and aggregation of reflectin biomolecule and or any of reflectin protein's isoforms, and any combination thereof.

In yet another embodiment again, the tunable optical property is tunable by application of NaCl or acetylcholine.

In a yet further additional embodiment, the first non-native reflectin biomolecule or second non-native reflectin biomolecule form a subcellular photonic architecture characterized by an architecture shape and an architecture size.

In yet another additional embodiment, the architecture shape is a shape selected from: spheroid, platelet, microfiber, hexagonal plate, film, and any other fundamental geometric shape.

In a further additional embodiment again, the architecture size is in a range from nanometers to tens of microns.

In another additional embodiment again, the architectural size is in the range of approximately 5 nm to 5 μm.

In a still yet further embodiment again, the architecture shape and the architecture size are tunable by application of an external stimulus selected from: ionic strength of the environment of the living biological cell, aromatic compounds, chemical, biological, biophysical, electrical, and mechanical stimulus, where the stimulus affects conformation and aggregation of reflectin biomolecule and or any of reflectin protein's isoforms, and any combination thereof.

In still yet another embodiment again, the architecture shape and the architecture size are tunable by application of NaCl or acetylcholine.

In a still yet further additional embodiment, the tunable refractive index is adjustable within the range of ˜1.40 to ˜1.62.

In still yet another additional embodiment, a living biological system includes a plurality of living biological cells, where each living biological cell in the plurality of living biological cells comprises a cellular component characterized by a non-native reflectin biomolecule.

In a yet further additional embodiment again, the plurality of living biological cells are a cell type selected from: bacterial cells, archaeal cells, plant cells, animal cells, and fungal cells.

In yet another additional embodiment again, the plurality of living biological cells are mammalian cells.

In a still yet further additional embodiment again, wherein the cellular component is characterized by a tunable refractive index due to the reflectin biomolecule.

In still yet another additional embodiment again, the tunable refractive index is tunable by application of an external stimulus selected from: ionic strength of the environment of the living biological cell, aromatic compounds, chemical, biological, biophysical, electrical, and mechanical stimulus, where the stimulus affects conformation and aggregation of reflectin biomolecule and or any of reflectin protein's isoforms, and any combination thereof.

In another further embodiment, the tunable refractive index is tunable by application of NaCl or acetylcholine.

In still another further embodiment, the tunable refractive index is adjustable within the range of ˜1.40 to ˜1.62.

In yet another further embodiment, the cellular component is characterized by a tunable optical property due to the reflectin biomolecule.

In another further embodiment again, the tunable optical property is tunable by application of an external stimulus selected from: ionic strength of the environment of the living biological cell, aromatic compounds, chemical, biological, biophysical, electrical, and mechanical stimulus, where the stimulus affects conformation and aggregation of reflectin biomolecule and or any of reflectin protein's isoforms, and any combination thereof.

Another further additional embodiment, the tunable optical property is tunable by application of NaCl or acetylcholine.

In yet another further additional embodiment, the living biological system is a system selected from: an organ, a tissue, an organism.

In a further additional embodiment yet again, a method of controlling a refractive index and optical properties of a living cell and subcellular components includes providing a living biological cell, where the living biological cell includes various cell components and a plurality of reflectin biomolecules, where the reflectin biomolecule is a biomolecule selected from: a natural isoform of reflectin protein, a non-natural variant of reflectin protein, a non-natural biomolecule derived from or mimicking reflectin protein, and any combination thereof, where the living biological cell and the various cell components are each individually characterized by a tunable refractive index and tunable optical properties, and applying to the living biological cell an external stimulus capable of affecting the conformation or aggregation of the reflectin biomolecule to affect the tunable refractive index and the tunable optical properties of the plurality of reflectin biomolecules and of their immediate surrounding.

In a yet again another additional embodiment, a method includes transfecting a living biological cell with a plasmid encoding for expression of a reflectin biomolecule, where the reflectin biomolecule is a biomolecule selected from: a natural isoform of reflectin protein, a non-natural variant of reflectin protein, a non-natural biomolecule derived from or mimicking reflectin protein, and any combination thereof; and where the living biological cell already comprises various cell components prior to transfection; where the living cell and its various cell components become individually characterized by a tunable refractive index and tunable optical properties.

In another additional embodiment, the tunable refractive index and the tunable optical properties are adjusted via one or more of the choices selected from: choice of the plasmid, choice of the reflectin biomolecule, the expression level of the reflectin biomolecule, and any combination thereof.

### BRIEF DESCRIPTION OF THE DRAWINGS

These and other features and advantages of the present invention will be better understood by reference to the following detailed description when considered in conjunction with the accompanying data and figures, wherein:

FIGS. 1A through 1C illustrate leucophore-enabled adaptive transparency capabilities of Doryteuthis opalescens squid according to prior art, wherein FIG. 1A provides an illustration (left) and photographs (right) of a female squid of this species that switches a white stripe on its mantle from partially transparent (middle) to opaque white (right); FIG. 1B provides a light micrograph of a semi-thin section of the female white stripe on a D. opalescens squid showing tissue strata, where each layer of the cross-section is labeled as follows: epidermis (e), chromatophore layer (c), thick female-specific leucophore layer (1) and underlying muscle (m), and the scale bar is 100 μm; and FIG. 1C illustrates the effects of acetylcholine stimulation on the visibility of the mantle stripe.

FIG. 2 illustrates the cellular and subcellular make-up of the white stripe on the mantle of female Doryteuthis opalescens squid that enables switching of the stripe from nearly transparent to opaque white, according to prior art.

FIGS. 3A through 3C provide electron microscopy images of cephalopod leucophores, according to prior art, wherein FIG. 3A provides TEM images of the leucophore layer from the white stripe of a female Doryteuthis opalescens squid (left) and leucosomes (dark gray) within leucophore cells (right); FIG. 3B provides a SEM image of a fractured Sepia officinalis cuttlefish leucophore showing the cell body containing a disordered arrangement of leucosomes (with an image of an intact cell in the inset) (left), and a TEM image of a cross-section obtained from a Sepia officinalis leucophore showing leucosomes associated with cytoplasmic endoplasmic reticulum strands (right); FIG. 3C provides a TEM image of a cross-section obtained from an Octopus vulgaris leucophore showing the membrane bound leucosomes at the cellular periphery (with the inset showing a magnified view of the same).

FIG. 4 provides a schematic for engineering reflectin-expressing cells with tunable optical properties, in accordance with embodiments of the application.

FIG. 5A illustrates the amino acid sequence for Doryteuthis pealeii reflectin A1 (RfA1) isoform, with the conserved motifs indicated by boxes, while FIG. 5B provides comparison between RfA1 and mammalian proteins in general, in accordance with prior art.

FIG. 6 provides merged fluorescence microscopy images of fixed human cells transfected to express RfA1 that were stained with DAPI and labeled with an antibody pair specific for the protein's histidine-tag, wherein the signals corresponding to DAPI and Alexa 488 fluorophore-conjugated secondary antibody are colored blue and green, respectively (left); and merged fluorescence microscopy images of fixed, transfected cells that were stained with DAPI and labeled with an antibody pair specific for reflectin's unique sequence, wherein the signals corresponding to DAPI and Alexa 488 fluorophore-conjugated secondary antibody are colored blue and green, respectively (right); and wherein the scale bar in both images corresponds to 15 μm, in accordance with embodiments of the application.

FIG. 7 provides fluorescence microscopy images of differently labeled human cells transfected with a vector encoding for the expression of RfA1 in accordance with embodiments of the application, wherein: the top row provides fluorescence microscopy images of fixed RfA1-expressing human cells stained with DAPI and labeled with only the Alexa 488 fluorophore-conjugated secondary antibody; the middle row provides fluorescence microscopy images of fixed RfA1-expressing human cells stained with DAPI and labeled with only a histidine-tag specific primary antibody; and the bottom row provides fluorescence microscopy images of fixed RfA1-expressing human cells stained with DAPI and labeled with only the reflectin sequence specific primary antibody; and wherein the left column shows the channel corresponding to the signals from Alexa 488 fluorophore-conjugated secondary antibody; the middle column shows the channel corresponding to the signals from DAPI (colored blue); and the right column shows the merged signals corresponding to Alexa 488 fluorophore-conjugated secondary antibody and DAPI; and wherein all the scale bars are 20 μm.

FIG. 8 provides fluorescence microscopy images of fixed “mock” transfected human cells, wherein the cells were treated with only the transfection reagents, but no transfection vector of any kind, and then stained with DAPI and labeled with an antibody pair specific for reflectin's unique sequence, in accordance with embodiments of the application, wherein: the left image shows the channel corresponding to the signals from Alexa 488 fluorophore-conjugated secondary antibody; the middle image shows the channel corresponding to the signals from DAPI (colored blue); and the image on the right shows the merged signals corresponding to Alexa 488 fluorophore-conjugated secondary antibody and DAPI; and wherein all the scale bars are 20 μm.

FIG. 9 provides fluorescence microscopy images of fixed untransfected human cells stained with DAPI and labeled with an antibody pair specific for reflectin's unique sequence, in accordance with embodiments of the application, wherein: the left image shows the channel corresponding to the signals from Alexa 488 fluorophore-conjugated secondary antibody; the middle image shows the channel corresponding to the signals from DAPI (colored blue); and the image on the right shows the merged signals corresponding to Alexa 488 fluorophore-conjugated secondary antibody and DAPI; and wherein all the scale bars are 20 μm.

FIG. 10 provides phase contrast images of transfected and untransfected live human cells, in accordance with embodiments of the application, wherein the image on the left shows a phase contrast image of RfA1-expressing human cells, the image on the right shows a phase contrast image of untransfected human cells, and wherein the scale bar in both images is 25 μm.

FIGS. 11A through 11C provide fluorescence microscopy images and plots for the viabilities and cell densities of transfected and untransfected human cells, in accordance with embodiments of the application, wherein: FIG. 11A shows merged fluorescence microscopy images (with the scale bar corresponding to 400 μm) of live, RfA1-expressing (left) and untransfected (right) human cells stained with the live cell-specific calcein AM fluorescent dye (colored green) and the dead cell-specific ethidium homodimer-1 fluorescent dye (colored red);

FIG. 11B illustrates excellent viability of both the transfected and untransfected human cells; and FIG. 11C compares cell densities of transfected and untransfected cells, and wherein the error bars for all plots represent the standard deviation for 3 independent experiments.

FIG. 12A provides fluorescence microscopy images of fixed, RfA1-expressing (left) and untransfected (right) human cells stained with Alexa 555 fluorophore-conjugated wheat germ agglutinin (colored red) wherein the scale bars in both images is 50 μm; while FIG. 12B provides box-and-whisker plots of the average cell areas for transfected and untransfected human cells of FIG. 12A, with the data in the plots corresponding to 3 independent experiments (n=60, *** corresponds to P<0.001), in accordance with embodiments of the application.

FIG. 13 provides TEM images (with the scale bars corresponding to 2 μm), each with close-ups shown as insets (with the scale bars corresponding to 500 nm), of cross-sections obtained from two representative RfA1-expressing human cells (top and bottom), and reveals the presence of electron-dense structures in both cells, wherein the close-up images show a cluster of particles adjacent to the nucleus of the cell (top inset), and a cluster of particles that spans the region between the membrane and the nucleus of the cell (bottom inset), in accordance with embodiments of the application.

FIGS. 14A and 14B provide TEM images of two representative human cells transfected with a vector encoding for the expression of RfA1 in accordance with embodiments of the application (leftmost image for each figure), accompanied by three insets each (to the right) showing close-ups of different cytoplasmic locations for each cell; all images show the presence of electron-dense structures, while the insets corresponding to FIG. 14A additionally show: (1) a close-up image of a cluster of spheroidal particles next to cytoplasmic vesicles and the nucleus; (2) a cluster of spheroidal particles next to a mitochondrion and the nucleus; and (3) an irregular structure next to a large cytoplasmic vesicle; and the insets corresponding to FIG. 14B additionally show: (1) a close-up image of two irregular structures next to the cells' periphery/membrane, (2) an irregular structure next to a cytoplasmic vesicle, and (3) an irregular structure likely in the process of being expelled from the cell; and wherein the scale bar in the FIG. 14A, left, is 2 μm, the scale bar in the FIG. 14B, left, is 1 μm, and the scale bars in all the close-up insets are 500 nm.

FIG. 15A provides a plot of the particle size distribution for electron-dense particles in cells transfected with a vector encoding for the expression of RfA1, in accordance with embodiments of the application, wherein the particle sizes were quantified from 25 independent TEM images and binned to the nearest 0.05 μm; while 15B provides a plot of the particle size distribution for Sepia officinalis leucophores according to prior art for comparison.

FIGS. 16A and 16B provide TEM images of two different untransfected human cells, in accordance with embodiments of the application (leftmost image for each figure), accompanied by three insets each (to the right) showing close-ups of different cytoplasmic locations for each cell; all images show only the presence of various native organelles (e. g., nuclei, mitochondria, and ribosomes) and no foreign structures, while the insets corresponding to FIG. 16A additionally show: (1) a close-up image of a likely vesicle adjacent to a nucleus, (2) various organelles adjacent to a nucleus, and (3) likely mitochondria adjacent to a nucleus; and the insets corresponding to FIG. 16B additionally show: (1) a close-up image of vesicles and organelles adjacent to a nucleus, (2) likely mitochondria between two nuclei, and (3) various organelles adjacent to a nucleus; and wherein the scale bar in the FIG. 16A, left, is 2 μm, the scale bar in the FIG. 16B, left, is 1 μm, and the scale bars in all the close-up insets are 500 nm.

FIG. 17 provides two TEM images of different cross-sections obtained from human cells transfected with a vector encoding for the expression of only red fluorescent protein (RFP), in accordance with embodiments of the application, wherein, the scale bars are 2 μm (for the left image) and 1 μm (for the right image).

FIG. 18 provides an overlay of phase contrast and fluorescence microscopy images of live human cells transfected to express both RfA1 and RFP, in accordance with embodiments of the application, wherein the fluorescence signals corresponding to RFP is colored red and the scale bar is 100 μm.

FIG. 19 provides merged fluorescence microscopy images of fixed RfA1- and RFP-expressing human cells stained with DAPI and labeled with an antibody pair specific for reflectin's unique sequence, wherein the image on the left shows the signals corresponding to DAPI (colored blue) and Alexa 488 fluorophore-conjugated secondary antibody (colored green), and the image on the right shows the signals corresponding to DAPI (colored blue), Alexa 488 fluorophore-conjugated secondary antibody (colored green), and RFP (colored red), and wherein the scale bars in both images are 10 μm, in accordance with embodiments of the application.

FIG. 20 provides fluorescence microscopy images of fixed RfA1- and RFP-expressing human cells stained with DAPI and labeled with only Alexa 488 fluorophore-conjugated secondary antibody (top row), and fluorescence microscopy images of the same cells stained with DAPI and labeled with only the reflectin sequence specific primary antibody (bottom); wherein the leftmost column shows the channel corresponding to the signals from Alexa 488 fluorophore-conjugated secondary antibody, the middle left column shows the channel corresponding to the signals from DAPI (colored blue), the middle right column shows the channel corresponding to the signals from RFP (colored red), and the rightmost column shows the merged fluorescence signals corresponding to Alexa 488 fluorophore-conjugated secondary antibody, DAPI, and RFP; and wherein the scale bars are all 20 μm, in accordance with embodiments of the application.

FIG. 21 provides an immuno-EM image (with the scale bar corresponding to 2 μm) of a cross-section obtained from a representative human cell, which has been labeled with a primary antibody specific for reflectin's unique sequence followed by a complementary secondary antibody conjugated to a gold nanoparticle (left), and an inset with a close-up image (with the scale bar corresponding to 500 nm) of a small cluster of RfA1 structures (large gray spheres) (right), which are specifically labeled by antibody-conjugated gold nanoparticles (small black dots), in accordance with embodiments of the application.

FIG. 22 provides a representative TEM image of a cross-section obtained from RfA1- and RFP-expressing human cells, which shows the presence of electron-dense spheroidal particles (left), and an inset with a close-up image of a cluster of spheroidal particles next to cytoplasmic vesicles and the nucleus (right), wherein the scale bars are 2 μm for the left image and 500 nm for the right image, in accordance with embodiments of the application.

FIG. 23 provides a schematic (top portion) and a representative reflection-mode quantitative phase microscopy image (bottom) of transfected human cells on a reflective substrate before RfA1 expression has begun (left) and after RfA1 expression has begun (right), wherein the scale bars for both images are 15 μm, in accordance with embodiments of the application.

FIG. 24 provides reflection-mode quantitative phase microscopy images (left column), fluorescence microscopy images (middle column), and refractive index maps (right column) obtained for recently-transfected human cells, which do not yet express RfA1 and RFP (top row) and for transfected human cells expressing RfA1 and RFP (bottom row), wherein the red outline in the bottom row of images indicates cells that expressed RfA1 and RFP, and wherein the scale bars for all images are 15 μm, in accordance with embodiments of the application.

FIG. 25 provides plots of the average refractive index as a function of time after transfection of human cells with a vector encoding for both RfA1 and RFP, wherein the bottom plot shows the average refractive index of non-fluorescent cells that do not express RfA1 and RFP and the top plot shows the average refractive index of fluorescent cells that express both proteins, and wherein the error bars represent the standard deviation for 3 independent experiments, in accordance with embodiments of the application.

FIG. 26 provides transmission-mode quantitative phase microscopy images (left column) and corresponding optical pathlength maps (right column) obtained for the human cells expressing RfA1 which were imaged during a 30-minute time frame, wherein the top row corresponds to the start of the time frame and the bottom row—to the end of the time frame, wherein the white arrows in the left-side images label RfA1-based structures with higher phases, and the white arrows in the right-side images label dark red spots with longer optical pathlengths relative to the immediate surroundings, and wherein the scale bars are 10 μm, in accordance with embodiments of the application.

FIG. 27 provides a plot of the calculated refractive index as a function of the diameter for RfA1-based structures (top set of circles) and for analogous cytoplasmic regions (bottom set of squares) specifically found near the peripheries of human cells that express RfA1, in accordance with embodiments of the application.

FIG. 28 provides a plot of the measured refractive index as a function of the leucosome diameter for Sepia officinalis cuttlefish leucophores (left), and a histogram of the number of leucosomes as a function of the refractive index for Sepia officinalis cuttlefish leucophores, according to prior art.

FIG. 29 provides schematics (left) and representative brightfield microscopy images (right) of a sandwich-type configuration, wherein the middle layer consists of an RfA1-expressing human cell culture, after exposure to media with a 117 mM (top) or a 217 mM (bottom) NaCl concentration, in accordance with embodiments of the application.

FIGS. 30A and 30B provide representative histograms of the count (number of pixels) as a function of the intensity (grayscale values) for RfA1-expressing human cells in a sandwich-type configuration after exposure to media with a 117 mM (FIG. 30A) or a 217 mM (FIG. 30B) NaCl concentration, along with the corresponding histograms obtained for the cell-free background in the same sandwich-type configuration (black bars in FIGS. 30A and 30B), in accordance with embodiments of the application.

FIG. 31 provides schematics (left) and the corresponding brightfield microscopy images (right) of a sandwich-type configuration, wherein the middle layer consists of an untransfected human cell culture, after exposure to media with a 117 mM (top) or a 217 mM (bottom) NaCl concentration, wherein the scale bars are 200 μm for both images, in accordance with embodiments of the application.

FIG. 32 provides representative histograms of the count (number of pixels) as a function of the intensity (grayscale values) for untransfected human cells in a sandwich-type configuration after exposure to media with a 117 mM (left) or a 217 mM (right) NaCl concentration, along with the corresponding histograms obtained for the cell-free background in the same sandwich-type configuration (black bars in both plots), in accordance with embodiments of the application.

FIG. 33A provides merged fluorescence microscopy images (with the scale bars corresponding to 400 μm) of live, RfA1-expressing human cells stained with the live cell-specific calcein AM fluorescent dye and the dead cell-specific ethidium homodimer-1 fluorescent dye, after exposure to media with a 117 mM (left) or a 217 mM (middle left) NaCl concentration, showing the signals corresponding to the calcein AM (colored green) and ethidium homodimer-1 dyes (colored red); a plot of the fraction of live cells as a function of the NaCl concentration for the RfA1-expressing human cells (middle right); and a plot of the cell density as a function of the NaCl concentration for the RfA1-expressing human cells (right), wherein the error bars represent the standard deviation for 3 independent experiments; while FIG. 33B provides the same data and analysis in the same layout for untransfected cells, in accordance with embodiments of the application.

FIG. 34A provides fluorescence microscopy images (with the scale bars corresponding to 50 μm) of fixed, RfA1-expressing human cells stained with Alexa 555 fluorophore-conjugated wheat germ agglutinin after exposure to media with a 117 mM (left) or a 217 mM (middle) NaCl concentration, showing the corresponding fluorescence signals (colored red); and box-and-whisker plots of the average cell areas as a function of the NaCl concentration for RfA1-expressing human cells, wherein the data in the plots corresponds to 3 independent experiments (n=60, P<0.05 is considered to be statistically significant); while FIG. 34B provides the same data and analysis in the same layout for untransfected cells, in accordance with embodiments of the application.

FIG. 35 provides the representative total transmittance (left) and total reflectance (right) spectra obtained for a sandwich-type configuration comprising RfA1-expressing human cells after exposure to media with a 117 mM or a 217 mM NaCl concentration, in accordance with embodiments of the application.

FIG. 36 provides the total transmittance (left) and total reflectance (right) spectra obtained for a sandwich-type configuration comprising untransfected human cells after exposure to media with a 117 mM or a 217 mM NaCl concentration, along with the corresponding total transmittance and reflectance spectra obtained for a sandwich-type configuration prepared without any cells at all provided for comparison, in accordance with embodiments of the application.

FIG. 37 provides representative diffuse transmittance (left) and diffuse reflectance (right) spectra obtained for a sandwich-type configuration comprising RfA1-expressing human cells after exposure to media with a 117 mM or a 217 mM NaCl concentration, in accordance with embodiments of the application.

FIG. 38 provides plots of the average diffuse transmittances (left) and average diffuse reflectance (right) for a sandwich-type configuration comprising either RfA1-expressing or untransfected human cells after exposure to media with a 117 mM (left columns) or a 217 mM (right columns) NaCl concentration, wherein the error bars represent the standard deviation for at least 5 independent experiments (n=5, ** corresponds to P<0.005, where P<0.05 is considered to be statistically significant), in accordance with embodiments of the application.

FIG. 39 provides representative diffuse transmittance (left) and diffuse reflectance (right) spectra obtained for a sandwich-type configuration comprising untransfected human cells after exposure to media with a 117 mM or a 217 mM NaCl concentration, along with the corresponding spectra obtained for sandwich-type configurations having no cells at all provided for comparison, in accordance with embodiments of the application.

FIG. 40 provides schematics (left) and representative digital camera images (right) of a cuvette containing an aqueous RfA1 solution with a 117 mM (top) or a 217 mM (bottom) NaCl concentration, in accordance with embodiments of the application.

FIG. 41 provides representative total transmittance (left) and total reflectance (right) spectra obtained for aqueous RfA1 solutions with a 117 mM or a 217 mM NaCl concentration, in accordance with embodiments of the application.

FIG. 42 provides representative diffuse transmittance (left) and diffuse reflectance (right) spectra obtained for aqueous RfA1 solutions with a 117 mM or a 217 mM NaCl concentration, in accordance with embodiments of the application.

FIG. 43 provides a plot of the volume fraction as a function of the particle diameter for aqueous RfA1 solutions with a 117 mM or a 217 mM NaCl concentration (left), and a plot of the average particle diameter for aqueous RfA1 solutions as a function of the NaCl concentration, wherein the error bars represent the standard deviation for 6 independent experiments (right), in accordance with embodiments of the application.

FIG. 44 provides plots of the average diffuse transmittances (left) and average diffuse reflectances (right) for aqueous RfA1 solutions as a function of the NaCl concentration (circles), along with the corresponding data for aqueous solutions without any RfA1 (squares) provided for comparison, wherein the error bars represent the standard deviation for 5 independent experiments, in accordance with embodiments of the application.

### DETAILED DISCLOSURE

Turning now to the schemes, images, and data, various embodiments are directed toward molecular tools to controllably regulate optical properties of living biological systems, including whole cells, subcellular organelles and biomolecules, and/or whole organs, tissues, or parts thereof, comprising such cells. In many embodiments, the tunable optical properties comprise refractive index. In a number of embodiments, cephalopod protein reflectin, or another biomolecule derived from or similar to reflectin, is expressed within a biological cell, wherein the plasmid encoding for expression of the reflectin biomolecule, the expression level of such reflectin biomolecules, and the structure and/or aggregation state of the reflectin biomolecule are used to regulate the reflectin biomolecule's refractive index and the refractive index of its immediate environment within the engineered cell.

In several embodiments, reflectin protein, or any number of its analogs or derivatives, including unnatural reflectin biomolecules, is expressed within a biological cell from a nucleic acid vector (e.g., plasmid, viral vector, RNA, etc.). In many embodiments, reflectin constructs can be introduced into a living biological cell such that its expression is regulated. In many embodiments, the living biological cell is a mammalian cell. In some embodiments, the living biological cell is a human cell. In many embodiments, a particular isoform of reflectin is transfected into a host cell, which may optimize the optical properties of the host cell and facilitate control over thereof. In some embodiments, reflectin A1 (RfA1) isoform is transfected into mammalian cells.

In many embodiments, conformation and aggregation of expressed reflectin within a host cell is controlled via external stimuli. In some such embodiments, external stimuli include, but are not limited to: NaCl, ionic strength of the host cell's environment, aromatic compounds, acetylcholine, any other chemical, biological, biophysical, electrical, and mechanical stimulus known to affect conformation and aggregation of reflectin and/or any of reflectin's isoforms, and any combination thereof.

In many embodiments, reflectin biomolecules expressed within living biological cells, according to the methods of the instant application, form multimeric photonic structures. In many such embodiments, the multimeric photonic structures comprising reflectin biomolecules allow one to control the refractive index and/or transparency of the host cell, or particular constituents within the host cell (e.g., organelles). In a similar manner, embodiments are also directed to adjusting the refractive index and/or transparency of biological systems and/or tissues within a multicellular organism (e.g., mouse) and/or adjusting the refractive index and/or transparency of the entire multicellular organism. In various embodiments, engineering of cells that encompass reconfigurable biomolecular photonic architectures and, thus, possess tunable light-transmitting and light-reflecting capabilities is inspired by the subcellular structures and adaptive optical functionalities of cephalopod leucophores, and especially dynamic leucophores found in the mantle of female Doryteuthis pealeii and Doryteuthis opalescens.

In many embodiments, desirable optical properties, such as refractive index and extent of light scattering, are engineered into living cells via the incorporation of reflectin-based or reflectin-like structures. In some such embodiments, mammalian or other cells and organoids are engineered to possess cephalopod-inspired optical functionalities, such as, for example, stimuli-responsive dynamic iridescence or mechanically-reconfigurable coloration (Williams, T. L. et al. Dynamic pigmentary and structural coloration within cephalopod chromatophore organs. Nat. Commun. 10, (2019), the disclosure of which is incorporated herein by reference).

In many embodiments, intracellular structures comprising reflectin biomolecules are used as biomolecular reporters for quantitative phase imaging in 2D or 3D of varied cellular processes across typically non-transparent biological specimens. For example, in some embodiments, subcellular reflectin structures resulting from the expression of reflectins by a transfected cell, may help visualize expulsion of extracellular vesicles in such cells. Reflectins' unique amino acid sequences, which are orthogonal to mammalian systems, and their validated high refractive indices make them ideal candidates for biomarker applications in mammalian (including human) cells.

In many embodiments, reflectins' diverse stimuli-responsive self-assembly properties and ease of expression according to the methods of the instant application, together, enable real-time adaptive refractive index matching of specific cells to their surroundings and, thus, facilitate imaging of entire living tissues with improved clarity and resolution on conventional optical microscopes. Therefore, clearing techniques for study of living tissues, organs, and/or whole organisms that utilize the methods of the instant application can be developed.

In some embodiments, transfected biological cells expressing reflectin, or another reflectin-based or reflectin-derived biomolecule, according to the methods of the instant application are used for further in vivo studies of structure-property relationships of reflectins, including many reflectin isoforms, as well as the molecular and cellular biology of mollusks in general.

It will be understood that the embodiments of the invention described herein are not intended to be exhaustive or to limit the invention to precise forms disclosed. Rather, the embodiments selected for description have been chosen to enable one skilled in the art to practice the invention.

Proteins known as reflectins are important components of cephalopod skin, wherein they enable some of the optical functionalities of cephalopod skin cells, such as, for example, the functionalities of cephalopod leucophore and iridophore cells (Chatterjee, A. et al. An introduction to color-changing systems from the cephalopod protein reflectin. Bioinsp. Biomim. 13; Crookes, W. J. et al. Reflectins: the unusual proteins of squid reflective tissues. Science 303, 235-238 (2004); Levenson, R. et al. Molecular mechanism of reflectin's tunable biophotonic control: opportunities and limitations for new optoelectronics. APL Mater. 5, the disclosures of which is incorporated herein by reference). With a few exceptions, reflectins' generalized amino acid sequences include variable linker regions that are separated by conserved motifs with the highly general form encompassing the three identified motif types, i.e.: MEPM(X)₂M(X)MDF(X)₅DS(X)₁₀ (SEQ. ID. No. 1), PER(X)₂DM(X)₄MD(X)₂G(X)₁₁P (SEQ. ID. No. 2), and (X)D(X)₅MD(X)₅M(X)₆ (SEQ. ID. No. 3). These sequences are unusual because they have a low percentage of common aliphatic amino acids (e.g. alanine, leucine, isoleucine) and a high percentage of aromatic amino acids (e.g. tyrosine and tryptophan), while also being enriched in arginine, asparagine, and methionine. This peculiar composition is thought to be directly responsible for reflectins' remarkably diverse self-assembly properties and their unusually high refractive indices (Zhao, H. et al. On the distribution of protein refractive index increments. Biophys. J. 100, 2309-2317 (2011); Kramer, R. M. et al. The self-organizing properties of squid reflectin protein. Nat. Mater. 6, 533-538 (2007), the disclosures of which is incorporated herein by reference). Specifically, reflectins not only form the spheroidal leucosomes found in leucophores (Mathger L. M., et al. Bright white scattering from protein spheres in color changing, flexible cuttlefish skin. Adv. Funct. Mater. 23, 3980-3989 (2013); Hanlon, R. T., et al. White reflection from cuttlefish skin leucophores. Bioinsp. Biomim. 13, the disclosures of which is incorporated herein by reference), and the membrane-enclosed platelets found in iridophores (DeMartini, D. G. et al. Structures, organization, and function of reflectin proteins in dynamically tunable reflective cells. J. Biol. Chem. 291, 4058-4068 (2016); DeMartini, D. G. et al. Membrane invaginations facilitate reversible water flux driving tunable iridescence in a dynamic biophotonic system. Proc. Natl. Acad. Sci. 110, 2552-2556 (2013); Ghoshal, A. et al. Experimental determination of refractive index of condensed reflectin in squid iridocytes. J. R. Soc. Interface 11, (2014), the disclosures of which is incorporated herein by reference) in vivo, but they also readily assemble into nanoparticles (Tao, A. R. et al. The role of protein assembly in dynamically tunable bio-optical tissues. Biomater. 31, 793-801 (2010); Levenson, R. et al. Cyclable condensation and hierarchical assembly of metastable reflectin proteins, the drivers of tunable biophotonics. J. Biol. Chem. 291, 4058-4068 (2016); Naughton. K. L. et al. Self-Assembly of the Cephalopod Protein Reflectin. Adv. Mater. 28, 8405-8412 (2016), the disclosures of which is incorporated herein by reference), microfibers, hexagonal plates (Guan Z. et al. Origin of the reflectin gene and hierarchical assembly of its protein. Curr. Biol. 27, 2833-2842 (2017), the disclosure of which is incorporated herein by reference), and thin films (Phan, L. et al. Reconfigurable infrared camouflage coatings from a cephalopod protein. Adv. Mater. 25, 5621-5625 (2013); Phan, L. et al. Infrared invisibility stickers inspired by cephalopods. J. Mater. Chem. C 3, 6493-6498 (2015); Ordinario, D. D. et al. Protochromic Devices from a Cephalopod Structural Protein. Adv. Optical Mater. 5, (2017), the disclosures of which is incorporated herein by reference) in vitro. For some of these nano- and micro-structures, the application of different chemical stimuli can even modulate their aggregation state (e.g. NaCl and ionic strength for the nanoparticles) or lead to disassembly/reassembly (e.g. aromatic compounds for the hexagonal plates). Furthermore, reflectin-based structures have been proven to possess high refractive indices in varied contexts, with average values of ˜1.44 reported for condensed platelets in squid iridophores, ˜1.51 observed for leucosomes in cuttlefish leucophores, and ˜1.54 to ˜1.59 measured for reflectin films on solid substrates. Overall, reflectins possess a host of unique, attractive, and controllable optical characteristics.

### Molecular Tools for Biological Expression of Reflectin Biomolecules

A number of embodiments are directed towards molecular tools to transgenically express reflectin protein, or related to reflectin natural or unnatural biomolecules, within a living biological cell. In many embodiments, reflectin or its variant is introduced within the cell via an exogenous nucleic acid vector. Any appropriate nucleic acid vector may be utilized, including (but not limited to) DNA constructs, RNA constructs, DNA plasmid, and viral vector. Any appropriate means to provide a cell with a nucleic acid may be utilized, including (but not limited to) chemical transfection, electroporation, lipid transfection, viral vector transduction, and gene gun. In some embodiments, a nucleic construct capable of expressing reflectin protein, including any of its isoforms, or another reflectin-derived/reflectin-mimicking natural or unnatural biomolecule, is integrated within the host genome, which can create cells stably expressing the reflectin biomolecule of interest. Any appropriate mechanism for genome integration can be utilized, including (but not limited to) CRISPR/Cas system, non-homologous end joining, viral vectors, or any appropriate mechanism.

In several embodiments, reflectin biomolecule can be expressed in various different living biological cells, including prokaryotic cells and eukaryotic cells, such as animal or plant cells, or any cell type capable of transgenic expression. In many embodiments, reflectin biomolecule is transgenically expressed in cell types that are utilized in a laboratory or clinical setting. In some embodiments, reflectin biomolecule is transgenically expressed in human cell lines, human organoids, non-human primate, mice, rat, guinea pig, dog, chicken, zebrafish, Drosophila melanogaster, yeast, Arabidopsis spp. (e.g., A. thaliana), or any other appropriate model organism.

Any appropriate natural or unnatural reflectin-based sequence can be utilized for expression in accordance with multiple embodiments. In a number of embodiments, reflectin biomolecule sequence is derived from Doryteuthis pealeii and Doryteuthis opalescens, which express a number of isoforms of reflectin, including reflectin A1, reflectin A2, reflectin B1, reflectin C1, and other isoforms or homologues. In various embodiments, a partial sequence of natural reflectin protein is expressed, such as, for example, a sequence resulting in a biomolecule that is a truncated reflectin protein. In some embodiments, various sequences are added to natural reflectin protein sequence, which may be useful for various laboratory or clinical applications. For instance, a Histidine tag may be added to reflectin sequence to assist in detection and/or purification via antibodies that recognize the Histidine tag. Within this application, reflectin biomolecule is to be understood to be any natural or unnatural biomolecule that is either a natural isoform of reflectin or a reflectin variant derived from or mimicking reflectin protein, including any of its isoforms.

In some embodiments, a reflectin biomolecule coding sequence is a codon optimized for expression in particular cells, such as eukaryotic cells. Eukaryotic cells may be those of or derived from a particular organism, such as a mammal, or other model organism, including (but not limited to) human, non-human primate, mice, rat, guinea pig, dog, chicken, zebrafish, D. melanogaster, yeast, Arabidopsis, or any other appropriate species. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently, or most frequently, used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA). Accordingly, genes can be tailored for optimal gene expression in a given organism based on codon optimization. Codon usage tables are readily available, for example, at the “Codon Usage Database”, and these tables can be adapted in a number of ways (see Y. Nakamura, et al. Codon usage tabulated from the international DNA sequence databases: status for the year 2000 Nucl. Acids Res. 28, 292 (2000), the disclosure of which is incorporated herein by reference). In some embodiments, one or more codons (e.g., 1, 2, 3, 4, 5, 10, 15, 20, 25, 50, or more, or all codons) in a sequence encoding a reflectin protein corresponds to the most frequently used codon for a particular amino acid.

Provided within the sequence listing are exemplary sequences of reflectin biomolecules. For a full list of sequences and their explanation, see Table 1. SEQ. ID No. 5 is a naturally occurring protein sequence from D. pealeii. SEQ. ID No. 4 is an example of a Reflectin A1 DNA gene sequence with an N-terminal histidine tag from D. pealeii. Many other isoforms of reflectin sequences are provided. Although specific biomolecule sequences of Reflectin A1 are provided, various embodiments are directed to biomolecule sequences of Reflectin A1 with variations. Accordingly, various embodiments are directed to biomolecule sequences of Reflectin A1 with 99%, 98%, 97%, 95%, 90%, 85%, 80%, 75%, or even lower homology. As noted above, many reflectin biomolecules possess a conserved motif in one or more domains (e.g., N-terminal, internal, and/or C-terminal). Such motifs are also described in Table 1 and illustrated as SEQ ID Nos: 1-3. As such, many embodiments are directed to reflectin biomolecules possessing 1, 2, or 3 sequence motifs selected from SEQ ID Nos: 1-3.

Several embodiments relate to vector systems comprising one or more vectors, typically designed for expression of reflectin biomolecule transcripts in prokaryotic or eukaryotic cells. For example, reflectin transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990), the disclosure of which is incorporated herein by reference. Alternatively, a recombinant expression vector can be transcribed and translated extracellularly, for example using T7 promoter regulatory sequences and T7 polymerase. Vectors may be introduced and propagated in a prokaryote (e.g., in form of plasmid). In some embodiments, a prokaryote is used to amplify copies of a vector (or an intermediate cloning vector), with some vectors amplified for introduction into a eukaryotic cell. Vectors may also include helper vectors that support the introduction of a reflectin biomolecule into a cell (e.g., a plasmid as part of a viral vector packaging system). In some embodiments, a prokaryote is used to express one or more vectors to produce expressed products, such as (for example) to provide a source of peptide products, which in turn can be utilized for delivery to a biological cell or organism. Expression of proteins in prokaryotes is most often carried out in Escherichia coli with vectors containing constitutive or inducible promoters directing the expression of either fusion or non-fusion proteins. In some embodiments, fusion vectors add a number of amino acids to a reflectin peptide product encoded therein, such as to the amino terminus of the recombinant protein, which may assist in peptide production. Such fusion vectors may serve one or more purposes, including (but not limited to): (i) increase expression of recombinant protein; (ii) increase the solubility of the recombinant protein; and (iii) aid in the purification of the recombinant protein by acting as a ligand in affinity purification. In some scenarios, a fusion expression vector includes a proteolytic cleavage site is introduced at the junction of the fusion moiety and the recombinant protein to enable separation of the recombinant protein from the fusion moiety subsequent to purification of the fusion protein. Example fusion expression vectors include pGEX (Pharmacia Biotech Inc; Smith and Johnson, 1988. Gene 67: 31-40, the disclosure of which is incorporated herein by reference), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein. Examples of suitable inducible non-fusion E. coli expression vectors include pTrc (Amrann et al. (1988) Gene 69:301-315, the disclosure of which is incorporated herein by reference), and pET lid (Studier et al. GENE EXPRESSION TECHNOLOGY: METHODS IN ENZYMOLOGY, Academic Press, San Diego, Calif. (1990) 60-89, the disclosure of which is incorporated herein by reference).

In some embodiments, a vector is capable of driving expression of one or more sequences in mammalian cells using a mammalian expression vector. Examples of mammalian expression vectors include pCDM8 (B. Seed, An LFA-3 cDNA encodes a phospholipid-linked membrane protein homologous to its receptor CD2 Nature 329, 840-842 (1987) and pMT2PC (R. J. Kaufman, P. Murtha, and M. V. Davies, EMBO J. Translational efficiency of polycistronic mRNAs and their utilization to express heterologous genes in mammalian cells. 6, 187-195 (1987)), the disclosures of which are each incorporated herein by reference. When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al. MOLECULAR CLONING: A LABORATORY MANUAL. 2nd ed., Cold Spring Harbor Laboratory, Cold Spring Harbor Laboratory Press, Cold Spring Harbor, N.Y., 1989, the disclosure of which is incorporated herein by reference.

In some embodiments, a recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al. 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBOJ. 8: 729-733) and immunoglobulins (Baneiji, et al. 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al. 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (Campes and Tilghman, 1989. Genes Dev. 3: 537-546), the disclosures of which are each incorporated herein by reference.

In some embodiments, a regulatory element is operably linked to one or more elements of a reflectin biomolecule nucleic acid sequence so as to drive the expression of the reflectin biomolecule. In various embodiments, an expression vector includes a promoter upstream of the reflectin biomolecule nucleic acid sequence. In some embodiments, a poly-A signal is provided downstream of the reflectin biomolecule nucleic acid sequence.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a reflectin biomolecule sequence, and optionally downstream of a regulatory element operably linked to the reflectin biomolecule sequence. In some embodiments, reflectin biomolecule expression vectors may be provided, and optionally delivered to a cell.

In some embodiments, reflectin biomolecule is a part of a fusion protein comprising one or more heterologous protein domains. A reflectin fusion peptide may include only a portion of the reflectin protein (e.g, specific reflectin domains), and/or any additional protein sequence(s), and/or a linker sequence between any domains. Examples of additional protein domains that may be fused to a reflectin peptide include, without limitation, epitope tags and reporter gene sequences. Non-limiting examples of epitope tags include Histidine (His) tags, V5 tags, FLAG tags, influenza hemagglutinin (HA) tags, Myc tags, VSV-Gtags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, mCayenne, cyan fluorescent protein (CFP), yellow fluorescent protein (YFP), and autofluorescent proteins including blue fluorescent protein (BFP). A reflectin peptide (or portion of refectin peptide) may be fused to other gene sequences that encode a protein, such that the reflectin peptide provides a direct quantitative phase report of the encoded protein.

In some embodiments, a reporter gene, which includes (but is not limited to) glutathione-S-transferase (GST), horseradish peroxidase (RP), chloramphenicol acetyltransferase (CAT) beta-galactosidase, beta-glucuronidase, luciferase, green fluorescent protein (GFP), HcRed, DsRed, mCayenne, cyan fluorescent protein (CFP), yellow fluorescent protein (YFP), and autofluorescent proteins including blue fluorescent protein (BFP), is introduced into a cell to encode a gene product which serves as a marker by which to measure the alteration or modification of expression of the reflectin or reflectin biomolecule gene product. In various embodiments, DNA molecules encoding a reflectin peptide and/or expression markers may be introduced into the cell via a vector.

In some embodiments, methods are directed towards delivering one or more polynucleotides, such as or one or more vectors as described herein, one or more transcripts thereof, and/or one or proteins transcribed therefrom, to a host cell. Some embodiments are directed towards cells produced by such methods, and organisms (such as animals, plants, or fungi) comprising or produced from such cells. In some embodiments, a reflectin biomolecule expression vector is delivered to a cell.

Conventional viral and non-viral based gene transfer methods can be used to introduce nucleic acids in mammalian cells or target tissues. Such methods can be used to administer nucleic acids encoding reflectin biomolecule to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon. 40 TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al. in Current Topics in 45 Microbiology and Immunology Doerfler and Bohm (eds) (1995); and Yu et al. Gene Therapy 1:13-26 (1994), the disclosures of which are incorporated herein by reference.

Methods of non-viral delivery of nucleic acids include lipofection, nucleofection, microinjection, biolistics, virosomes, liposomes, immunoliposomes, polycation or lipid nucleic acid conjugates, naked DNA, artificial virions, and agent-enhanced uptake of DNA. Lipofection reagents are sold commercially (e.g., Transfectam and Lipofectin).

The use of RNA or DNA viral based systems for the delivery of nucleic acids take advantage of highly evolved processes for targeting a virus to specific cells in the body and trafficking the viral payload to the nucleus. Viral vectors can be administered directly to organisms in vivo or they can be used to treat cells in vitro. Conventional viral based systems could include retroviral, lentiviral, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al. J. Viral. 66:2731-2739 (1992); Johann et al. J. Viral. 66:1635-1640 (1992); Sommnerfelt et al. Viral. 176:58-59 (1990); Wilson et al. J. Viral. 63:2374-2378 (1989); Miller et al. J. Viral. 65:2220-2224 (1991); PCT/US94/05700), the disclosures of which are each incorporated herein by reference.

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (AAV) vectors may also be used to transduce cells with target nucleic acids, e.g., in the in vitro production of nucleic acids and peptides, and for in vivo and ex vivo gene therapy procedures (see, e.g., West et al. Virology 160:38-47 (1987); U.S. Pat. No. 4,797,368; WO 93/24641; Katin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al. Mol. Cell. Biol. 5:3251-3260 (1985); Tratschin, et al. Mol. Cell. Biol. 4:2072-2081 (1984); Hermonat & Muzyczka, PNAS 81:6466-6470 (1984); and Samulski et al. J. Viral. 63:03822-3828 (1989), the disclosures of which are each herein incorporated by reference.

Packaging cells are typically used to form virus particles that are capable of infecting a host cell. Such cells include HEK 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, the disclosure of which is incorporated herein by reference.

In some embodiments, a host cell is transiently or non-transiently transfected with one or more vectors expressing reflectin biomolecule. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to ARPE-19, HeLa, HEK 293, HEK 293T, 3T3, PC-3, RPTE, A375, A549, SW480, SW620, jurkat, Bcl-1, BC-3, MEFs, Hep G2, COS, COS-1, COS-6, CHO, K562 cells, and transgenic varieties thereof. Cell lines are available from a variety of sources known to those with skill in the art (see, e.g., the American Type Culture Collection (ATCC) (Manassas, Va.)).

In some embodiments, a cell transfected with one or more vectors described herein is used to establish a new cell line comprising one or more vector-derived sequences such that reflectin biomolecule is expressed. In some embodiments, a cell transiently transfected with reflectin biomolecule expression vector as described herein (such as by transient transfection of one or more vectors, or transfection with RNA), is used to establish a new cell line comprising cells that have altered refractive index. In some embodiments, cells transiently or non-transiently transfected with one or more vectors described herein, or cell lines derived from such cells, are used in various applications in which alteration of cellular, subcellular, or biomolecular refractive index is desired (e.g., microscopy).

In some embodiments, one or more vectors described herein are used to produce a non-human transgenic animal or transgenic plant. In some embodiments, the transgenic animal is a mammal, such as a mouse, rat, or rabbit. In certain embodiments, the organism or subject is a plant. In certain embodiments, the organism or subject or plant is algae. Methods for producing transgenic plants and animals are known in the art, and generally begin with a method of cell transfection, such as described herein. Transgenic animals are also provided, as are transgenic plants, especially crops and algae.

Various embodiments are directed towards kits containing any one or more of the vectors disclosed in the above methods and compositions. In some embodiments, a kit comprises a vector system and instructions for using the kit. In some embodiments, a vector system comprises a regulatory element operably linked to a reflectin biomolecule sequence. Components of a kit may be provided individually or in combinations, and may be provided in any suitable container, such as a vial, a bottle, or a tube.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium phosphate buffer, sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 5 to about 10.

### Applications of Biological Expression of Reflectin Biomolecules

Reflectins possess high refractive index that can be modulated via controllable changes to these proteins' conformation and aggregation state. Accordingly, in some embodiments, controllable expression and/or controllable conformation/aggregation of reflectin biomolecules is utilized to modulate refractive index of host cell biomolecules, subcellular organelles, whole cells, tissues, organs, or even whole organisms. In many such embodiments modulated refractive index can be utilized in various applications, including various microscopy techniques, or other visualization applications.

In some embodiments, transgenic transfection of reflectin biomolecule into biological cells is utilized to model cephalopod cells, which have been difficult to culture in vitro. It has now been demonstrated that expressing reflectin in a mammalian cell model (e.g., HEK 293) allows these transfected cells to form leucosome-like structures that have refractive indices similar to those reported for cuttlefish leucophores. As HEK 293 cells are easy to culture and the transformation of these cells to produce reflectin biomolecules is straightforward, this provides a model system with which to study leucophores and other cephalopod cells that contain reflectin.

In a number of embodiments, reflectin biomolecule structures and aggregates internalized by cells are utilized for various microscopy techniques. In many such embodiments, distinct and tunable optical properties (including high refractive index) of reflectin biomolecule structures/aggregates allow their visualization with a light microscope. In some embodiments, particular biomolecules within a cell, subcellular organelles, whole cells and/or tissues containing reflectin biomolecule structures/aggregates can be visualized when the reflectin biomolecule structures/aggregates attached to, associated with, or contained within are used to adjust the refractive index of the host. In many embodiments, by controlling location and time of reflectin biomolecule expression within a cell, the cell's refractive index can be locally adjusted and temporally controlled. Accordingly, in some embodiments, biological cells can be transfected with an exogenous nucleic acid vector to express reflectin biomolecules, and the expression of reflectin biomolecule can be monitored by visualizing and/or measuring the cell's refractive index. In some embodiments, particular proteins and/or organelles are tagged with a reflectin biomolecule such that their refractive index is altered, allowing localized and temporal visualization and/or measurement. Within this application, an exogenous biomolecule is to be understood to be a biomolecule that is introduced into the biological cell. For instance, exogenous DNA can be introduced into a biological cell by transfection or viral transduction.

Several embodiments are direct to bio-photonic architectures constructed from a single or multiple distinct reflectin biomolecules within a cell. Photonic architectures are a variety of architectures that cause light to be reflected, refracted, diffracted, or scattered in distinct, definable ways. In a number of embodiments, cells transfected to produce reflectin biomolecule show clustering of spherical reflectin biomolecule-containing particles that can be externally stimulated to aggregate or fold into various conformations/structures and, as such, alter the way these particles scatter light (thereby, appearing darker or lighter). In many embodiments, the choice of an external stimulus and/or a reflectin isoform/variant being used affects the photonic reflectin biomolecule-containing architectures within the cell. In some embodiments, the engineering of cells with tunable optical properties according to the methods of the instant application enables living biological optical waveguides, which control how light is transported over long distances, and “internet of things” (IoT) type devices that can be implanted within and run from within the body.

Engineering of Living Cells with Tunable Optical Properties

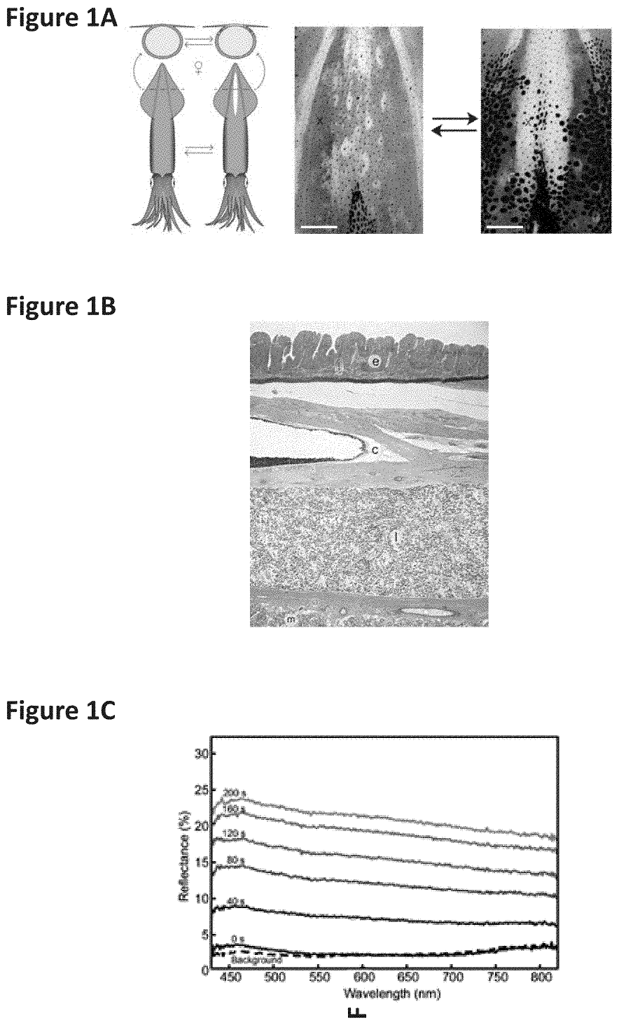

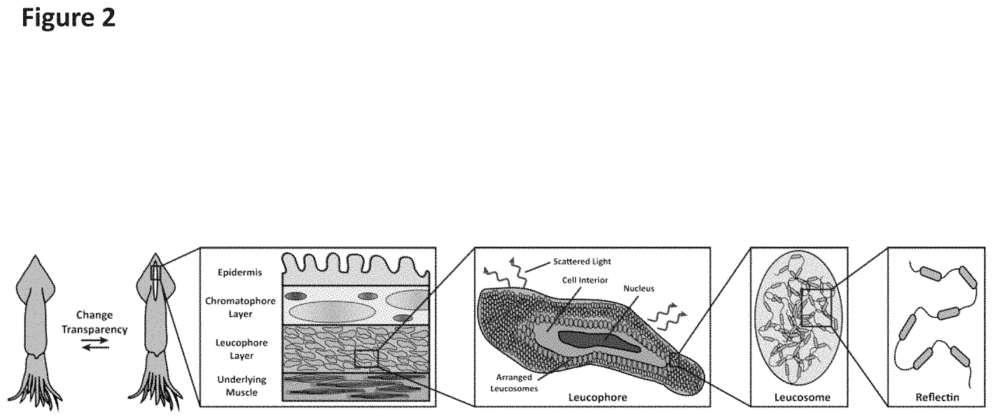

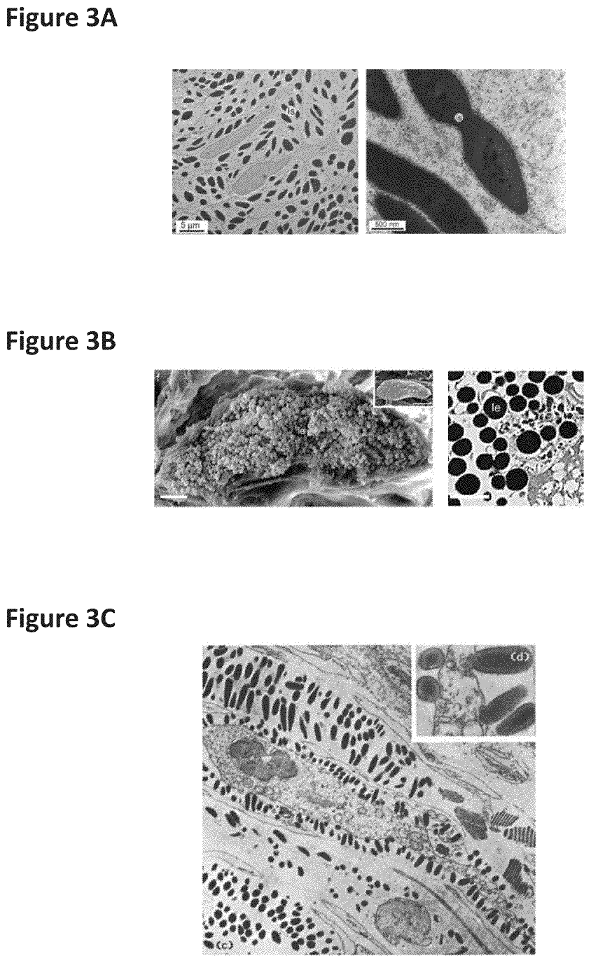

In nature, adaptive transparency can be observed, to some extent, in many cephalopods (i.e., octopuses, squids, and cuttlefish) which are capable of camouflage, including literal “vanishing acts” (Phan, L. et al. Dynamic materials inspired by cephalopods. Chem. Mater. 28, 6804-6816 (2016); Mäthger, L. M. et al. Mechanisms and behavioural functions of structural coloration in cephalopods. J. R. Soc. Interface 6, S149-S163 (2009); Hanlon, R. T. et al. Cephalopod Behaviour (Cambridge University Press, New York, 2018); Cloney, R. A. et al. Chromatophore organs, reflector cells, iridocytes and leucophores in cephalopods. Amer. Zool. 23, 581-592 (1983); DeMartini, D. G. et al. Dynamic biophotonics: female squid exhibit sexually dimorphic tunable leucophores and iridocytes. J. Exp. Biol. 216, 3733-3741 (2013), the disclosures of which is incorporated herein by reference). Indeed, these animals can dynamically alter how their skin transmits, absorbs, and reflects light due to the functionality of unique optical components, which include pigment containing chromatophore organs, typically narrowband-reflecting iridophore cells, and broadband-reflecting leucophore cells. For example, the female Doryteuthis opalescens squid avoids unwanted aggression by switching a stripe on its mantle from nearly transparent (weakly scattering) to opaque white (strongly scattering) (FIGS. 1A-1C). This feat represents a fascinating case study of adaptive biological optics and is thought to be achieved by means of a specialized layer that contains tunable leucophores (FIGS. 1B and 2). In general, leucophores in octopus and cuttlefish skin have been shown to contain disordered arrangements of proteinaceous structures called leucosomes, which range in diameter from hundreds of nanometers to several microns and can be membrane-bound or localized throughout the cells' bodies (FIGS. 2 and 3A-3C) (Froesch, D. et al. On leucophores and the chromatic unit of Octopus vulgaris. J. Zool. 186, 163-173 (1978), the disclosure of which is incorporated herein by reference). Typically, the disordered leucosome arrangements are static, allowing cuttlefish leucophores to scatter (i.e., diffusely reflect) incident visible light via a Mie-type mechanism and, thus, behave as passive broadband reflectors that produce bright white coloration. However, the leucosome arrangements within the leucophores in the mantle of female D. opalescens squid are dynamic, rather than passive, and as such, their broadband reflectances can be reversibly modulated by, for example, injection of acetylcholine into the surrounding tissue, as demonstrated in FIGS. 1A-1C. Notably, the exact mechanisms underpinning such functionality are not yet completely understood. However, many of the internalized photonic architectures that enable the optical functionalities of cephalopod skin cells, including leucophores, are known to be composed of reflectins—proteins with a host of unique and controllable optical characteristics. Accordingly, in many embodiments, dynamic cephalopod leucophores and their constituent light-scattering photonic architectures, further comprised of reflectin proteins, serve as an inspiration for the design and engineering of cellular systems with tunable optical properties, as well as for the construction of bio-optical systems not found in the natural world.

A number of embodiments are directed towards cells transfected to produce reflectin biomolecule and, therefore, comprising stimuli-responsive photonic reflectin biomolecule-containing architectures, methods of manufacture thereof, and use thereof. In turn, in many embodiments, the reflectin biomolecule architectures internalized by the transfected cells, endow the cells with tunable optical properties, i.e., the ability to change appearance by modulating the transmission of light. In many embodiments the transfected cells with tunable optical properties are mammalian cells. In many embodiments, the transfected cells contain designer, stimuli-responsive, photonic architectures enabling the cells of the application to change transparency and appearance on demand. In many embodiments, the subcellular photonic architectures of the transfected cells comprise reflectin biomolecule and function similarly to squid's leucosomes contained within tunable leucophores. In some embodiments, reflectin biomolecule is reflectin protein. In many embodiments, each transfected cell comprises a cell transfected with a plasmid encoding for overexpression of reflectin biomolecule. In some embodiments, each transfected cell comprises a cell transfected with a plasmid encoding for expression of reflectin biomolecule and another one or more proteins/biomolecules foreign to the cell.

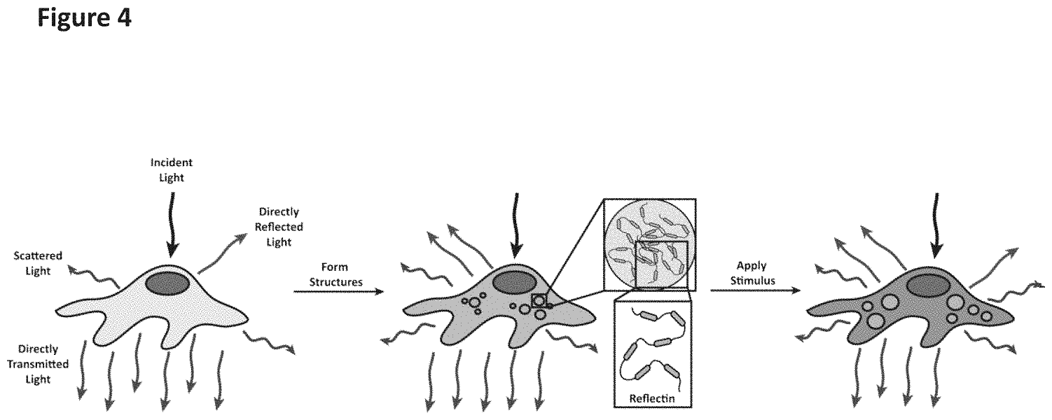

FIG. 4 illustrates many embodiments, wherein introduction of reflectin biomolecule into a biological cell endows the living cell with adjustable optical properties. More specifically, a biological cell lacking reflectin biomolecule of embodiments directly transmits most of the incident visible light with relatively minimal scattering, which makes it appear mostly transparent (FIG. 4, left). In contrast, upon introduction of reflectin biomolecule into the cell in accordance with many embodiments, reflectin biomolecule within the cell forms leucosome-like cytoplasmic aggregates (i.e., photonic architectures) randomly distributed throughout the cell's interior with uniquely high local refractive indices, which, in turn, causes the scattering of some of the incident visible light, making the host cell less transparent compared to a cell lacking reflectin biomolecules (FIG. 4, middle). In many such embodiments, reflectin biomolecule-based photonic architectures within a cell have refractive indices that differ significantly from the surrounding cytoplasm and, consequently, diffusely transmit and/or diffusely reflect (i.e., scatter) some of the incident visible light, thereby making the host cells less transparent (i.e., more opaque), in analogy to passive cuttlefish leucophores (FIG. 4, middle).

In some embodiments, in absence of any external stimuli that can affect reflectin biomolecule's conformation and aggregation, the reflectin biomolecule-based photonic aggregates are randomly distributed throughout the transfected cell's interior as disordered arrangements and provide a degree of cell opaqueness. However, in various embodiments, upon exposure of the transfected cell of the instant application to a stimulus known to influence reflectin assembly, such as, for example, NaCl, the geometries and/or arrangements of the photonic reflectin biomolecule architectures within the cell are reconfigured, and begin to diffusely transmit and/or diffusely reflect (i.e. scatter) a different amount of the incident visible light (FIG. 4, right). Accordingly, in many such embodiments, application of an external stimulus known to affect reflectin's conformation and or aggregation alters the transfected cell's transparency/opaqueness, in analogy to tunable squid leucophores (FIG. 4, right). In many embodiments, the reversible application of one or more chemical stimuli affecting reflectin biomolecule assembly leads to reversible changes in the opaqueness of the transfected cells of the application, providing the cells with adaptive transparency.

In many embodiments, the cell platform for integration of the photonic architectures comprising reflectin biomolecules and engineering of cells with adaptive transparency are chosen for their ability to reliably express various recombinant proteins and to accumulate highly-overexpressed foreign proteins within cytoplasmic inclusion bodies or phase-separated aggregates. In certain embodiments, the cells are engineered, such that one or more cellular components are characterized by a reflectin biomolecule. For example, reflectin biomolecules may accumulate with, on, or within a cellular component, including (but not limited to) an organelle, protein, membrane, cytoskeleton, ribosome, or any other cellular component of interest. In many such embodiments, the cells of choice are mammalian cells. In some embodiments the mammalian cells are human cells. In some embodiments the mammalian cells chosen according to the instant application are human embryonic kidney (HEK) 293 cells, which possess all the necessary characteristics (Thomas, P. et al. HEK293 cell line: a vehicle for the expression of recombinant proteins. J. Pharmacol. Toxicol. Methods. 51, 187-200 (2005); Rajan, R. S. et al. Specificity in intracellular protein aggregation and inclusion body formation. Proc. Natl. Acad. Sci. U.S.A. 98, 13060-13065 (2001); Schuster, B. S. et al. Controllable protein phase separation and modular recruitment to form responsive membraneless organelles. Nat. Commun. 9, https://doi.org/10.1038/s41467-018-05403-1 (2018), the disclosures of which is incorporated herein by reference).

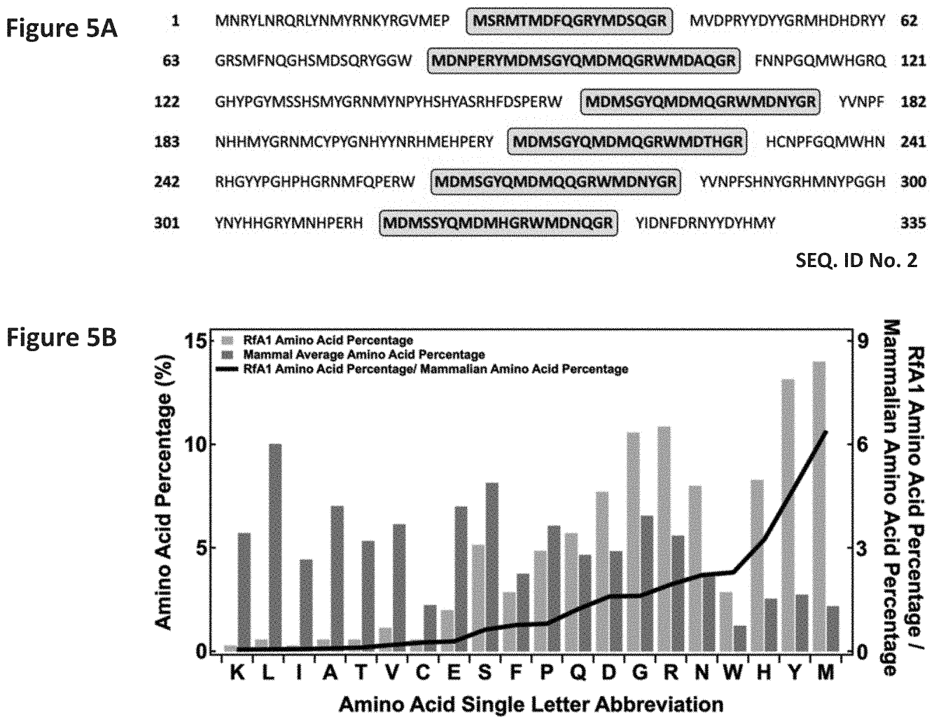

In many embodiments, wherein reflectin biomolecule is the building block for the subcellular photonic architectures of the application, the choice of the specific reflectin biomolecule to be used in the cell transfection is optimized for the biomolecule's ability to form optically active structures with high refractive indices, as well as for the amino acid composition to be maximally distinct from that of a host cell's (e.g., orthogonal to mammalian cells). In many embodiments, reflectin biomolecule of choice for the transfection of mammalian cells is reflectin isoform A1 (RfA1). Specifically, RfA1 isoform is known to: 1) feature refractive indices that are among the largest known for any protein (Zhao, H. et al. On the distribution of protein refractive index increments. Biophys. J. 100, 2309-2317 (2011), the disclosure of which is incorporated herein by reference); 2) assemble into a diverse array of ionic strength-responsive structures both in vitro and in vivo in squid skin cells; and 3) possess amino acid sequences that differ dramatically from those of mammalian proteins (FIGS. 5A and 5B).

### Formation of Reflectin Biomolecule-Based Photonic Architectures in Transfected Cells

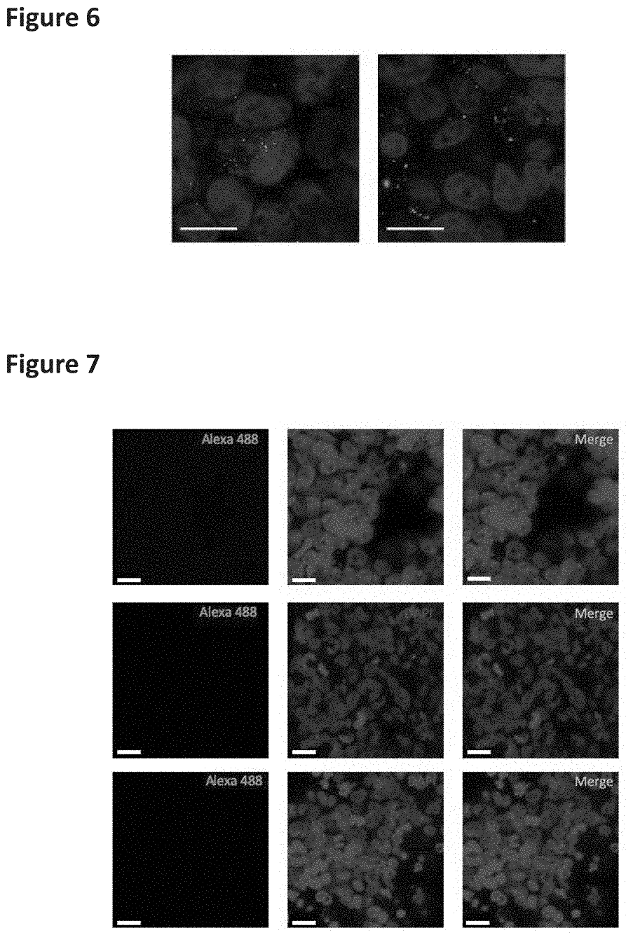

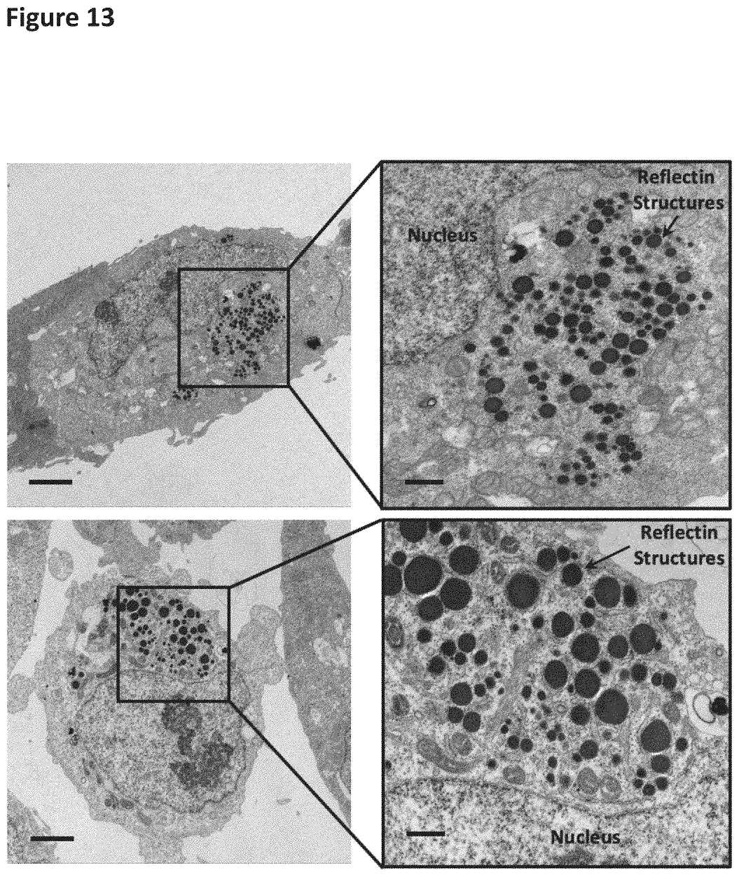

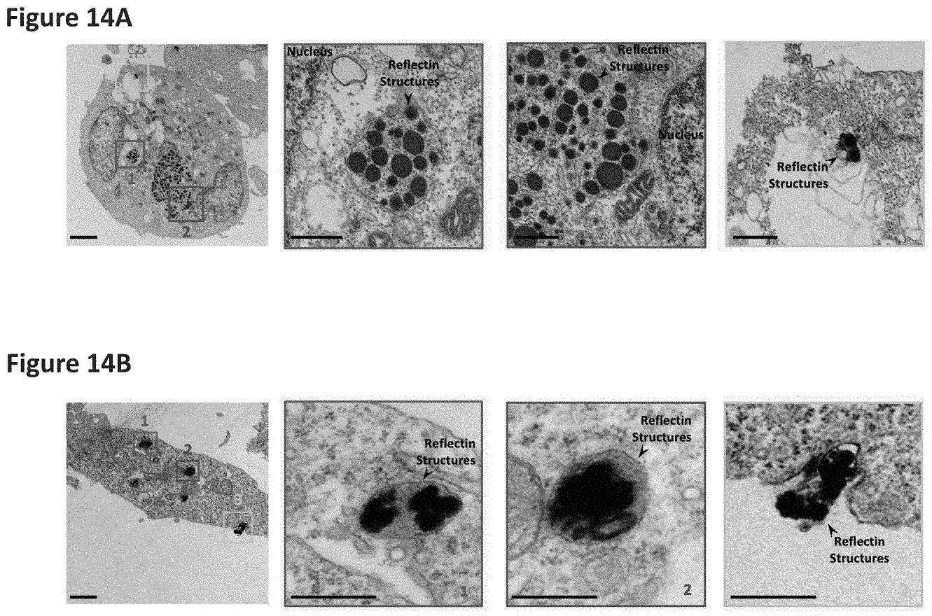

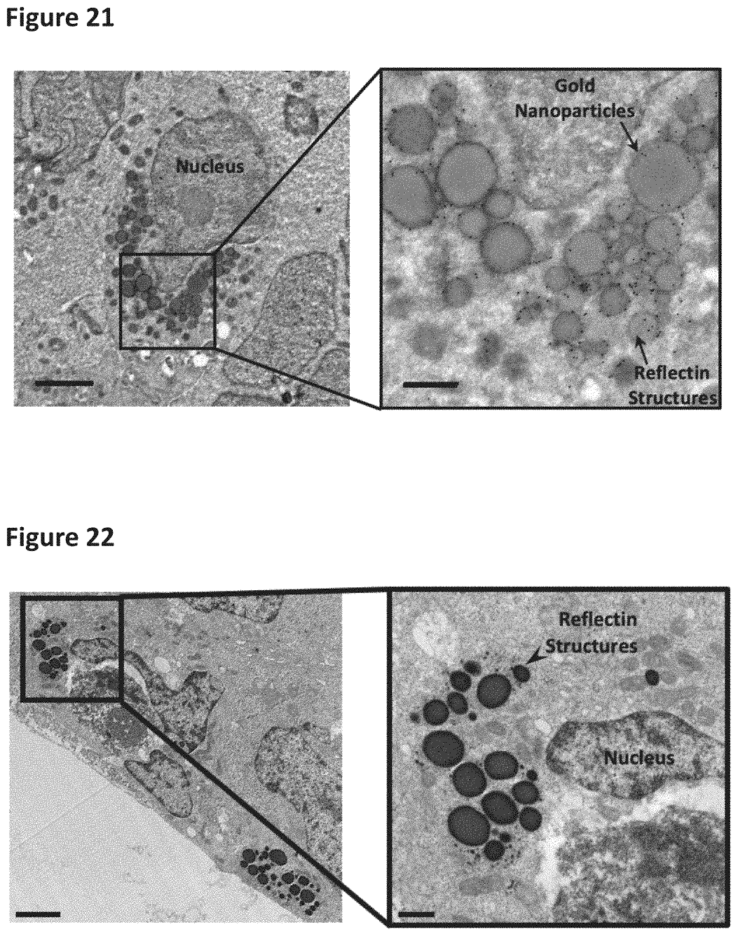

FIGS. 6-22 illustrate formation and characterization of the cells transfected to produce reflectin biomolecule and of the reflectin biomolecule-based photonic architectures contained within, according to many embodiments. In many such embodiments, living biological cells (especially mammalian cells) are engineered according to methods described herein to produce large quantities of reflectin biomolecule. To this end, in many embodiments, the cells of choice, such as, for example, mammalian HEK 293 cells, produce a Doryteuthis pealeii RfA1, or another desired isoform, variant or reflectin, or a biomolecule based on reflectin. In several embodiments, RfA1 or other reflectin biomolecules are introduced via expression vectors as described herein (e.g., transfection or transduction). As various examples, FIGS. 6-22, discussed below in detail, provide visualization and evaluation of HEK 293 cells transfected with RfA1 according to the methods of the instant application, wherein the transfected cells are assessed and compared to untransfected cells via various microscopy methods applied to appropriately labeled live and fixed cells. More specifically, immunofluorescence microscopy images in FIG. 6, along with various controls provided in FIGS. 7-9, illustrate successful expression of reflectin by mammalian (i.e., HEK 293) cells; data in FIGS. 10-12 further illustrates viability of transfected cells and underscores their morphological similarity (in both live and fixed states) to corresponding untransfected cells; FIGS. 13-20 describe reflectin structures that form within the transfected cells and show their distinct localization within the transfected cell; FIGS. 21-22, together with FIGS. 13, 14A, and 14B, confirm reflectin composition of the foreign clusters observed within the transfected cells.

### Viability and Health of Transfected Cells

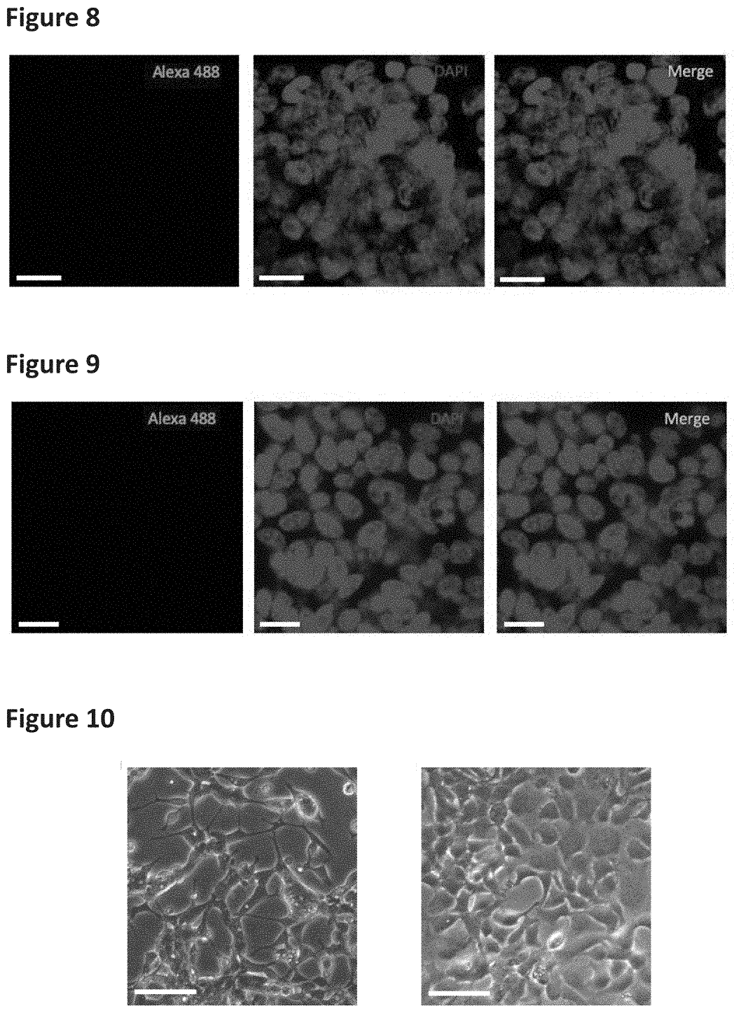

FIG. 6 illustrates successful expression of reflectin by mammalian cells of the instant application, wherein fixed and appropriately stained and labeled transfected cells are analyzed by immunofluorescence microscopy. In this figure, the overlaid fluorescence microscopy images of fixed RfA1-transfected cells stained with the nuclear marker 4′,6′-diamidino-2-phenylindole (DAPI) and immunolabeled with an antibody pair specific for reflectin's N-terminal histidine tag reveals that the nuclei (colored blue) of the transfected cells are surrounded by small reflectin aggregates (colored green), suggesting the successful expression of reflectin protein by the cells (FIG. 6, left). Similarly, the overlaid fluorescence microscopy images of fixed RfA1-transfected cells stained with DAPI, but immunolabeled with an antibody pair specific for reflectins' unique sequence, again, reveal that the nuclei (colored blue) are surrounded by reflectin aggregates (colored green), corroborating the successful expression of reflectin by the cells (FIG. 6, right). In contrast, the control fluorescence microscopy images of RfA1-transfected cells for which immunolabeling was attempted with any member of the antibody pairs omitted did not reveal any fluorescence signals (FIG. 7). Furthermore, the fluorescence microscopy images of both, the “mock” transfected cells (i.e., ones treated with the transfection reagents but not the RfA1 vector), and the untransfected cells, for both of which immunolabeling was attempted with the reflectin-specific antibody pair, also did not reveal any fluorescence signals (FIGS. 8 and 9). Accordingly, in many embodiments, the transfected cells of the instant application successfully express the non-native reflectin biomolecule and localize it within punctate aggregates.

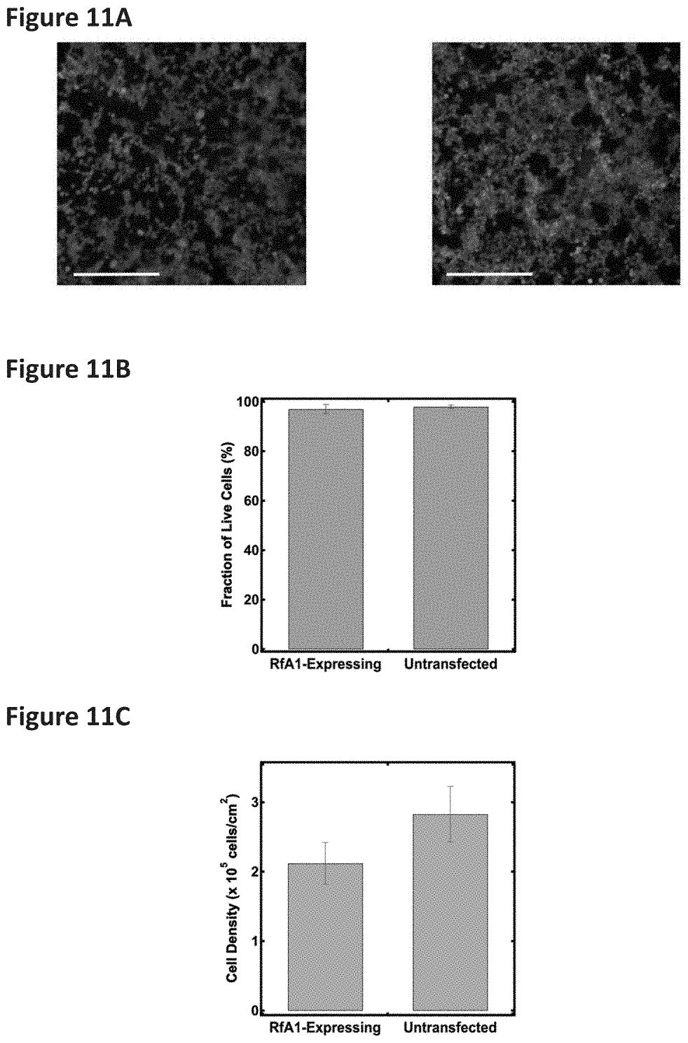

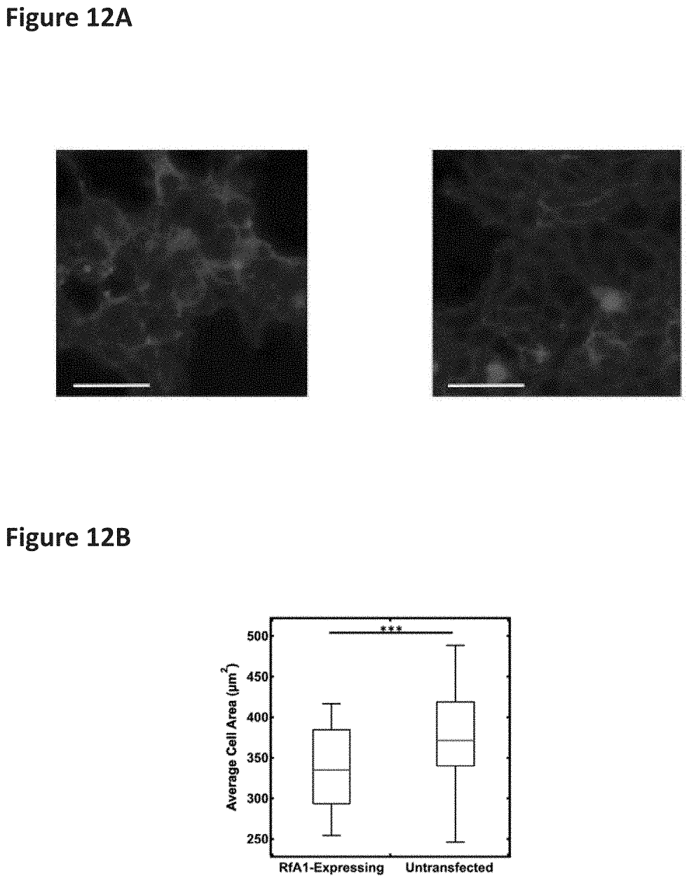

FIGS. 10, 11A-C, 12A, and 12B further illustrate viability of the cells transfected according to the methods of the instant application and demonstrate their morphological similarities to the corresponding untransfected cells. More specifically, these figures provide data and analysis for exemplary mammalian cells transfected with a vector encoding for the expression of histidine-tagged RfA1, wherein live and fixed, transfected and untransfected cells are stained with different markers and visualized with fluorescence microscopy. To this end, FIG. 10 compares the morphology of exemplary transfected (left) and untransfected (right) cells via phase contrast microscopy of unstained live cells and demonstrates that the transfected cells feature slightly rounded morphologies (FIG. 10, left), as compared to spread-out morphologies of untransfected cells (FIG. 10, right). Furthermore, FIGS. 11A-11C compare viability and density of exemplary transfected and untransfected cells via fluorescence microscopy (FIG. 11A) and quantitative analysis (FIGS. 11B and 11C) of cells stained with both the live cell-specific calcein AM fluorescent dye and the dead cell-specific ethidium homodimer-1 fluorescent dye. According to this analysis, the viability and density of the cells transfected according to many embodiments (FIGS. 11A, left) is almost indistinguishable from the corresponding parameters for the untransfected cells (FIGS. 11A, right), with transfected cells showing a viability of 97 (±2) % (FIG. 11B) and a density of 2.1 (±0.2)×10⁵ cells/cm² (FIG. 11C), versus the viability of 98 (±1) % (FIG. 11B) and the slightly higher density of 2.8 (±0.4)×10⁵ cells/cm² (FIG. 11C) demonstrated by the corresponding untransfected cells. Moreover, FIGS. 12A and 12B further compare exemplary transfected and untransfected cells via fluorescence microscopy of fixed cells stained with fluorophore-tagged wheat germ agglutinin. This imagining technique and subsequent analysis reveal that the area of fixed HEK 293 cells transfected with RfA1 is 338 (±50) μm² (FIG. 12A, left, and FIG. 12B), which is a slightly smaller value than that of 375 (±50) μm² measured for the untransfected cells used as control (FIG. 12A, right, and FIG. 12B). Accordingly, in many embodiments, the expression of the non-native reflectin biomolecule alters the morphologies and areas of the transfected cells at least somewhat, but does not significantly impact their overall health in the short term.

### Characterization of Subcellular Reflectin-Based Structures of Transfected Cells

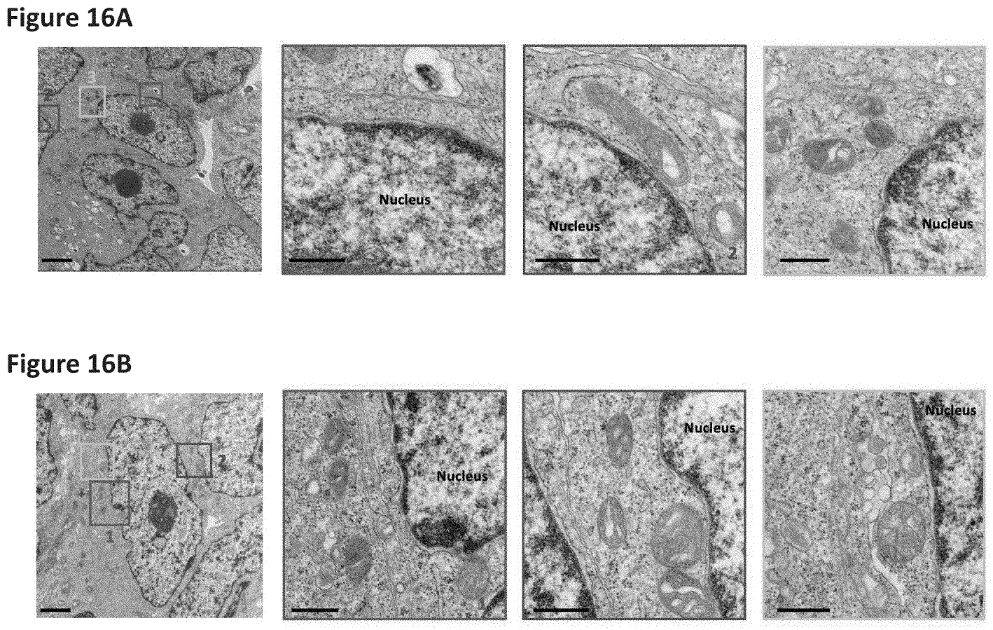

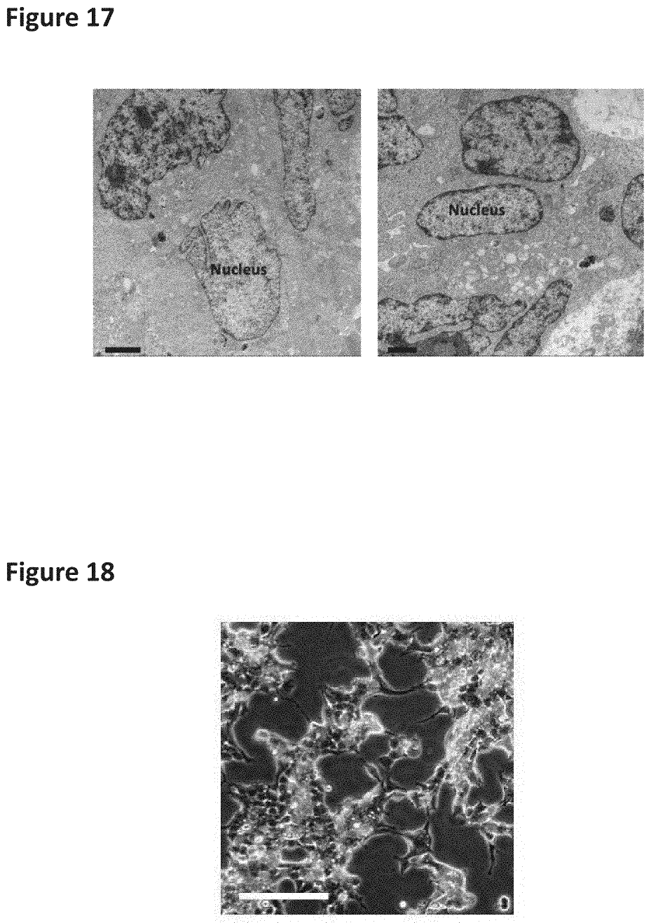

FIGS. 13-17 illustrate and characterize the reflectin-based structures and aggregates formed within the cells engineered to produce reflectin according to many embodiments of the application. More specifically, FIGS. 13, 14A, and 14B provide transmission electron microscopy (TEM) images of thin cross-sections prepared from fixed, resin-embedded RfA1-expressing BEK 293 cells of many embodiments. These TEM images reveal the presence of distinct arrangements of intracellular structures with large sizes and high electron densities found alongside the cell's organelles (e.g., the nucleus, mitochondria, and ribosomes). Furthermore, these distinct arrangements, believed to comprise reflectin, constitute >20% of the cellular cross-sections' areas. Moreover, the observed subcellular reflectin arrangements appear to represent two types of structures: 1) spheroidal particles with diameters of approximately ˜50 nm to ˜250 nm primarily located in clusters within the cells' cytoplasm (FIGS. 13, 14A, and 15A); and 2) irregularly-shaped structures with diameters greater than ˜250 nm often located closer to (or outside) the cells' membranes and peripheries (FIGS. 13, 14B, and 15A). In contrast, the TEM images of cross-sections prepared from untransfected cells (FIGS. 16A and 16B), or from cells transfected to produce red fluorescent protein (RFP), which is known to be aggregation-prone (Müller-Taubenberger, A. et al. Recent advances using green and red fluorescent protein variants. Appl. Microbiol. Biotechnol. 77, (2007), the disclosure of which is incorporated herein by reference), instead of reflectin (FIG. 17), did not show any high electron density structures resembling the ones found for the reflectin-expressing cells of the instant application. Not to be bound by any theory, the TEM analysis indicates that the engineered cells of the application sequester subcellular reflectin within spheroidal particles, coalesce such particles into irregularly-shaped structures, and expel the larger structures into the surrounding environment. Together, this analysis of TEM images affords insight into the sizes, aggregation states, and intracellular distributions of the reflectin biomolecule-based structures within the transfected cells of many embodiments.

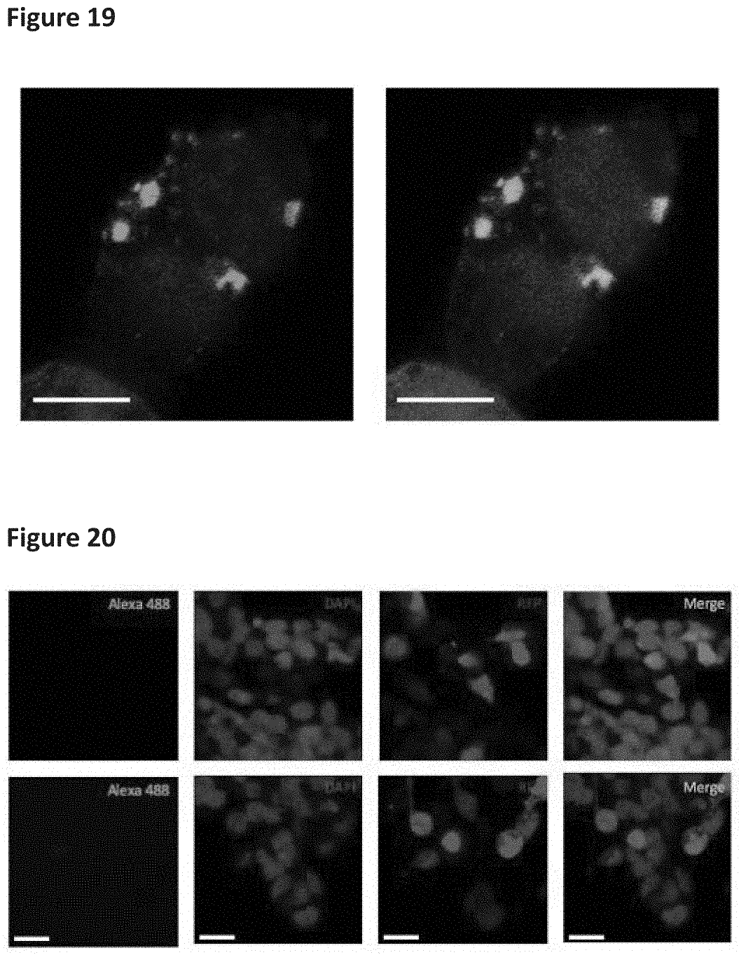

FIGS. 18-20 provide further illustrative examples of how reflectin producing cells of the instant application handle and distribute the reflectin protein. More specifically, FIG. 18 provides an overlay of phase contrast and fluorescence microscopy images of BEK 293 cells transfected with a vector encoding for the expression of both histidine-tagged RfA1 and a distinct biomolecular reporter RFP. This image of live RfA1- and RFP-transfected cells indicates that nearly two thirds of the transfected cells express RfA1, as gauged from the fraction of the cell population that exhibits red fluorescence associated with the RFP reporter. Furthermore, the merged fluorescence microscopy images obtained for fixed RfA1- and RFP-expressing cells stained with DAPI and immunolabeled with an antibody pair specific for reflectins' sequence provided in FIG. 19 (left) reveal that the nuclei (colored blue) are in close proximity to reflectin aggregates (colored green), in agreement with the comparable images for the reflectin-expressing cells provided in FIG. 6. FIG. 19 also reveals that the localized fluorescence from the immunolabeled reflectin-based structures (colored green) does not precisely overlap with the more dispersed fluorescence from the independent RFP reporter proteins (colored red in FIG. 19, right), suggesting that the two biomolecules are distributed throughout the cells differently, and further supporting the idea of unique handling of reflectin by cells. Furthermore, the control fluorescence microscopy images of fixed RfA1- and RFP-transfected cells, for which labeling was attempted with either the primary or secondary member of the reflectin-specific antibody pair omitted, only show fluorescence signals associated with RFP, but not with specific immunolabeling (FIG. 20). Accordingly, in many embodiments, the transfected cells of the instant application readily express reflectin biomolecule in high yield and distribute it as variable-sized aggregates throughout the cells' interiors in a particular fashion.

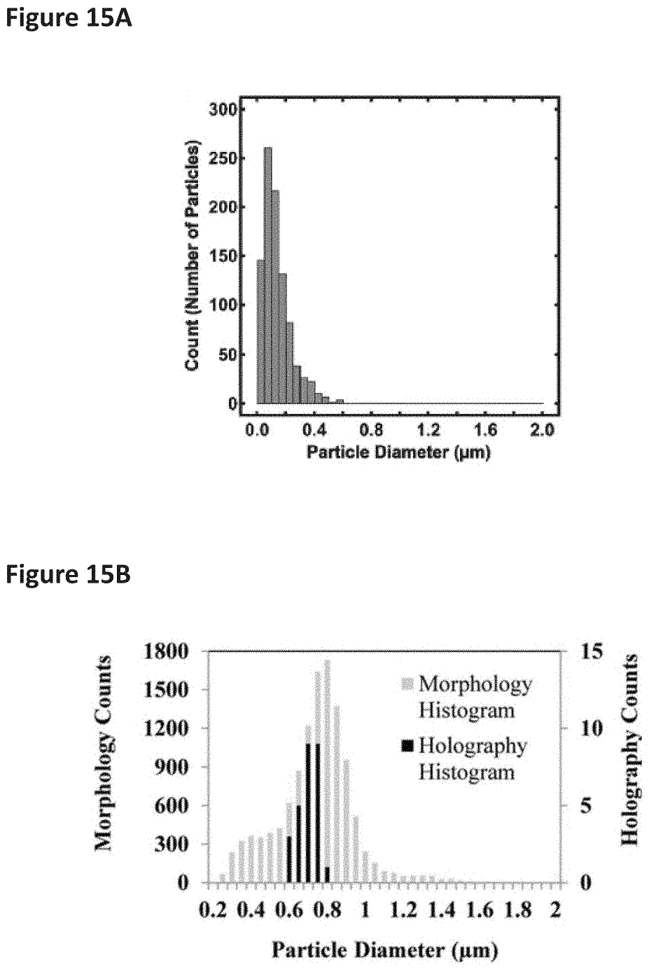

FIGS. 21 and 22 further illustrate that the aggregates observed within the transfected cells of the instant application do, indeed, comprise reflectin. More specifically, FIG. 21 provides a representative immuno-electron microscopy (immuno-EM) image of ultra-thin cross-sections prepared from fixed, cryoprotectant-treated RfA1- and RFP-expressing cells, wherein the sections were immunolabeled via the treatment with a primary antibody specific for reflectins' sequence, followed by the treatment with a complementary secondary antibody conjugated to a gold nanoparticle. Accordingly, the immuno-EM image in FIG. 21 shows the presence of clusters of electron-dense structures (dark gray spheres) distributed throughout the cells' interiors alongside the usual organelles, including, for example, the nucleus. Furthermore, these electron-dense clusters appear to constitute a significant fraction of the cellular cross-sections' areas (and, presumably, of the cellular volumes), and to consist of mostly spheroidal structures with diameters of tens to hundreds of nanometers. In general, the aggregates observed in FIG. 21 appear to be similar in most respects, i.e. size, shape, cytoplasmic location and distribution, to those observed in the TEM image of cells that express RfA1 (FIGS. 13, 14A and 14B), as well as to those observed in the TEM images of cells that expressed both RfA1 and RFP (FIG. 22). Most importantly, higher-magnification immuno-EM images of the cellular sections (for example, FIG. 21, inset) show that the dark spheroidal aggregates/structures are selectively labeled by the antibody-conjugated gold nanoparticles (small black dots), thus, demonstrating that the structures comprise RfA1. Notably, the subcellular RfA1-based structures observed in all of the high-resolution immuno-EM and TEM images of the transfected cells of the instant application (FIGS. 13, 14A and 14B, 21 and 22) closely resemble the reflectin-based leucosomes found in the electron microscopy images of octopus, cuttlefish, and squid leucophores, with the exception that the size distribution found for the subcellular reflectin structures of the instant application is somewhat different from that of, for example, cuttlefish leucosomes (FIGS. 3B and 15B). Together, the images and data provided in FIGS. 6-22 illustrate the appearance and some of the properties characteristic of the cells transfected to express reflectin biomolecule and of the reflectin biomolecule-based structures observed within, according to many embodiments of the instant application.

### Optical Properties of Transfected Cells Comprising Reflectin-Based Photonic Architectures

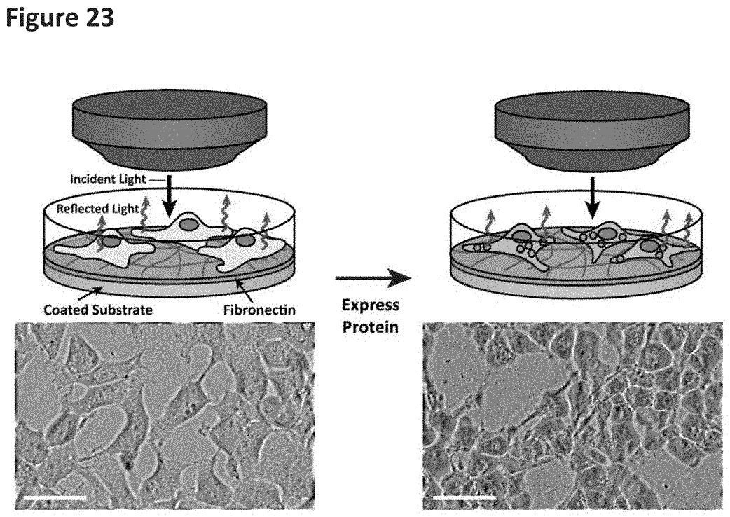

FIGS. 23-28 illustrate how the cells transfected to produce reflectin according to many embodiments of the instant application interact with light. More specifically, these figures, discussed below in detail, provide many illustrative examples, wherein HEK 293 cells transfected with a vector encoding for the expression of RfA1 according to the methods of the instant application are assessed via Reflection-Mode Low Coherence Quantitative Phase Microscopy (RLC-QPM), in tandem RLC-QPM and fluorescence microscopy, and Transmission-Mode Low Coherence Quantitative Phase Microscopy (TLC-QPM) to demonstrate that, among other features: 1) the reflectin-based subcellular structures affect the way the transfected host cells reflect light; 2) the reflectin producing cells have consistently higher refractive indices than the corresponding untransfected cells; 3) the subcellular reflectin structures are not static, but move around the cytoplasm of the transfected host cells, and affect local refractive indices of the transfected host cells; 4) the refractive indices of the subcellular reflectin-based structures depend on the size and aggregation state of these structures, allowing for manipulation of global optical properties of the host cells.

Towards this end, FIG. 23 provides visualization of transfected cell cultures of embodiments with Reflection-Mode Low Coherence Quantitative Phase Microscopy (RLC-QPM). RLC-QPM technique measures how the incident light changes its phase when reflected from various objects (such as living cells positioned on a substrate, as seen in the top portion of FIG. 23), and, as such, enables the generation of phase images, which quantitatively represent the observed phase shifts (bottom portion of FIG. 23) (Yamauchi, T. et al. Low-coherent quantitative phase microscope for nanometer-scale measurement of living cells morphology. Optics express 16, 12227-12238 (2008); Yamauchi, T. et al. Transportable and vibration-free full-field low-coherent quantitative phase microscope. SPIE 10503, (2018); Park, Y. K., Depeursinge, C., & Popescu, G. Quantitative phase imaging in biomedicine. Nat. Photonics 12, 578-589 (2018), the disclosures of which is incorporated herein by reference). First, as seen on the left-hand side of FIG. 23, the RLC-QPM images obtained for recently-transfected cells, which did not have enough time to express RfA1, show that the cells have expected spread-out morphologies of untransfected cells, comparable to the cell morphologies observed in the standard phase contrast microscopy images of untransfected cells (FIG. 10, right). Furthermore, the observed phase difference between the cells' bodies (relatively dark gray areas) and the glass substrate (relatively light gray areas) is moderate, although the bodies feature some regions with a greater phase difference (small gray-black spots) that likely corresponds to organelles (FIG. 23, left), also in agreement with TEM images of untransfected cells (FIG. 16). In contrast, the RLC-QPM images obtained for the cell transfected according to the embodiments that have been given sufficient time to express RfA1 show: 1) slightly rounded cell morphologies (FIG. 23, right), in agreement with standard phase contrast microscopy imaging of transfected cells (FIG. 10, left); 2) more pronounced phase difference between the cells' bodies (now even darker gray areas) and the glass substrate (relatively light gray areas), with the cell bodies featuring a substantial number of regions with a higher phase difference (large dark black spots) that, not to be bound by any theory, likely correspond to reflectin-based structures, in agreement with TEM images of reflectin-expressing cells (FIGS. 13 and 14A-14B). Accordingly, in many embodiments, the way in which the transfected cells of the instant application reflect and scatter light is altered by the formation of disordered arrangements of reflectin biomolecule-based structures within the cells' interiors.

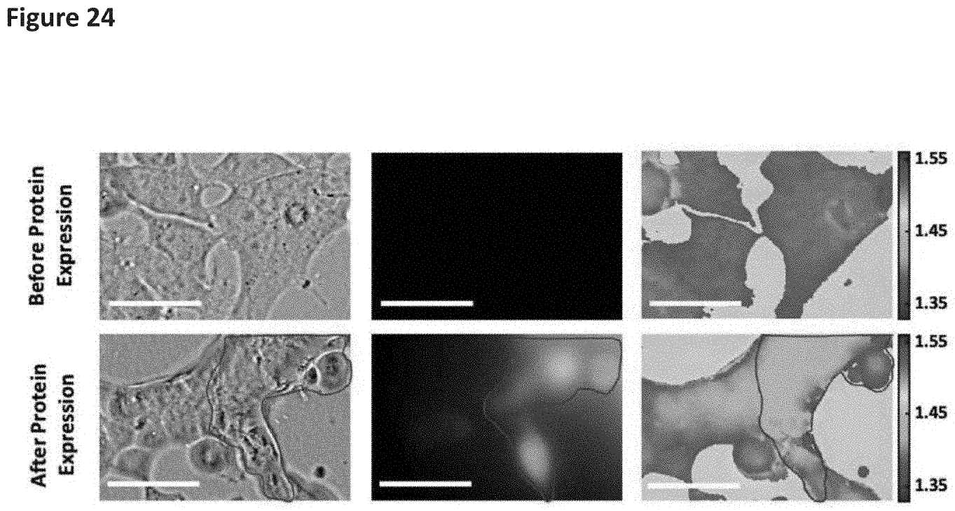

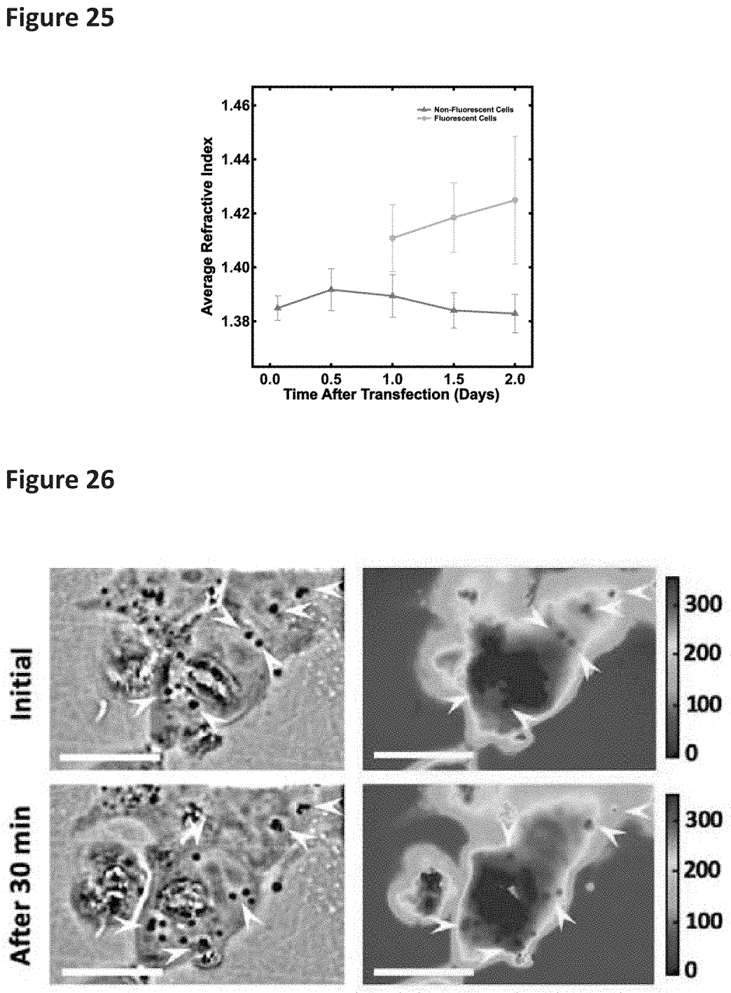

FIGS. 24 and 25 provide illustrative quantification of the effect that reflectin presence has on the optical characteristics (specifically, the refractive indices) of the transfected cells of the instant application. More specifically, FIG. 24 provides RLC-QPM and fluorescence microscopy visualization of HEK 293 cells transfected with a vector encoding for the expression of both histidine-tagged RfA1 and the RFP reporter according to many embodiments. Here, RLC-QPM facilitates recording of not only the phase images, but also the corresponding optical pathlengths and geometric heights for different cells before and after protein expression, subsequently enabling precise calculation and comparison of the cells' refractive index maps. In tandem, fluorescence microscopy provides verification of RfA1 expression via monitoring of the RFP reporter, making it possible to unambiguously differentiate between RfA1-expressing and untransfected cells. To this end, first, FIG. 24 (top left) provides an RLC-QPM image of recently-transfected cells, which are not yet expressing RfA1 and RFP, and shows that such cells feature a relatively minimal phase difference with the substrate. Furthermore, the simultaneous fluorescence microscopy image of the same cells provided in FIG. 24 (top middle) does not show any fluorescence signals associated with the RFP reporter. Finally, the corresponding cellular refractive index distribution for the same cells appears to be generally uniform with an average value of ˜1.38 (FIG. 24, top right), which matches literature precedent for whole mammalian cells (Rappaz, B. et al. Measurement of the integral refractive index and dynamic cell morphometry of living cells with digital holographic microscopy. Optics express 13, 9361-9373 (2005); Lue, N. et al. Live cell refractometry using microfluidic devices. Optics letters 31, 2759-2761 (2006), the disclosures of which is incorporated herein by reference). In contrast, the RLC-QPM images of the cells that have been given sufficient time to express RfA1 and RFP show, for example in FIG. 24 (bottom left), that some of such cells feature a more pronounced phase difference with the substrate, presumably (although not to be bound by any theory) due to the presence of reflectin-based structures inside the transfected cells. Furthermore, the fluorescence microscopy images of the same cells reveal that some of the cells exhibit clear RFP-associated fluorescence signals, confirming successful protein expression (FIG. 24, bottom middle). Finally, the corresponding cellular refractive index distributions are no longer uniform: the non-fluorescent cells, which have failed to express both RfA1 and RFP, retain an average refractive index of ˜1.38, whereas the fluorescent cells, which have successfully expressed both proteins, reach an average refractive index of >˜1.42 (FIG. 24, bottom right). Notably, FIG. 25 shows that, in general, the average refractive indices of the fluorescent cells, i.e., the reflectin producing cells of the many embodiments of the instant application, are consistently higher (remaining relatively unchanged after the emergence of fluorescence) than the average refractive indices of the control non-fluorescent, i.e., non-reflectin producing cells (which are also consistent and remain relatively unchanged for days). It should be noted, the RfA1- and RFP-expressing cells do exhibit some variability in their refractive index values, with more intense fluorescence signals and greater structure/aggregate volume fractions typically correlating to higher indices. Accordingly, in many embodiments, the optical properties, including refractive indices, of the cells of the instant application transfected to produce reflectin biomolecule can be engineered through the introduction of arrangements of reflectin biomolecule-based structures (i.e., the photonic architectures) into the cells' interiors.

FIG. 26 illustrates the influence of the reflectin protein-based structures on the local propagation of light through the transfected cells of many embodiments. More specifically, FIG. 26 provides images (left side) and the corresponding optical pathlength maps (right side) obtained from Transmission-Mode Low Coherence Quantitative Phase Microscopy (TLC-QPM) of RfA1-expressing HEK 293 cells taken in real time. TLC-QPM technique measures how incident light changes its phase when transmitted by different objects, such as living cells positioned on a substrate, and enables generation of both phase images and corresponding optical pathlength maps. Here, the TLC-QPM image obtained for the reflecting-expressing cells of many embodiments at a given time show numerous higher-phase structures (dark black spots, white arrows), which are distributed throughout the cells' interiors and possess apparent diameters on the order of ˜0.8 to 2 microns (FIG. 26, top left). The corresponding optical pathlength map provided in FIG. 26 (top, right) indicates that the longer pathlengths (with respect to the immediate surroundings) are exactly correlated to these structures' subcellular locations (dark red spots, white arrows), suggesting that their presence has substantially modified the local refractive index. Furthermore, the TLC-QPM images obtained for the same reflectin-expressing cells after a period of 30 minutes, provided, for example, in FIG. 26 (bottom left), show that the cells still contain numerous higher-phase structures (dark black spots, white arrows), wherein the structures have maintained their apparent sizes but changed their positions within the cells. The corresponding optical pathlength maps indicate that the longer pathlengths (again with respect to the immediate surroundings) have analogously shifted but are still correlated to the structures' subcellular locations (dark red spots, white arrows), suggesting a concomitant spatial redistribution of the modification in the local refractive index (FIG. 26, bottom right). Notably, the time-lapse videos generated from multiple phase images and optical pathlength maps collected for the cells at different time intervals demonstrate that the subcellular reflectin-based structures dynamically move within the cytoplasm and occasionally congregate near the membranes, positioning for release into the surroundings within extracellular vesicles. These observations, in general, agree with the TEM analysis presented in FIGS. 13 and 14A-14B, wherein the TEM images obtained for the reflectin-expressing cells of the instant application show the expulsion of the larger reflectin-based structures by the cells. Accordingly, in many embodiments, the local refractive indices and light-transmitting properties of the individual transfected cells of the instant application are dictated by the specific position and/or arrangement of the reflectin biomolecule-based structures (i.e., photonic architectures) found within the cells' interior.

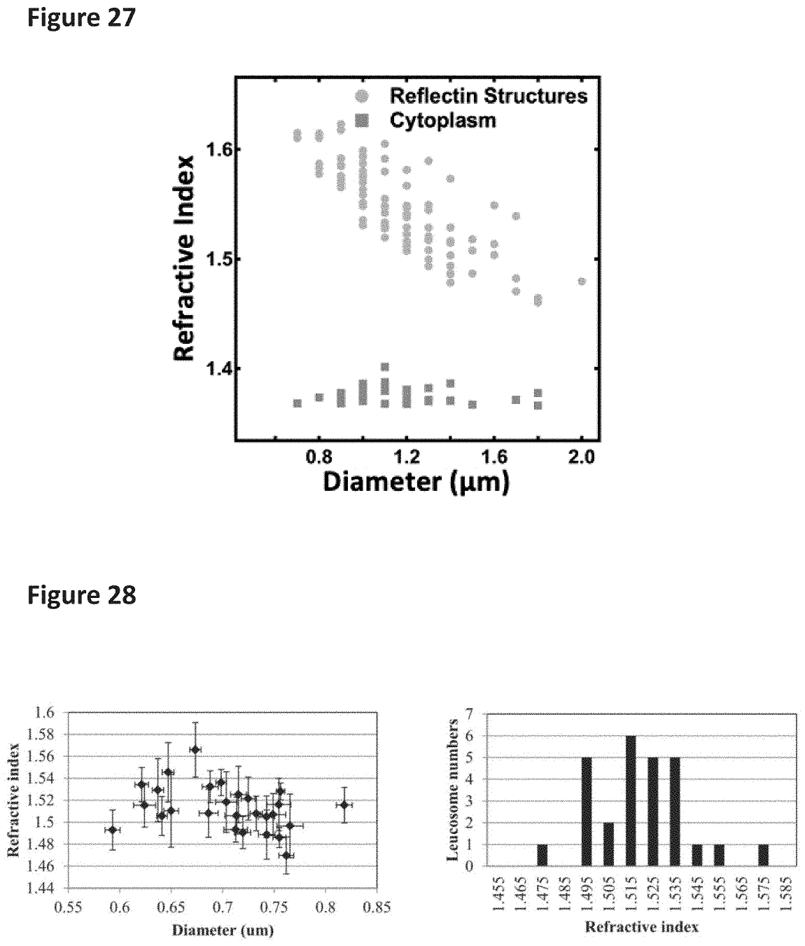

FIGS. 27 and 28 quantitatively illustrate the optical characteristics of the individual reflectin-based structures contained within the transfected cells of many embodiments. Specifically, FIG. 27 provides a plot of refractive index values vs. corresponding diameters calculated for readily-distinguishable reflectin structures positioned near the perimeters of the reflectin-expressing cells. For the purpose of these calculations, 1) it is assumed that, as roughly estimated from the TLC-QPM images, the diameters of the reflectin structures are similar to the cells' heights at peripheries; and 2) the equation used is

$$OPL = \frac{λΔϕ}{4 π} = d ( {n}_{a} - {n}_{s} ) ;$$

wherein OPL is the optical pathlength, λ is the central wavelength of the imaging light, Δϕ is the phase difference, d is the apparent diameter of the structure, nₐ is the refractive index of the structure, and nₛ is the refractive index of the immediate surroundings. The calculations performed for an ensemble of representative RfA1-based structures yield size-dependent refractive indices that vary from ˜1.40 to ˜1.62, with the higher and lower values generally corresponding to the smaller and larger apparent diameters, respectively (FIG. 27, and other studies according to embodiments). Notably, the refractive index values obtained for the reflectin structures found in the transfected cells of the instant application are comparable to the refractive indices of ˜1.44 reported for reflectin-filled condensed lamella in natural iridophores, ˜1.51 reported for reflectin-containing leucosomes in natural leucophores, and ˜1.54 to ˜1.59 reported for substrate-bound reflectin films. In contrast, performing the analogous calculations for representative regions of the cytoplasm proximal to the large reflectin structures of the application yields size-independent refractive index values that vary from ˜1.36 to ˜1.39 (FIG. 27), which are comparable to the refractive indices of ˜1.35 to ˜1.37 previously reported for the cytoplasm of mammalian cells (Choi, W. et al. Tomographic phase microscopy. Nat. Method 4 717-719 (2007); Liu, P. Y. et al. Cell refractive index for cell biology and disease diagnosis: past, present and future. Lab on a Chip 16, 634-644 (2016); Curl, C. L. et al. Refractive index measurement in viable cells using quantitative phase—amplitude microscopy and confocal microscopy. Cytometry Part A: the journal of the International Society for Analytical Cytology 65, 88-92 (2005), the disclosures of which is incorporated herein by reference). Moreover, the refractive index distribution observed for the larger reflectin-based structures found at the perimeters of the transfected cells of the application (FIG. 27) roughly resembles the refractive index distribution for the larger reflectin-containing leucosomes found inside cuttlefish leucophores (FIG. 28). Accordingly, in many embodiments, the optical properties of the transfected cells of the application, including their refractive index and overall opaqueness/transparency, is dynamically controllable via modifications to the number, size, arrangement, and cytoplasmic location of the reflectin biomolecule-based structures contained within the transfected cells. Furthermore, in some embodiments, the optical characteristics of the transfected cells of the application are engineered with high precision by engineering the cells to express reflectin biomolecules conjugated with one or more targeting biomolecules, e.g., peptides, of known conformation and aggregation behavior, and then inducing the resulting hybrids to form well-defined distributions of high refractive index aggregates in specific subcellular locations.

### Tunable Optical Properties of Transfected Cell Cultures

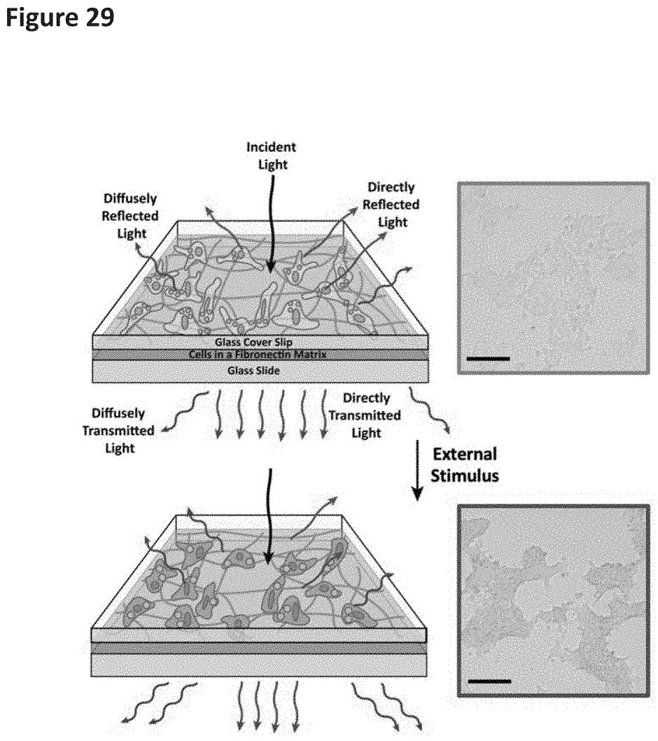

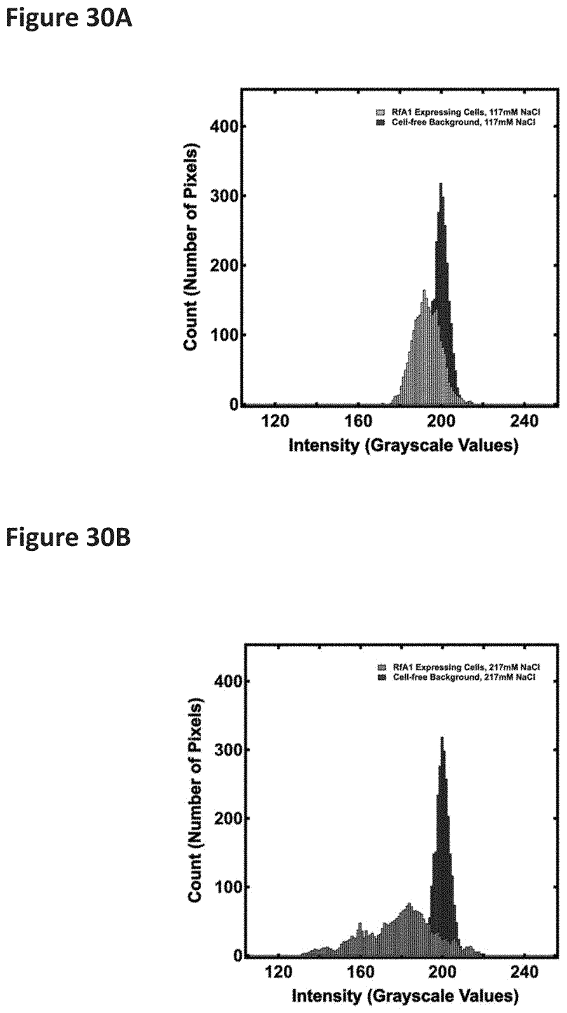

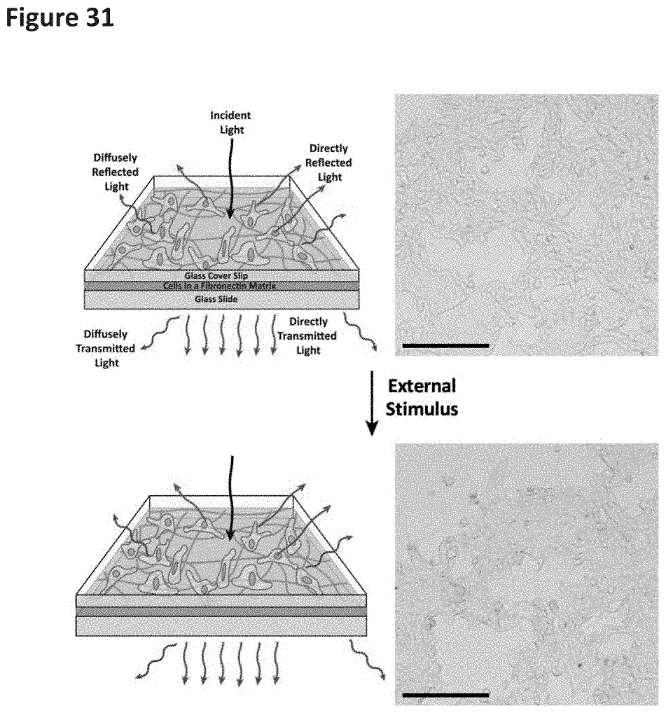

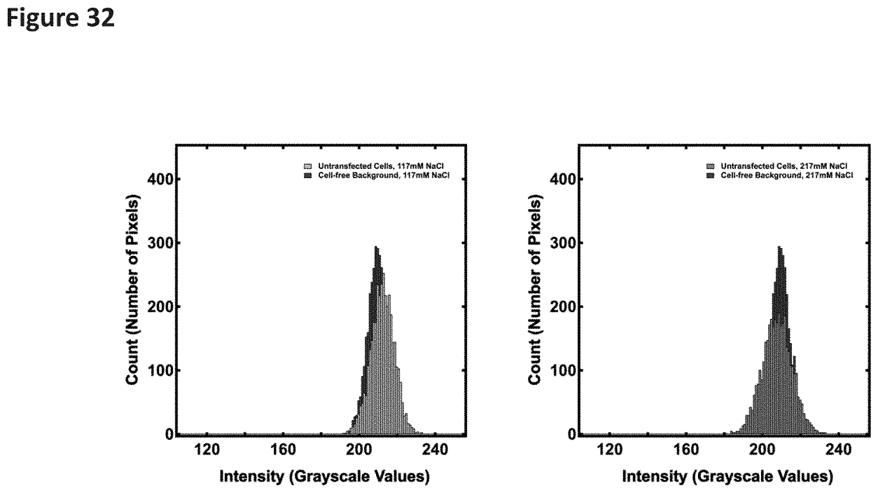

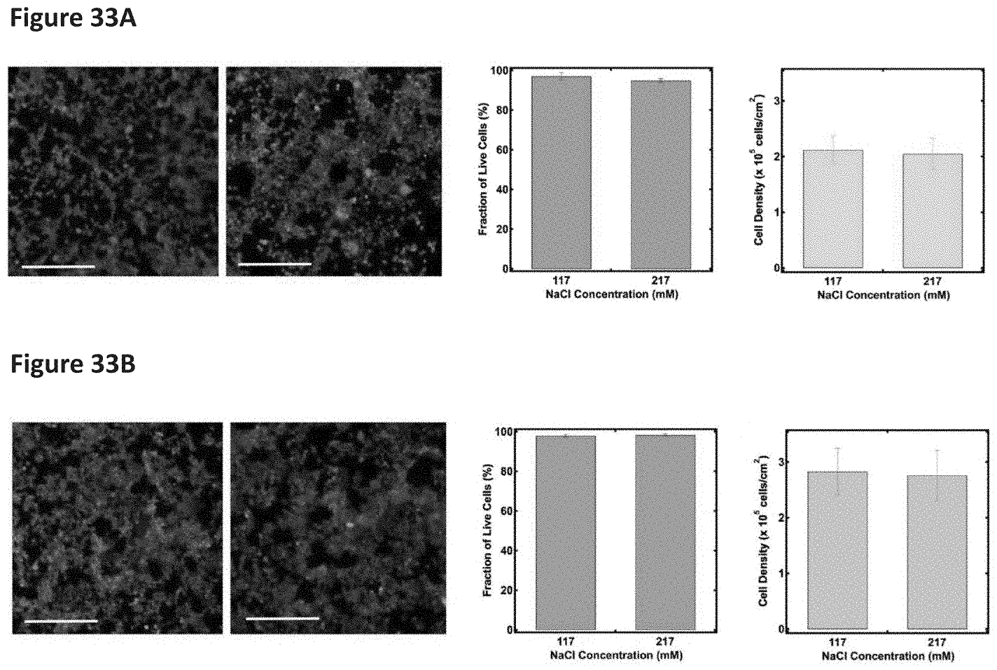

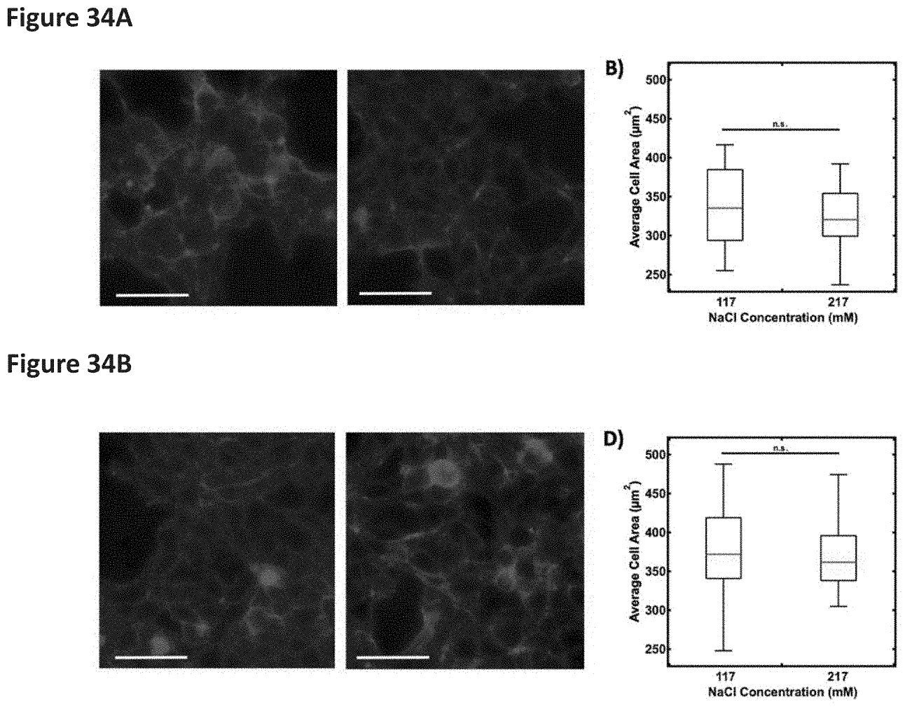

FIGS. 29-44 illustrate how the light-transmitting properties of the cell cultures transfected to produce reflectin according to many embodiments of the instant application can be controllably modulated (i.e., tuned) with an external chemical stimulus. More specifically, FIG. 29 shows an experimental setup, wherein brightfield microscopy is used to interrogate sandwich-type configurations, wherein: the bottom layer is comprised of a fibronectin-coated glass slide, the middle layer is comprised of fixed RfA1-expressing HEK 293 cells exposed to media with different ionic strengths (NaCl concentrations), and the top layer is comprised of a glass coverslip overlaid onto the cells. Here, brightfield microscopy is a technique which measures how incident light is attenuated upon transmission through different objects and furnishes corresponding brightness/intensity images that are readily analyzed via digital image processing methods (Bankman, I. N. (ed.) Handbook of Medical Image Processing and Analysis, Ch. 51 Pietka, E. (2009); Tan, L. et al. Digital Signal Processing (2013), the disclosures of which is incorporated herein by reference). Accordingly, the brightfield microscopy images obtained for the reflectin-expressing cell cultures of many embodiments exposed to media with a standard (i.e. 117 mM) NaCl concentration (FIG. 29, top right) show that the cells do not substantially attenuate the incident light and appear similar to the environment. However, the analogous reflectin-expressing cell cultures exposed to media with a higher (i.e. 217 mM) NaCl concentration attenuate the incident light more strongly and appear relatively distinct from the environment (FIG. 29, bottom right). Furthermore, FIGS. 30A and 30B provide representative histograms of the number of pixels at different intensity values (wherein, on this scale, 0 corresponds to black and 255 corresponds to white, so that a smaller number for the maximum implying a darker image) obtained from the brightfield microscopy images of the transfected cell cultures exposed to 117 mM and 217 mM NaCl media respectively. Accordingly, the histogram for the transfected cells exposed to the standard, 117 mM, NaCl concentration media, spans a relatively narrow range of ˜175 to ˜215, with a maximum at ˜195 (FIG. 30A), quantitatively confirming the cells' similarity to the surroundings, while the histogram for the transfected cells exposed to the higher, 217 mM, NaCl concentration media, spans a wider range of ˜130 to ˜220, with a shifted maximum at ˜187, further quantitatively confirming the cells' increased contrast with the surroundings (FIG. 30B). In addition, FIGS. 31 and 32 show control experiments for untransfected cells containing no reflectin that can be compared to transfected cells producing reflectin. First, the brightfield microscopy images obtained for the untransfected cell cultures demonstrate in FIG. 31 that cells without reflectin attenuate less incident light than the reflectin containing cells, and that such cells do not substantially change appearance with exposure to the higher NaCl concentration media. Second, the corresponding histograms obtained for the brightfield microscopy images of the untransfected cells and presented in FIG. 32 further demonstrate that the untransfected cells are almost indistinguishable from their surroundings, and that they remain relatively unchanged upon exposure to the higher NaCl concentration media. Finally, as additional controls, FIGS. 33A and 33B provide illustrative fluorescence microscopy images of live transfected (FIG. 33A) and untransfected (FIG. 33B) cells stained with the calcein AM and ethidium homodimer-1 dyes confirming that the cells' viabilities and densities are unaffected by the media's NaCl concentration, while FIGS. 34A and 34B provide illustrative fluorescence microscopy images of fixed transfected (FIG. 34A) and untransfected (FIG. 34B) cells stained with fluorophore-tagged wheat germ agglutinin, additionally confirming that the cells' areas are unaffected by the media's NaCl concentration. Accordingly, in many embodiments, the high refractive index, reflectin biomolecule-based photonic architectures internalized by the cells transfected according to the methods of the instant application determine the way in which the host cells transmit light. In many such embodiments, the exposure of the reflectin biomolecule hosting cells of the instant application to media of variable ionic strength alters (tunes) their light-transmitting properties, including transparency with respect to the surrounding, without influencing the cells' viabilities or morphologies.

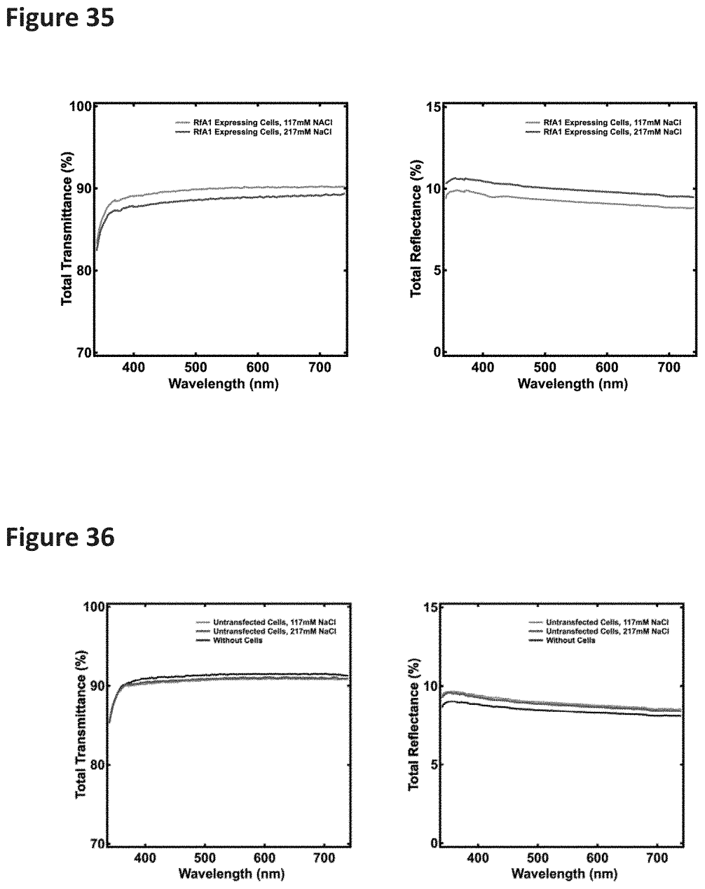

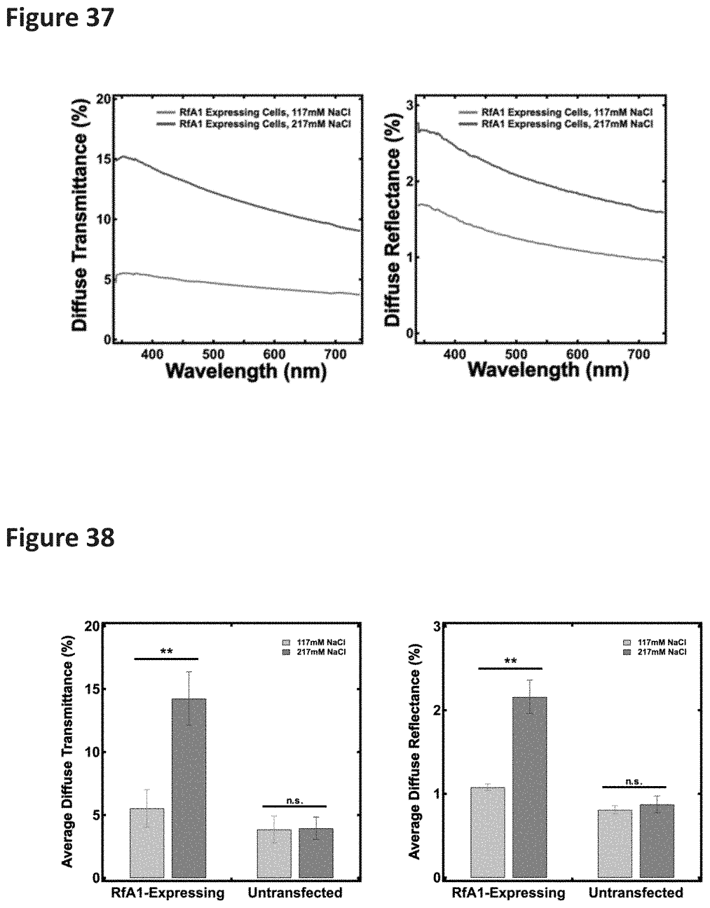

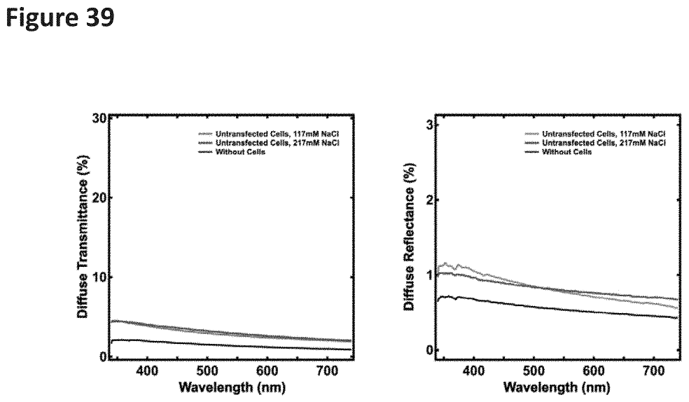

FIGS. 35-39 illustrate the effect of the application of a chemical stimulus, such as media with variable NaCl concentrations, on the transmission and reflection of light by the transfected cell cultures of the instant application. To this end, first, FIG. 35 provides the total transmittance and total reflectance spectra recorded for the sandwich-type configurations prepared from RfA1-expressing cells, as described above and depicted in FIG. 29, that have been exposed to media with different ionic strengths (NaCl concentrations). Accordingly, the illustrative spectra in FIG. 35 indicate that the cells' broadband transmittance decreases, while their broadband reflectance increases, upon exposure to the higher NaCl concentration media. For comparison, FIG. 36 provides the total transmittance and reflectance spectra recorded for the untransfected cell cultures and shows that the untransfected cells' broadband transmittance and reflectance are higher and lower, respectively, than those of the reflectin-expressing cell cultures, and that these spectra remain relatively unchanged by exposure to the higher NaCl concentration media. Next, FIG. 37 provides diffuse transmittance and diffuse reflectance spectra recorded for the RfA1-expressing cell cultures exposed to media with different ionic strengths, while FIG. 38 provides quantitative analysis thereof. Together, this illustrative data shows that the exposure of the transfected cells of the instant application to the higher NaCl concentration media increases the diffuse components of the transmittance from ˜5.5 (±1.5) % to ˜13 (±2.1) % (i.e., by >˜2-fold) and the diffuse components of the reflectance from ˜1.1 (±0.1) % to ˜2.1 (±0.2) % (i.e., by ˜2-fold). In contrast, the diffuse transmittance and diffuse reflectance spectra recorded for the untransfected cell cultures reveal the diffuse reflectance and transmittance components that are both lower than those of the reflectin-expressing cell cultures, and also, demonstrate that the optical properties of the untransfected cells remain relatively unchanged by the exposure to the higher NaCl concentration media (FIGS. 38 and 39). In addition, as further controls, the transmittance and reflectance spectra obtained for fibronectin matrices without any cells show relatively high total transmittances and low total reflectances with minor background diffuse components (FIGS. 36 and 39). Notably, the NaCl concentration-induced tuning of the transparency and broadband diffuse reflectance for the sandwich-type configurations prepared with reflectin-expressing cells of the instant application (FIGS. 29 and 37) bear a superficial resemblance to acetylcholine-triggered switching of the opacity and broadband reflectance for female D. opalescens squid's leucophore-containing layers (FIGS. 1A-1C). Accordingly, in many embodiments, the introduction of high refractive index, reflectin biomolecule-based photonic architectures causes the transfected host cells to diffusely transmit and or diffusely reflect (i.e., scatter) more of the incident visible light. In many such embodiments, the exposure of the reflectin biomolecule-expressing cells to media of variable ionic strength further alters (tunes) their scattering of visible light and their ability to change transparency with respect to the surroundings. In many embodiments, the transfected cells of the application can emulate some of the camouflage capabilities of squid skin tissues.

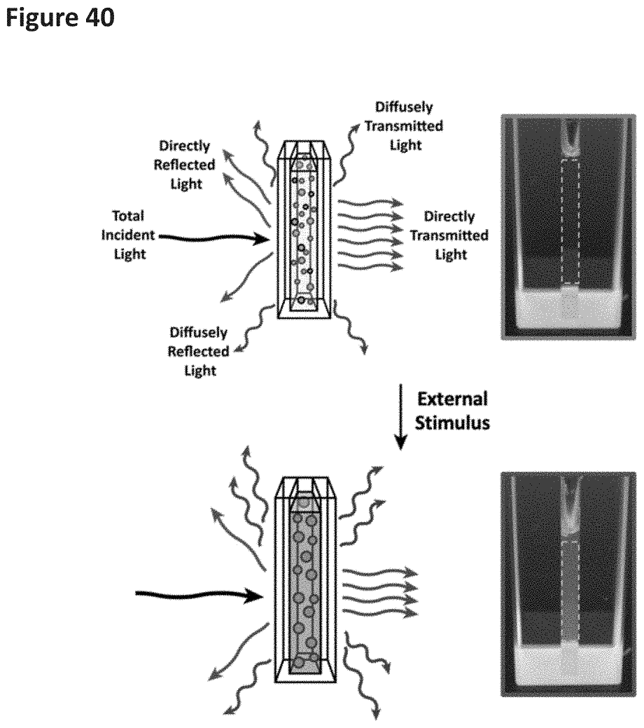

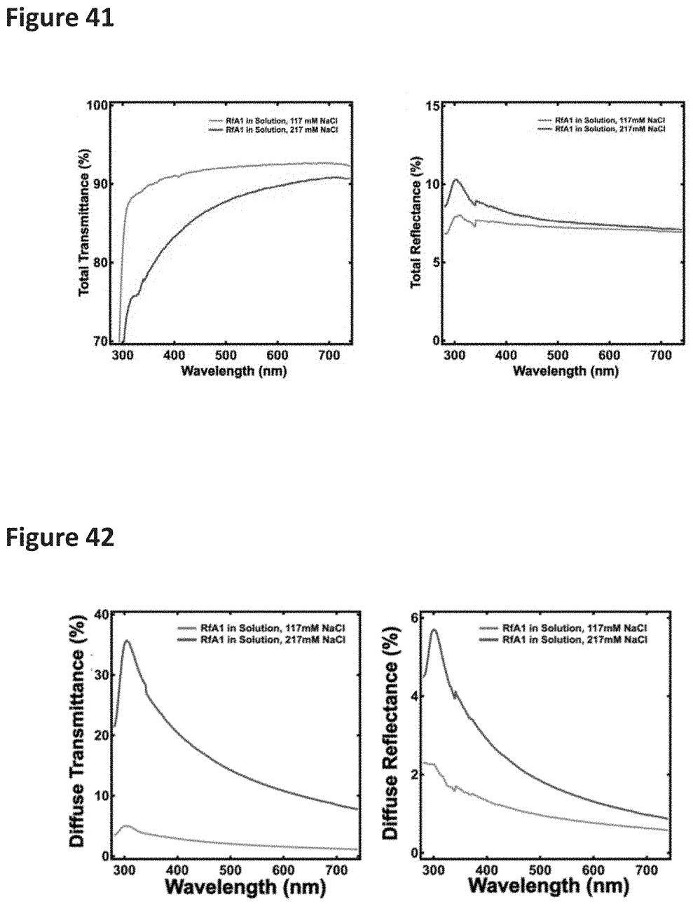

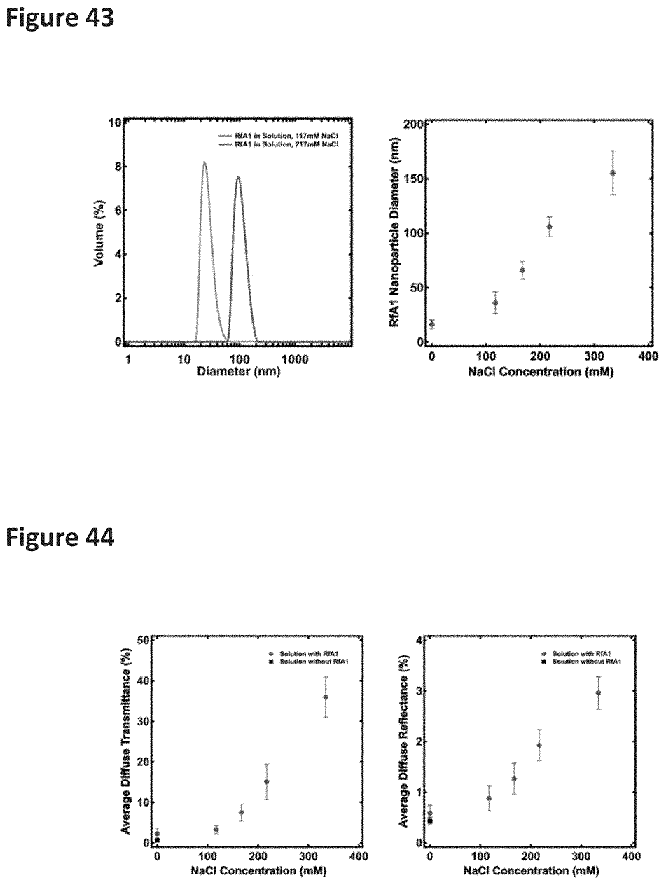

FIGS. 40-44 further illustrate the mechanistic underpinnings of tunable scattering of incident light by the transfected cells of the instant application. More specifically, these figures illustrate how a chosen chemical stimulus (in this case NaCl concentration) influences the appearances, transmittances, and reflectances of the aqueous reflectin solutions prepared as in vitro model systems for the reflectin-expressing cells of many embodiments. First, FIG. 40 illustrates the overall experimental set-up (left side) and provides digital camera images of the reflectin solution (prepared by solubilizing histidine-tagged RfA1 that has been heterologously expressed in E. coli) with systematically increased ionic strength (NaCl concentration). In addition, FIGS. 41, 42, and, 43 provide, correspondingly, the total transmittance and total reflectance spectroscopy analysis, the diffuse transmittance and diffuse reflectance analysis, and the dynamic light scattering (DLS) analysis of the same. Accordingly, the illustrative digital camera images (FIG. 40, top right) and total transmittance (FIG. 41, left) and total reflectance (FIG. 41, right) spectra obtained for RfA1 solutions with a standard (i.e. 117 mM) NaCl concentration show that the solutions appear visibly transparent to the naked eye, transmit most of the incident light, and reflect little of the incident light. In contrast, the digital camera images (FIG. 40, bottom right) and the corresponding total transmittance (FIG. 41, left), and total reflectance (FIG. 41, right) spectra obtained for RfA1 solutions with a high (i.e. 217 mM) NaCl concentration show that the solutions appear visibly opaque to the naked eye, transmit much less of the incident light, and reflect slightly more of the incident light. Furthermore, the diffuse transmittance (FIGS. 42, left) and diffuse reflectance (FIGS. 42, right) spectra, together with DLS analysis (FIG. 43), obtained for the solutions with the standard NaCl concentration reveal that such solutions feature relatively low diffuse transmittance and diffuse reflectance components with average values of ˜3.3 (±1.0) % and ˜0.8 (±0.2) %, respectively, and primarily contain reflectin structures with diameters of ˜36 (±10) nm, indicating that such solutions scatter visible light very weakly. In contrast, the same analysis of the solutions with the higher, 217 mM, NaCl concentration (FIGS. 42 and 43) demonstrates that these solutions feature much larger diffuse transmittance and diffuse reflectance components with average values of ˜15.1 (±4.4) % and ˜1.9 (±0.3) %, respectively, and primarily contain reflectin structures with diameters of ˜106 (±9) nm, indicating that such solutions scatter visible light more strongly, presumably (although not to be bound by any theory) via a Mie-type mechanism (Bohren, C. F. et al. Absorption and Scattering of Light by Small Particles (Wiley-VCH, Weinheim, Germany, 1998), the disclosure of which is incorporated herein by reference). In general, as can be seen from FIG. 43 (right) and 44, for the aqueous reflectin solution, the values of their diffuse transmittance and diffuse reflectance components, as well as the diameters of their constituent particles, steadily increase as a function of the NaCl concentration, underscoring the fact that the sizes and/or aggregation states of the reflectin-based structures determine the degree of light scattering and overall transparencies of the solutions. Moreover, FIG. 44 also shows that in the absence of reflectin, the same aqueous solutions have relatively low background diffuse transmittances and diffuse reflectances, and are completely transparent to the naked eye, further supporting the importance of the reflectin-based structures of many embodiments. Accordingly, in many embodiments, the changes in the external ionic strength reconfigures the geometries and or arrangements of the high refractive index, reflectin biomolecule-based photonic architectures contained within the transfected cells of the instant application, as such, modulates the host cells' diffuse transmission and/or scattering of incident visible light. In many such embodiments, the reflectin biomolecule-based architectures found within the transfected cells of the instant application are targeted with external stimulus or stimuli according to Mie-type mechanism to impart adaptive transparency or other optical capabilities to the host cells.

Accordingly, in many embodiments, living biological cells are designed and engineered to comprise reflectin biomolecules and/or reflectin biomolecule-based photonic architectures, which enable the host cells to adaptively scatter light in a fashion similar to cephalopods. Therefore, in many embodiments, the reflectin biomolecule-containing cells of the application provide model systems that overcome challenges associated with culturing of cephalopod skin cells and allow for studies of cephalopod functionalities that critically rely upon reflectins, as well as studies to gain a better fundamental understanding of molluscan molecular and cellular biology in general. Furthermore, in many embodiments, the reflectin biomolecule-based structures formed within the transfected cells of the instant application are relied upon, due to their intrinsically high refractive indices and unique amino acid sequences, to serve as biomolecular reporters for the broadly-applicable visualization and quantitation of dynamic cellular processes across many different mammalian species via ubiquitous phase contrast microscopy, in approaches reminiscent of the ones pioneered for jellyfish green fluorescent proteins with fluorescence microscopy (Tsien, R. Y. The green fluorescent protein. Annu. Rev. Biochem. 67, 509-544 (1998); Chudakov, D. M., et al. Fluorescent proteins and their applications in imaging living cells and tissues. Physiol Rev. 90, 1103-1163 (2010), the disclosures of which is incorporated herein by reference). Moreover, in some embodiments, the reflectin biomolecule-expressing cells of the instant application, enabled by the reflectins' diverse self-assembly properties and ease of expression, are engineered to facilitate genetically-encoded refractive index matching within entire organisms and, thus, to allow scientists to image living specimens in real time with improved clarity and resolution on conventional optical microscopes, by analogy with studies of deceased cells and tissues with clearing techniques. In addition, in some embodiments, the reflectin biomolecule-expressing cells of the instant application are engineered for in vivo formation of metamaterial-like photonic architectures, and, as such, enable development of human cells and tissues with unprecedented adaptive appearance-changing capabilities, such as stimuli-responsive iridescence or mechanically-reconfigurable coloration. Furthermore, in many embodiments, the transfected cells of the instant application facilitate and enable studies as diverse as, for example, general three-dimensional inter- and intra-cellular organization, phase separation and phase transformations of disordered proteins, the formation of membrane-less and membrane-bound organelles, and the evolution of proteinaceous aggregates commonly associated with neurodegenerative diseases.

### EXEMPLARY EMBODIMENTS

The following examples are put forth so as to provide those of ordinary skill in the art with a complete disclosure and description of how to make and use the present invention, and are not intended to limit the scope of what the inventors regard as their invention, nor are they intended to represent that the experiments below are all or the only experiments performed. Efforts have been made to ensure accuracy with respect to numbers used (e.g., amounts, temperature, etc.), but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., s or sec, second(s); min, minute(s); h or hr, hour(s); and the like.

### Example 1—Growth and Transfection of Human Cells

The human embryonic kidney (HEK) 293 cells were grown and transfected according to standard protocols. First, vector constructs encoding for the independent expression of N-terminal histidine-tagged Doryteuthis (Loligo) pealeii reflectin A1 (RfA1) (Genbank: ACZ57764.1), the independent expression of Cayenne Red Fluorescent Protein (RFP), or the co-expression of both RfA1 and RFP (with the expression of the latter mediated by an internal ribosome entry site from the encephalomyocarditis virus) were designed by ATUM using their Gene Designer Software. The vectors all contained 5′UTR regions downstream of a cytomegalovirus promoter and enhancer, a standard origin of replication derived from pBR322, a polyadenylation signal to aid in the termination of transcription, and cDNA encoding for the protein or proteins of interest. Next, HEK 293 cells (ATCC, CRL-1573™) were cultured on plastic or fibronectin-coated glass dishes in Minimal Essential Medium (MEM) supplemented with Earle's salts and 10% fetal bovine serum (FBS) (Life Technologies) at a temperature of 37° C. and under 5% CO₂. For transfection, the HEK 293 cells were seeded at ˜5% to ˜33% of the confluent density for the plastic or glass dishes and grown for another ˜14 to ˜24 h. The medium was swapped for MEM supplemented with Earle's salts but lacking FBS. A transfection reagent mixture containing Lipofectamine 2000 (Life Technologies) and a vector encoding for just RfA1, just RFP, or both RFA1 and RFP (ATUM) was added to the medium, and the cells were incubated for ˜24 to ˜48 h. The untransfected or transfected cells were fixed as necessary, used for the preparation of cross-sections, or directly characterized with microscopy techniques.

### Example 2—Expression and Purification of N-Terminal Histidine-Tagged Reflectin A1 in Bacteria

N-terminal histidine-tagged RfA1 was expressed and purified according to procedures reported in the literature. In brief, an E. coli codon optimized gene coding for the histidine-tagged RfA1 protein from Doryteuthis (Loligo) pealeii (Genbank: ACZ57764.1) was synthesized and cloned into the pJExpress414 vector (ATUM). This expression vector was transformed into BL21 (DE3) cells (Novagen). The protein was expressed in Lysogeny Broth (LB) (Novagen) supplemented with 100 μg/mL Carbenicillin at a temperature of 37° C. RfA1 was completely insoluble when expressed at 37° C. and was sequestered in inclusion bodies. The cells were lysed using BugBuster (Novagen), according to manufacturer protocols, and the inclusion bodies were extracted by filtration and centrifugation. The inclusion bodies were subsequently solubilized in denaturing buffer (6 M guanidine hydrochloride), and the protein was purified via high performance liquid chromatography (HPLC) on an Agilent 1260 Infinity system using a reverse phase C18 column. For purification, the gradient was evolved from 95% Buffer A:5% Buffer B to 5% Buffer A:95% Buffer B at a flow rate of 4 mL/min over 35 min (Buffer A: 99.9% water, 0.1% trifluoroacetic acid; Buffer B: 95% acetonitrile, 4.9% water, 0.1% trifluoroacetic acid). The pure RfA1 was collected, flash frozen in liquid nitrogen, and lyophilized. The identity of the protein was confirmed with sodium dodecyl sulfate polyacrylamide gel electrophoresis (SDS-PAGE), tryptic digestion, and mass spectrometry, prior to use in other biophysical characterization experiments.

### Example 3—Preparation of Human Cells for Immunofluorescence Microscopy

The untransfected or transfected HEK 293 cells were fixed and labeled with fluorescent markers according to standard protocols. First, the cells were seeded on 8-well or 12-well glass bottom micro-slides (Ibidi) coated with human fibronectin (Corning) at a density of ˜30,000 cells/cm² and were grown for ˜14 to ˜16 h. When necessary, the HEK 293 cells were either 1) transfected with vectors encoding for just RfA1, just RFP, or both RfA1 and RFP for ˜48 h (see Example 1 above); 2) subjected to the transfection reagents in the absence of any vector, i.e. “mock” transfected, under the same conditions; or 3) subjected to the FBS-free growth media in the absence of any transfection reagents or vectors under the same conditions. The untransfected or transfected cells were fixed with 3% paraformaldehyde (PFA) in 0.1 M phosphate buffer (PB), permeabilized with 0.1% Triton-X 100 in phosphate buffered saline (PBS) containing 1% bovine serum albumin (BSA), and blocked with 1% BSA in PBS. The fixed untransfected or transfected cells were incubated with either an oligoclonal rabbit anti-histidine tag primary antibody (ThermoScientific) solution (prepared at a ratio of 1:500 in PBS containing 1% BSA) or a polyclonal rabbit anti-reflectin primary antibody solution (prepared at a ratio of 1:1000 prepared PBS containing 1% BSA). The cells were thoroughly washed with PBS and incubated with a goat anti-rabbit IgG Alexa 488 secondary antibody (ThermoScientific) solution (prepared at a ratio of 1:250 in PBS containing 1% BSA) and with the nuclear stain 4′,6′-diamidino-2-phenylindole (DAPI) (ThermoScientific). After labeling, the cells were again washed with PBS, and treated with anti-fade mounting media (Ibidi). The resulting stained fixed untransfected and transfected cells were imaged with confocal fluorescence microscopy.

### Example 4—Preparation of Human Cells for Live Dead Assays

The untransfected or transfected HEK 293 cells were labeled with the Calcein AM dye (live cell stain) and the Ethidium Homodimer-1 dye (dead cell stain) according to standard protocols. First, the cells were seeded on 3-well removable chamber glass slides (Ibidi) coated with human fibronectin (Corning) at a density of ˜60,000 cells/cm² and were grown for ˜14 to 16 h. When necessary, the HEK 293 cells were transfected with vectors encoding for RfA1 for 48 h (see Example 1 above) or were subjected to the FBS-free growth media in the absence of any transfection reagents or vectors under the same conditions. Next, the untransfected or transfected cells were incubated for ˜1 h in MEM supplemented with Earle's salts, for which the NaCl concentration was adjusted to 117 mM or 217 mM. In turn, the cells were washed with D-PBS (ThermoScientific) and stained with Calcein AM (ThermoScientific) and Ethidium Homodimer-1 (ThermoScientific). The resulting stained untransfected and transfected cells were imaged with fluorescence microscopy.

### Example 5—Preparation of Human Cells for Cell Area Assays

The untransfected or transfected HEK 293 cells were labeled with fluorescently-labeled wheat germ agglutinin according to standard protocols. First, the cells were seeded on 3-well removable chamber glass slides (Ibidi) coated with human fibronectin (Corning) at a density of 60,000 cells/cm² and were grown for ˜14 to ˜16 h. When necessary, the HEK 293 cells were transfected with vectors encoding for RfA1 for ˜48 h (see Example 1 above) or were subjected to the FBS-free growth media in the absence of any transfection reagents or vectors under the same conditions. Next, the untransfected or transfected cells were incubated for ˜1 h in MEM supplemented with Earle's salts, for which the NaCl concentration was adjusted to 117 mM or 217 mM. In turn, the cells were stained with Alexa 555 fluorophore-conjugated wheat germ agglutinin (ThermoScientific) in Hank's Balanced Salt Solution (HBSS) (ThermoScientific) and subsequently washed in pre-warmed HBSS. Finally, the cells were fixed in 3% PFA in 0.1 M PB. The resulting stained untransfected and transfected cells were imaged with fluorescence microscopy.

### Example 6—Preparation of Human Cell Cultures for Brightfield Optical Microscopy and Reflectance and Transmittance Spectroscopy

The untransfected or transfected HEK 293 cell cultures were integrated into sandwich-type configurations. First, 3-well removable chamber glass slides (Ibidi) were coated with human fibronectin (Corning). Next, HEK 293 cells were seeded at densities of ˜60,000 cells/cm² and were grown for ˜14 to ˜16 h. When necessary, the cells were transfected with vectors encoding for RfA1 for ˜48 h (see Example 1 above) or were subjected to the FBS-free growth media in the absence of any transfection reagents or vectors under the same conditions. Next, the untransfected or transfected cells were incubated for ˜1 h in MEM supplemented with Earle's salts, for which the NaCl concentration was adjusted to 117 mM or 217 mM. In turn, the substrates with monolayers at a ˜50% to ˜75% confluency were fixed with 3% PFA in PBS, thoroughly washed with PBS, treated with anti-fade mounting media (Ibidi), and covered (overlaid) with a thin glass coverslip. Note that the preparation and use of cell cultures within the above confluency window ensured rigorous quality control and facilitated comparisons across all of the experiments. The resulting configurations, which contained either fixed transfected or untransfected cells, were imaged with brightfield optical microscopy and characterized with reflectance and transmittance spectroscopy.

### Example 7—Preparation of Cellular Cross-Sections for Transmission Electron Microscopy

The untransfected or transfected HEK 293 were segmented into cross-sections according to literature protocols (see, for example, Spur, A. R. A low-viscosity epoxy resin embedding medium for electron microscopy. J. Ultrastruct. Res. 26, 31-43 (1969), the disclosure of which is incorporated herein by reference). First, HEK 293 cells were seeded at densities of 32,000 cells/cm² into T-25 flasks (ThermoScientific) and were grown for ˜18 to ˜24 h. When necessary, the HEK 293 cells were transfected with vectors encoding for just RfA1, just RFP, or both RfA1 and RFP for ˜48 h (see Example 1 above) or were subjected to the FBS-free growth media in the absence of any transfection reagents or vectors under the same conditions. Alternatively, to obtain independent confirmation of our experiments, the HEK 293 cells were cultured in Improved MEM supplemented with 10% FBS and were transfected with a reagent mixture containing Fugene HD and the vector encoding for RfA1 at ATUM. Next, the cells were fixed with 2.5% glutaraldehyde in 0.1 M sodium cacodylate buffer (Electron Microscopy Sciences) and spun down into a cell pellet. Subsequently, the pellet was blocked with 1% osmium tetroxide in 0.15 M sodium cacodylate buffer (LADD research), stained with 2% uranyl acetate in double distilled water (LADD Research), and dehydrated with ethanol (LADD research). The cells were then embedded in Durcupan resin (Sigma) and sectioned on an Ultracut UC6 Ultramicrotome (Leica) by using a diamond knife (Diatome). The sections were next transferred onto copper mesh grids (LADD research) and post-stained with uranyl acetate and lead citrate (Electron Microscopy Sciences). The final fixed, resin-embedded, grid-mounted cross-sections were imaged with transmission electron microscopy.

### Example 8—Preparation of Cellular Cross-Sections for Immuno-Electron Microscopy

The transfected HEK 293 cells were segmented and labeled with gold nanoparticles according to literature protocols (see, for example: Liou, W., et al. Improving structural integrity of cryosections for immunogold labeling. Histochem. Cell Biol. 106, 41-58 (1996); and Tokuyasu, K. T., Immunochemistry on ultrathin frozen sections. Histochem. J. 12, 381-403 (1980), the disclosures of which is incorporated herein by reference). First, HEK 293 cells were seeded at densities of ˜32,000 cells/cm² into T-25 flasks (ThermoScientific) and were grown for ˜18 to 24 h. The HEK 293 cells were then transfected with vectors encoding for both RfA1 and RFP (see Example 1 above) for ˜48 h. Next, the cells were fixed overnight using 4% PFA in 0.1 M PB (Electron Microscopy Sciences), rinsed with 0.15% glycine in 0.1 M PB, pelleted in 10% gelatin (Knox) in 0.1 M PB, and cryoprotected by infusion with 2.3 M sucrose in 0.1 M PB. The cell blocks of 1 mm³ were mounted onto cryopins, and flash frozen in liquid nitrogen. The resulting frozen blocks were cut into ˜70 to ˜90 nm ultrathin cross-sections at a temperature of −100° C. on an Ultracut UC6 Ultramicrotome with a cryo-attachment (Leica) by using a diamond cryo-knife (Diatome). The sections were in turn picked up with a 1:1 mixture of 2.3 M sucrose in 0.1 M PB and 2% methyl cellulose (Aldrich) in water and transferred onto Formvar and carbon-coated copper grids (Electron Microscopy Sciences). The grid-mounted sections were then placed on 2% gelatin in PBS, rinsed with 0.15% glycine in PBS, and blocked with 1% fish-skin gelatin (Sigma) in PBS. The grid-mounted sections were incubated with a polyclonal rabbit anti-reflectin primary antibody followed by a goat anti-rabbit secondary IgG conjugated to a 12 nm gold nanoparticle (Jackson Immuno Research). The resulting grid-mounted sections were post-fixed with 1% glutaraldehyde in PBS, washed thoroughly with distilled water, mounted, and subsequently post-stained with 0.2% uranyl acetate (LADD Research) in 1.8% methyl cellulose in water. The final fixed, resin-embedded, cryo-protected, and labeled cross-sections were imaged with a transmission electron microscope.

### Example 9—Preparation of Human Cells for Low-Coherence Quantitative Phase Contrast Microscopy

The untransfected and transfected HEK 293 cells were grown and transfected as described above, with minor modifications to the protocol. In brief, the cells were seeded at a density of ˜5,000 cells/cm² on glass substrates and grown for ˜14 to ˜16 h. Next, the cells were transfected with vectors encoding for just RfA1 or both RfA1 and RFP (see Example 1 above) immediately prior to imaging. For reflection-mode experiments, the cells were cultured on custom-designed 35 mm glass-bottom dishes featuring an anti-reflection coating, which were coated with human fibronectin (Corning). For transmission-mode experiments, the cells were cultured on custom-designed 35 mm glass-bottom dishes featuring a half-mirror coating, which were coated with human fibronectin. The untransfected or transfected cells were characterized with low-coherence quantitative phase contrast microscopy with or without a fluorescence microscopy attachment.

### Example 10—Preparation of Aqueous Reflectin A1 Solutions

The solutions were prepared according to procedures adopted from the literature. In brief, purified, lyophilized protein was first solubilized in deionized water at a concentration of 1 to ˜4 mg/mL and a low pH of <˜5. The protein concentration was then diluted to ˜0.5 mg/mL, while the NaCl concentration was adjusted to 117 mM, 167 mM, 217 mM, or 334 mM. The resulting solutions were characterized with transmittance and reflectance spectroscopy and dynamic light scattering.

### Example 11—Phase Contrast and Fluorescence Microscopy of Live Human Cells

The live untransfected or transfected HEK 293 cells were characterized with an Olympus IX51 equipped with an Olympus TH4100 light source, an Olympus U-RFL-T fluorescence laser source, and a QICAM camera. The resulting phase contrast and fluorescence images were analyzed with ImageJ.

### Example 12—Confocal Microscopy of Fixed and Immunolabeled Human Cells

The fixed, immunolabeled, untransfected or transfected HEK 293 cells were characterized with an LSM 780 confocal microscope equipped with a Nikon GaAsP detector and an Argon laser (with fluorophore excitation wavelengths of 405 nm, 458 nm, and 514 nm). The resulting confocal and fluorescence microscopy images were analyzed with ImageJ.

### Example 13—Fluorescence Microscopy of Stained Live and Fixed Human Cells

The untransfected or transfected HEK 293 cells stained with the Calcein AM and Ethidium Homodimer-1 dyes were characterized with an EVOS M5000 Imaging System (ThermoScientific) in fluorescence imaging mode. The fixed untransfected or transfected HEK 293 cells labeled with wheat germ agglutinin conjugated to an Alexa Fluor 555 dye were characterized with an EVOS M5000 Imaging System (ThermoScientific) in fluorescence imaging mode. The resulting images were analyzed with ImageJ.

### Example 14—Transmission Electron Microscopy of Cellular Cross-Sections

The fixed, resin-embedded, cross-sections obtained from untransfected or transfected HEK 293 cells were characterized with a Tecnai G2 Spirit BioTWIN transmission electron microscope equipped with an Eagle 4k HS digital camera (FEI). The resulting transmission electron microscopy images were analyzed with ImageJ.

### Example 15—Immuno-Electron Microscopy of Labeled Cellular Cross-Sections

The fixed, resin-embedded, cryo-protected, and labeled cross-sections obtained from transfected HEK 293 cells were characterized with a JEOL 1400Plus transmission electron microscope (JEOL) and outfitted with a OneView 16-megapixel digital camera (Gatan).

### Example 16—Low-Coherence Quantitative Phase Microscopy of Live Human Cells

The live transfected HEK 293 cells were characterized with a custom-built low-coherence quantitative phase contrast microscope (Hamamatsu). For reflection-mode experiments, the instrument was outfitted with a heating element, a piezo-driven adjustable sample stage (NanoControl), a fluorescence detection module featuring an excitation filter with a center wavelength of 525 nm (Edmund Optics), a high performance long-pass emission filter with a cut-on wavelength of 575 nm (Edmund Optics), and a light emitting diode with a broadband emission wavelength from 575 nm to 700 nm. The resulting interference images were analyzed and converted to optical pathlength maps, geometric height maps, and refractive index maps with MATLAB 2017a (MathWorks, Inc.) as previously described. For transmission-mode experiments, the instrument was outfitted with a heating element, an adjustable sample stage (OptoSigma), and a narrow band light emitting diode with an emission wavelength of 633 nm. The resulting interferences images were analyzed with MATLAB 2017a (MathWorks, Inc.) and converted to phase images and optical pathlength maps as previously described. The phase images and optical pathlength maps were further analyzed with ImageJ to extract the apparent diameter and refractive index of the RfA1-based structures or cytoplasmic regions.

### Example 17—Brightfield Optical Microscopy of Fixed Human Cells

The sandwich-type configurations from untransfected or transfected HEK 293 cells were characterized with an EVOS M5000 Imaging System (ThermoScientific) in brightfield imaging mode. The resulting brightfield optical images were analyzed with ImageJ. The histograms of the number of pixels at different intensity values were extracted from the images and analyzed by using the “Histogram” function in ImageJ according to standard image processing and analysis procedures reported in the literature (see, among others, Weber, A., et al. Polarimetric imaging and blood vessel quantification. Opt. Express 12, 5178-5190 (2004), the disclosures of which is incorporated herein by reference).

### Example 18—Transmittance and Reflectance Spectroscopy of Fixed Human Cells and Reflectin A1 Solutions

The sandwich-type configurations containing untransfected or transfected HEK 293 cells, and the RfA1 solutions in quartz cuvettes (Millipore Sigma) were characterized with a V-670 UV-VIS-NIR Spectrophotometer (Jasco) outfitted with a 150 mm Integrating Sphere (Jasco).

### Example 19—Dynamic Light Scattering Measurements for Reflectin A1 Solutions

The RfA1 solutions were characterized with a Zeta-Sizer Nano S (Malvern). The obtained correlograms were analyzed and converted to particle size distributions with the Malvern Panalytical software.

### Example 20—Statistical Analysis

The statistical analyses were performed using Prism v.8 software (GraphPad). Data sets with two samples were compared by applying a Student's t test to calculate two-tailed p-values.

TABLE 1
Sequences

| SEQ. | | | | |
|---|---|---|---|---|
| ID | | Common | Gene or | |
| No. | Species | Name | Protein | Sequence |
| 1 | N/A | | Reflectin | MEPM(X)₂M(X)MDF(X)₅DS(X)₁₀ |
| | | | N-terminal | |
| | | | Motif | |
| 2 | N/A | | Reflectin | PER(X)₂DM(X)₄MD(X)₂G(X)₁₁P |
| | | | Internal | |
| | | | Motif | |
| 3 | N/A | | Reflectin | (X)D(X)₅MD(X)₅M(X)6 |
| | | | C-terminal | |
| | | | Motif | |
| 4 | Doryteuthis | Longfin | Reflectin A1 | MAHHHHHHNRYLNRQRLYNMYRNKYR |
| | pealeii | inshore | N-His | GVMEPMSRMTMDFQGRYMDSQGRMVD |
| | | squid | | PRYYDYYGRMHDHDRYYGRSMFNQGH |
| | | | | SMDSQRYGGWMDNPERYMDMSGYQM |
| | | | | DMQGRWMDAQGRFNNPFGQMWHGRQ |
| | | | | GHYPGYMSSHSMYGRNMYNPYHSHYAS |
| | | | | RHFDSPERWMDMSGYQMDMQGRWMD |
| | | | | NYGRYVNPFNHHMYGRNMCYPYGNHY |
| | | | | YNRHMEHPERYMDMSGYQMDMQGRW |
| | | | | MDTHGRHCNPFGQMWHNRHGYYPGHP |
| | | | | HGRNMFQPERWMDMSGYQMDMQGRW |
| | | | | MDNYGRYVNPFSHNYGRHMNYPGGHY |
| | | | | NYHHGRYMNHPERHMDMSSYQMDMH |
| | | | | GRWMDNQGRYIDNFDRNYYDYHMY |
| 5 | Doryteuthis | Longfin | Reflectin-like | MNRYLNRQRLYNMYRNKYRGVMEPM |
| | pealeii | inshore | protein A1 | SRMTMDFQGRYMDSQGRMVDPRYYDY |
| | | squid | | YGRMHDHDRYYGRSMFNQGHSMDSQR |
| | | | | YGGWMDNPERYMDMSGYQMDMQGR |
| | | | | WMDAQGRFNNPFGQMWHGRQGHYPG |
| | | | | YMSSHSMYGRNMYNPYHSHYASRHFD |
| | | | | SPERWMDMSGYQMDMQGRWMDNYG |
| | | | | RYVNPFNHHMYGRNMCYPYGNHYYNR |
| | | | | HMEHPERYMDMSGYQMDMQGRWMD |
| | | | | THGRHCNPFGQMWHNRHGYYPGHPHG |
| | | | | RNMFQPERWMDMSGYQMDMQGRWM |
| | | | | DNYGRYVNPFSHNYGRHMNYPGGHYN |
| | | | | YHHGRYMNHPERHMDMSSYQMDMHG |
| | | | | RWMDNQGRYIDNFDRNYYDYHMY |
| 6 | Doryteuthis | Longfin | Reflectin-like | MNRYMMRHRPMYSNMYRTGRKYRGV |
| | pealeii | inshore | protein A2 | MEPMSRMTMDFQGRYMDSQGRMVDP |
| | | squid | | RYYDYGRCHDYDRYYGRSMFNYGPNM |
| | | | | DGQRYGGWMDFPERYMDMSGYQMDM |
| | | | | HGRWMDSQGRYCNPMGHSWSNRQGY |
| | | | | YPGSNYGRNMFNPERYMDMSGYQMD |
| | | | | MQGRWMDMGGRHVNPFSHSMYGRNM |
| | | | | FNPSYFSNRHMDNPERYMDMSGYQMD |
| | | | | MQGRWMDTQGRYMDPSWSNMYDNYN |
| | | | | SWY |
| 7 | Doryteuthis | Longfin | Reflectin-like | MSSFMDPMHYDGMGMSHSKTGDFSHN |
| | pealeii | inshore | protein B1 | CMRSFHKSQRDVMRRDIMGKSSKNRRF |
| | | squid | | GNLMEPMSRMTMDFHGRLIDSQGRIVD |
| | | | | PGHYFAMDDHYMENDRFLYPHDMLRN |
| | | | | RHGMYGFMQGDYGNNMHRGMFADGM |
| | | | | YRDMHHSGMNPSGYMHGGSMQNRPM |
| | | | | MYMQGRYLDDSYFMNYHDPPVIVHSH |
| | | | | YNDQEGRHHGMYDRHSDSYGSHRRHG |
| | | | | DSHSMPRRPSESHSPQRRPSEGHIIQVRP |
| | | | | EGGSSRKTSRAQLFPDDKLTDSA |
| 8 | Doryteuthis | California | Reflectin-like | MNRYLNRQRLYNMYRNKYRGVMEPM |
| | opalescens | market | protein Al | SRMTMDFQGRYMDSQGRMVDPRYYDH |
| | | squid | | YGRMHDYDRYYGRSMFNQGHSMDSQR |
| | | | | YGGWMDNPERYMDMSGYQMDMQGR |
| | | | | WMDAQGRYNNPFSQMWHSRQGHYPG |
| | | | | YMSHHSMYGRNMHYPYHSHSASRHFD |
| | | | | SPERWMDMSGYQMDMQGRWMDNYG |
| | | | | RYVNPFHHHMYGRNMFYPYGSHCNNR |
| | | | | HMEHPERYMDMSGYQMDMQGRWMD |
| | | | | THGRHCNPLGQMWHNRHGYYPGHPHG |
| | | | | RNMFQPERWMDMSSYQMDMQGRWM |
| | | | | DNYGRYVNPFSHNYGRHMNYPGGHYN |
| | | | | YHHGRYMNHPERQMDMSGYQMDMHG |
| | | | | RWMDNQGRYIDNFDRNYYDYHMY |
| 9 | Doryteuthis | California | Reflectin-like | MNRYMMRHRPMYSNMYRTGRKYRGV |
| | opalescens | market | protein A2 | MEPMSRMTMDFQGRYMDSQGRMVDP |
| | | squid | | RYYEYGRCHDYDRYNGRSMFNNGPYM |
| | | | | DGQRYGGWMDFPERYMDMSGYQMDM |
| | | | | HGRWMDSQGRYCNPMGHSWSNRQGY |
| | | | | YPGSNYGRNMFNPERYMDMSGYQMD |
| | | | | MQGRWMDMGGRHVNPFSHSMYGRNM |
| | | | | FNPSYFSNRHMDNPERYMDMSGYQMD |
| | | | | MQGRWMDTQGRYMDPSMSNMYDNYN |
| | | | | YWY |
| 10 | Doryteuthis | California | Reflectin-like | MSSFMDPMHYDGMGMSHSKSGDFSHN |
| | opalescens | market | protein B1 | CMRSFHKSQRDGMRRDIMGKSSKNRRF |
| | | squid | | GNLMEPMSRMTMDFHGRLIDSQGRIVD |
| | | | | RHGMYGFMQGDYGNNMHRGMFADGM |
| | | | | YRDMHHSGMNPSSYMHGGS |
| 11 | Doryteuthis | California | Reflectin-like | MQNRPMMYMQGRYLDDSYFMNYHDP |
| | opalescens | market | protein C1 | PVIVHSHYNDQEGRHQGMYDRHSDSYG |
| | | squid | | SHRRHGDSHSMPRRPSESHSPQRRPSEG |
| | | | | HIIQVRPEGGSSRKTSRAQLFPDDKLTDS |
| | | | | A |
| 12 | Loligo | Veined | Methionine- | MNRSMNRYQPSNMWGNMNRDRYSGM |
| | forbesii | squid | rich repeat | MEPMSRMSMDFQGRHMDSMDRMVDP |
| | | | protein 1 | GRWNDYDRYYGRSTFNYGWMENGDRF |
| | | | | NRNLRPMDFPERYMDMSDYQMDMGG |
| | | | | RWMDPYGRQCNPFNQCGYNRHGYYPG |
| | | | | YSYGRNMCYPERWMDMSNYSMDMQG |
| | | | | RYMDRRGRHCNPFSQHTNWYGRYRNY |
| | | | | PGDNNYYNRNMYYPERHFDMSNWQM |
| | | | | DMQGRWMDNQGRYNNPYWYGRNMY |
| | | | | QPYQNNQWSGRWDYPGMDCGMDMQG |
| | | | | GYMNNSNEGDYL |
| 13 | Sepia | Common | Reflectin | MNRSMNRWRPMFNNMHNNYYGRSMF |
| | officinalis | cuttlefish | | NYDWMMDGDRYNRYYRWMDFPERY |
| | | | | MDMSGYQMDMYGRWMDMQGQHYNP |
| | | | | FRQWGYQRHGYYPGYHYGSNMFYPER |
| | | | | WMDMSNYSMDMQGRYMDRWGRHCN |
| | | | | PFSHYYNHWNRYWNHPGYYNNHYNRH |
| | | | | MYYPERYFDMSNWQMDMQGRWMDM |
| | | | | QGRHCNPYWYNWHGRHMYYPYQNYY |
| | | | | WYGRWDYPGMDYSNWQMDFQGRWM |
| | | | | DNHGRHMNPWWNEHYFNHYY |
| 14 | Sepia | Common | Reflectin 4 | MNRSMNRWRPMFNNMHNNYYGRSMF |
| | officinalis | cuttlefish | | NYDWMMDGDRYNRYYRWMDFPERY |
| | | | | MDMSGYQMDMYGRWMDMQGQHYNP |
| | | | | FRQWGYQRHGYYPGYHYGSNMFYPER |
| | | | | WMDMSNYSMDMQGRYMDRWGRHCN |
| | | | | PFSHYYNHWNRYWNHPGYYNNHYNRH |
| | | | | MYYPERYFDMSNWQMDMQGRWMDM |
| | | | | QGRHCNPYWYNWHGRHMYYPYQNYY |
| | | | | WYGRWDYPGMDYSNWQMDFQGRWM |
| | | | | DNHGRHMDPWWNEQYFNHYY |
| 15 | Sepia | Common | Reflectin 2 | MNRFMNRYRPMFNNMHNNYYGRSMF |
| | officinalis | cuttlefish | | NYDWMMDGDRYNRYYRWMDFPERY |
| | | | | MDMSGYQMDMYGRWMDMQGHHCNP |
| | | | | FRQWGYQRHGYYPGFHYGSNMFYPER |
| | | | | WMDMSNYSMDMQGRYMDRWGRHCN |
| | | | | PFSHYYNHWNRYWNHPGYYNYHYNRH |
| | | | | MYYPERYFDMSNWQMDMQGRWMDM |
| | | | | QGRHSNPYWYNWHGRHMYYPYQNYY |
| | | | | WYGRWDHHGMDYSNWQMDFQGRWM |
| | | | | DNHGRHMNPWWNEQYFNHYY |
| 16 | Sepia | Common | Reflectin 3 | MNRFMNRYRPMFNNMHNNYYGRSMF |
| | officinalis | cuttlefish | | NYDWMMDGDRYNRYYRWMDFPERY |
| | | | | MDMSGYQMDMYGRWMDMQGRHCNP |
| | | | | FRQWGYQRHGYYPGFHYGSNMFYPER |
| | | | | WMDMSNYSMDMQGRYMDRWGRHCN |
| | | | | PFSQYYNHWNRYWNHPGYYNNHYNRH |
| | | | | MYYPERYFDMSNWQMDMQGRWMDM |
| | | | | QGRHSNPYWYNWHGRHMYYPYQNYY |
| | | | | WYGRWDHHGMDYSNWQMDFQGRWM |
| | | | | DNHGRHMDPWWNEQYFNHYY |
| 17 | Sepia | Common | Reflectin 5 | MNRFMNRYRPMFNNMHNNYYGRSMF |
| | officinalis | cuttlefish | | NYDWMMDGDRYNRYNRWMDFPERY |
| | | | | MDMSGYQMDMYGRWMDMQRHHCNP |
| | | | | FRQWGYQRHGYYPGFHYGSNMFYPER |
| | | | | WMDMSNYSMDMQGRYMDRWGRHCN |
| | | | | PFSHYYNHWNRYWNHPGYYNNHYNRH |
| | | | | MYYPERYFDMSNWQMDMQGRWMDM |
| | | | | QGRHSNPYWYNWHGRHMYYPYQNYY |
| | | | | WYGRWDHHGMDYSNWQMDFQGRWM |
| | | | | DNHGHHMNPWWNEQYFNHYY |
| 18 | Sepia | Common | Reflectin | MNRSMNRWRPMFNNMHNNYYGRSMF |
| | officinalis | cuttlefish | | NYNWMMDGDRYNRYYRWMDFPEWY |
| | | | | MDMSGYQMDMYGRWMDMHGRHCNP |
| | | | | FRQWGYQRHGYYPGFHYGSNMFYPER |
| | | | | WMDMSNYSMDMQGRYMDRWGRHCN |
| | | | | PFSHYYNHWNRYWNHPGYYNYYYMY |
| | | | | YPERCYDMSNWQMDMQGRWMDMQG |
| | | | | RHSNPYWYNWHGRHMYYPYQNYYWY |
| | | | | GRWDHHGMDYSNWQMDFQGRWMDN |
| | | | | HGRHMDPWWNEHYFNHYY |
| 19 | Sepia | Common | Reflectin 7 | MNRFMNRYRPMFNNMHNNYYGRSMF |
| | officinalis | cuttlefish | | NYNWMMDGDRYNRYYRWMDFPERY |
| | | | | MDMSGYQMDMYGRWMDMQGRHCNP |
| | | | | FRQWGYQRHGYYPGFHYGSNMFYPER |
| | | | | WMDMSNYSMDMQGRYMDRWGRHCN |
| | | | | PFSYYYNHWNRYWNHPGYYNYYYMY |
| | | | | YPERYYDMSNWQMDMQGRWMDMQG |
| | | | | RHSNPYWYNWHGRHMYYPYQNYYWY |
| | | | | GRWDYPGMDYSNWQMDFQGRWMDN |
| | | | | HGRHMNPWWNEQYFNHYY |
| 20 | Sepia | Common | Reflectin 6 | MNRSMNRWRPMFNNMHNNYYGRSMF |
| | officinalis | cuttlefish | | NYNWMMDGDRYNRYYRWMDFPEWY |
| | | | | MDMSGYQMDMYGRWMDMHGRHCNP |
| | | | | FRQWGYQRHGYYPGFHYGSNMFYPER |
| | | | | WMDMSNYSMDMQGRYMDRWGRHCN |
| | | | | PFSHYYNHWNRYWNHPGYYNYYYMY |
| | | | | YPERYYDMSNWQMDMQGRWMDMQG |
| | | | | RHSNPYWYNWHGRHMYYPYQNYYWY |
| | | | | GRWDHHGMDYSNWQMDFQGRWMDN |
| | | | | HGRHMDPWWNEHYFNHYY |
| 21 | Sepia | Common | Reflectin | MNRFMNRWRPMFNNMRNNMYRGRYR |
| | officinalis | cuttlefish | | GMMEPMSRMTMDFQGRYMDSQGRMV |
| | | | | DPRYYDYYGRWNDYDRYYGRSMFNYS |
| | | | | WMMDGDRYNRYYRWMDFPERYMDM |
| | | | | SGYQMDMYGRWMDMHGRHCNPFRQW |
| | | | | GYQRHGYYPGFHYGSNMFYPERWMDM |
| | | | | SHYSMDMQGRYMDRWGRHCNPFSHYY |
| | | | | NHWNRYWNHPGYYNNHYNRHMYYPE |
| | | | | RYFDMSNWQMDMQGRWMDMQGRHS |
| | | | | DPYWYNWHGRHMYYPYQNYYWYGR |
| | | | | WDHHGMDYSNWQMDFQGRWMDNHG |
| | | | | RHMDPWWNEHYFNHYY |
| 22 | Sepia | Common | Reflectin 10 | MNRFMNRWRPMFNNMRNNMYRGRYR |
| | officinalis | cuttlefish | | GMMEPMSRMTMDFQGRYMDSQGRMV |
| | | | | DPRYYDYYGRWNDYDRYYGRSMFNYS |
| | | | | WMMDGDRYNRYYRWMDFPERYMDM |
| | | | | SGYQMDMYGRWMDMHGRHCNPFRQW |
| | | | | GYQRHGYYPGFHYGSNMFYPERWMDM |
| | | | | SHYSMDMQGRYMDRWGRHCNPFSHYY |
| | | | | NHWNRYWNHPGYYNNHYNRHMYYPE |
| | | | | RYFDMSNWQMDMQGRWMDMQGRHS |
| | | | | NPYWYNWHGRHMYYPYQNYYWYGR |
| | | | | WDHHGMDYSNWQMDFQGRWMDNHG |
| | | | | RHMDPWWNEHYFNHYY |
| 23 | Sepia | Common | Reflectin 11 | MNRFMNRWRPMFNNMHNNMYRGRYR |
| | officinalis | cuttlefish | | GMMEPMSRMTMDFQGRYMDSQGRMV |
| | | | | DPRYYDYYGRWNDYDRYYGRPMFNYS |
| | | | | WMMDGDRYNRNYRWMDFPERYMDM |
| | | | | SGYQMDMYGRWMDMQGHHCNPFRQ |
| | | | | WGYQRHGYYPGFHYGSNMFYPERWM |
| | | | | DMSNYSMDMQGRYMDRWGRHCNPFS |
| | | | | HYYNHWNRYWNHPGYYNNHYNRHMY |
| | | | | YPERYFDMSNWQMDMQGRWMDMQG |
| | | | | RHSNPYWYNWHGRHMYYPYQNYYWY |
| | | | | GRWDHPGMDYSNWQMDFQGRWMDN |
| | | | | HGRHMNPWWNEHYFNHYY |
| 24 | Sepia | Common | Reflectin | MNRYMNRFRNWYGNNYRGRYRGMME |
| | officinalis | cuttlefish | | PMSRMTMDFQGRYMDSQGRMVDPRYY |
| | | | | DYYGRWNDYDRYYGRPMFNYSWMMD |
| | | | | GDRYNRYYRWMDFPERYMDMSGYQM |
| | | | | DMYGRWMDMQGRHCNPFRQWGYQRH |
| | | | | GYYPGYHYGSNMFYPERWMDMSNYS |
| | | | | MDMQGRYMDRWGRHCNPFSHYYNHW |
| | | | | NRYWNHPGYYNYYYMYYPERYYDMS |
| | | | | NWQMDMQGRWMDMQGRHCNPYWYN |
| | | | | WHGRHMYYPYQNYYWYGRWDYPGM |
| | | | | DYSNWQMDFQGRWMDNQGRYMDPW |
| | | | | WWNDYYSYYY |
| 25 | Sepia | Common | Reflectin 9 | MNRYMNRFRNWYGNNYRGRYRGMME |
| | officinalis | cuttlefish | | PMSRMTMDFQGRYMDSQGRMVDPRYY |
| | | | | DYYGRWNDYDRYYGRSMFNYSWMMD |
| | | | | GDRYNRYYRWMDFPERYMDMSGYQM |
| | | | | DMYGRWMDMQGRHCNPFRQWWYHR |
| | | | | HGYYPGFHYGSNMFYPERWMDMSNYS |
| | | | | MDMQGRYMDRWGRHCNPFSHYYNHW |
| | | | | NRYWNHPGYYNYYYMYYPERYYDMS |
| | | | | NWQMDMQGRWMDMQGRHSNPYWYN |
| | | | | WHGRHMYYPYQNYYWYGRWDYPGM |
| | | | | DYSNWQMDFQGRWMDNQGRYMDPW |
| | | | | WWNDYYYNYYY |
| 26 | Sepia | Common | Reflectin | MNRYTMRNRPMYGNMYRTGKKYRGV |
| | officinalis | cuttlefish | | MEPMSRMTMDFQGRYMDSQGRMVDP |
| | | | | RHNDYYGRWNDYDRYYGRSMFNYGPH |
| | | | | MDGHQHGGWMDFPERWMDMSNYQM |
| | | | | DMQGRWMDMQGRHCQPFNQWGYNRH |
| | | | | GNYPSSYYGRNMFYPERWMDMSNWQ |
| | | | | MDTQGRWMDMQGRYGSPFNQWGYNR |
| | | | | HGYYPGSSYGRNMYHPERWMDMSNYQ |
| | | | | MDMQGRWMDMHGRHVNPFSHSMHGR |
| | | | | NWSYPYYNYYSSRHMDYPERNMDMSN |
| | | | | WQMDMQGRWMDMQGRHMDPSWSNM |
| | | | | HDNHNYWF |
| 27 | Sepia | Common | Reflectin 1 | MNRYMMRNRPMYGNMYRTGKKYRGV |
| | officinalis | cuttlefish | | MEPMSRMTMDFQGRYMDSQGRMVDP |
| | | | | RHNDYYGRWNDYDRYYGRSMFNYGPH |
| | | | | MDGHQHGGWMDFPERWMDMSNYQM |
| | | | | DMQGRWMDMQGRHCQPFNQWGYNRH |
| | | | | GNYPSSYYGRNMFYPERWMDMSNWQ |
| | | | | MDTQGRWMDMQGRYGSPFNQWGYNR |
| | | | | HGYYPGSSYGRNMYHPERWMDMSNYQ |
| | | | | MDMQGRWMDMHGRHVNPFSHSMHGR |
| | | | | NWSYPYYNYYSSRHMDYPERNMDMSN |
| | | | | WQMDMQGRWMDMQGRHMDPSWSNM |
| | | | | HDNHNYWF |
| 28 | Sepia | Common | Reflectin 8 | MNRFMNRYRPMFNNMHNNMYNNMYR |
| | officinalis | cuttlefish | | GRYRGMMEPMSRMTMDFQGRYMDSQ |
| | | | | GRMVDPRYYDYYGRWNDYDRYYGKS |
| | | | | MFNYGWMMDGDRYNNYYRWMDFPER |
| | | | | YMDMSGYQMDMYGRWMDMQGRHCN |
| | | | | PFNQWGHNRYGQSFNYNYGRNMFYPE |
| | | | | RWMDMSNYSMDMQGRYMDRWGRHC |
| | | | | NPFSQNMNWYGRYWNYPGYNNYYYN |
| | | | | RHMYYPERYFDMSNWQMDMQGRWM |
| | | | | DMQGRHNNPYWYNWYGRQMYYPYQN |
| | | | | NWYGRWDYPGMDYSNWQMDMQGRW |
| | | | | MDMQGRYMDPWMSDYSYNN |
| 29 | Sepia | Common | Reflectin_like | MSTFMDPMFYEGLGMPPPNFGDFNHNC |
| | officinalis | cuttlefish | | MRSFHKSQRDMMRRDIMAKSSKNKRC |
| | | | | GDLMEPMSRMIMDFNGGLIDSRRRITDS |
| | | | | DHYFTIDGNYGDNEKPLTSDGLLRNRY |
| | | | | DMYAFAPADKCHNRTRGLYGDSMYRD |
| | | | | KHQDGMYSSGYMQGRSMQNHRMMGS |
| | | | | FQGGMQTQSRYMDDPYYVNYNSGTYD |
| | | | | TPVDMNSYYFDQEGRHRMYSRFSEGQI |
| | | | | TPGRQEGTYSARRESRSGQRRLSDSHSF |
| | | | | QRPIDTRSNRRTSHGMLYSERNNIDFA |
| 30 | Euprymna | Hawaiian | Reflectin 1a | MNRFMNRYRPMFNNMYSNMYRGRYR |
| | scolopes | bobtail | | GMMEPMSRMTMDFQGRYMDSQGRMV |
| | | squid | | DPRYYDYYGRFNDYDRYYGRSMFNYG |
| | | | | WMMDGDRYNRYNRWMDYPERYMDM |
| | | | | SGYQMDMSGRWMDMQGRHCNPYSQW |
| | | | | MMYNYNRHGYYPNYSYGRHMFYPER |
| | | | | WMDMSNYSMDMYGRYMDRWGRYCN |
| | | | | PFSQYMNYYGRYWNYPGYNNYYYSRN |
| | | | | MYYPERYFDMSNWQMDMQGRWMDN |
| | | | | QGRYCSPYWNNWYGRHMYYPYQNNY |
| | | | | FYGRYDYPGMDYSNYQMDMQGRYMD |
| | | | | QYGMNDYYY |
| 31 | Euprymna | Hawaiian | Reflectin 1b | MNRFMNKYRPMFNNMYSNMYRGRNR |
| | scolopes | bobtail | | GMMEPMSRMTMDFQGRYMDSQGRMV |
| | | squid | | DPRYYDYYGRFNDYDRYYGRSMFNYG |
| | | | | WMMDGDRYNRYNRWMDYPERYMDM |
| | | | | SGYQMDMSGRWMDMQGRHCNPYSQW |
| | | | | GYNYNRHGYYPNYSYGRHMFYPERWM |
| | | | | DMSGYQMDMQGRYMDRWGRYCNPFS |
| | | | | QYMNYYGRYWNYPGYNSYYNSRNMF |
| | | | | YPERYFDMSNWQMDMQGRWMDNQGR |
| | | | | YCSPYWNNWYGRQMYYPYQNNYFYG |
| | | | | RYDYPGMDYSNYQMDMQGRYMDQYG |
| | | | | MNDYCY |
| 32 | Euprymna | Hawaiian | Reflectin 2a | MNRYMTRFRNFYGNMYRGRYRGMME |
| | scolopes | bobtail | | PMSRMTMDFQGRYMDSQGRMVDPRYY |
| | | squid | | DYYGRYNDYDRYYGRSMFNYGWMMD |
| | | | | GDRYNRYNRWMDFPERYMDMSGYQM |
| | | | | DMYGRWMDMQGRHCNPYSQWMMYN |
| | | | | YNRHGYYPNYSYGRHMFYPERWMDMS |
| | | | | NYSMDMYGRYMDRWGRYCNPFYQFY |
| | | | | NHWNRYGNYPGYYNYYYMYYPERYFD |
| | | | | MSNWQMDMQGRWMDMQGRYCSPYW |
| | | | | YNWYGRHMYYPYQNYYWYGRYDYPG |
| | | | | MDYSNWQMDMQGRWMDMQGRYMD |
| | | | | YPYNYYNWY |
| 33 | Euprymna | Hawaiian | Reflectin 2b | MNRYMNRFRNFYGNMYRGRYRGMME |
| | scolopes | bobtail | | PMSRMTMDFQGRYMDSQGRMVDPRFY |
| | | squid | | DYYGRYNDYDRYYGRSMFNYGWMMD |
| | | | | GDRYNRCNRWMDYPERYMDMSGYQM |
| | | | | DMYGRWMDMQGRHCNPYSQWMMYN |
| | | | | YNRHGYYPNYSYGRHMFYPERWMDMS |
| | | | | NYSMDMYGRYMDRWGRYCNPFYQFY |
| | | | | NHWNRYGNYPGYYNYYYMYYPERYFG |
| | | | | MSNWQMDMQGRWMDMQGRYCSPYW |
| | | | | YNWYGRHMYYPYQNYYWYGRYDYPG |
| | | | | MDYSNWQMDMQGRWMDMQGRYMD |
| | | | | YPYNYYNWNH |
| 34 | Euprymna | Hawaiian | Reflectin 2c | MNRYMNRFRNFYGNMYRGRYRGMME |
| | scolopes | bobtail | | PMSRMTMDFQGRYMDSQGRMVDPRYY |
| | | squid | | DYYGRYNDYDRYYGRSMFNYGWMMD |
| | | | | GDRYNRYNRWMDFPERYMDMSGYQM |
| | | | | DMYGRWMDMQGRHCNPYSQWMMYN |
| | | | | YNRHGYYPNYSYGRHMFYPERWMDMS |
| | | | | NYSMDMYGRYMDRWGRYCNPFYQFY |
| | | | | NHWNRYGNYPGYYNYYYMYYPERYFD |
| | | | | MSNWQMDMQGRWMDMQGRYCSPYW |
| | | | | YNWYGRQMYYPYQNYYWYGRYDYPG |
| | | | | MDYSNYQMDMQGRYMDMQGRYMDY |
| | | | | PYNYYNWNH |
| 35 | Euprymna | Hawaiian | Reflectin 2d | MNRYMNRFRNFYGNMYRGRYRGMME |
| | scolopes | bobtail | | PMSRMTMDFQGRYMDSQGRMVDPRYY |
| | | squid | | DYYGRFNDYDRYYGRSMFNYGWMMD |
| | | | | GDRYNRYNRWMDFPERYMDMSGYQM |
| | | | | DMYGRWMDMQGRHCNPYSQWMMYN |
| | | | | YNRHGYYPNYSYGRHMFYPERWMDMS |
| | | | | NYSMDMYGRYMDRWGRYCNPFYQFY |
| | | | | NHWNRYGNYPGYYSYYYMYYPERYFD |
| | | | | MSNWQMDMQGRWMDMQGRYCSPYW |
| | | | | YNWYGRHMYYPYQNYYWYGRYDYPG |
| | | | | MDYSNWQMDMQGRWMDMQGRYMD |
| | | | | YPYNYYNWNH |
| 36 | Euprymna | Hawaiian | Reflectin 3a | MNRYMNRFRNFYGNMCRNRNRGMME |
| | scolopes | bobtail | | PMSRMTMDFQGRYMDSQGRMVDPRYY |
| | | squid | | DYYGRYNDYDRYYGRSMFNYGWMMD |
| | | | | GDRYNRYNRWMDYPERYMDMSGYQM |
| | | | | DMYGRWMDMQGRHCNPYSQWMMYN |
| | | | | YNRHGYYPNYSYGRHMFYPERWMDMS |
| | | | | NYSMDMYGRYMDRWGRYCNPFYHYY |
| | | | | NHWNRSGNNPGYYSYYYMYYPERYFD |
| | | | | MSNWQMDMQGRWMDMQGRYCSPYW |
| | | | | YNWYGRQMYYPYQNYYWYGRWDYPG |
| | | | | MDYSNWQMDMQGRWMDMQGRYMD? |
| | | | | WWMNDSYYNNYYN |
| 37 | Euprymna | Hawaiian | Uncharacterized | MYYPERYFDMSNWQMDMQGRWMDM |
| | scolopes | bobtail | protein | QGRYCSPYWYNWYGRQMYYPYQNYY |
| | | squid | | WYGRWDYPGMDYSNWQMDMQGRWM |
| | | | | DMQGRYMDPWWMNDSYYNNYYN |
| 38 | Octopus | California | Uncharacterized | MNRSRNMFRNSSRKHRGVMEPMTRMT |
| | bimaculoides | two-spotted | protein | MDFQGRYLDSSGRLVEPRCNDYYGRNS |
| | | octopus | | NYDRYRPMQNTGIYDNDKFQKYGRFM |
| | | | | HFPERQMDMSGYQMDMRGRYMDKYG |
| | | | | RHCNPYSRRHMNYPNNNYDNYHMYNP |
| | | | | EKLMDMSNFQMDMHGRWMDSNGRYS |
| | | | | SPFSNYGSRHHQNYPHFNYNWGQRGFN |
| | | | | YPDRFFDMSNYQMDLDGKWMDTYGR |
| | | | | HCHPFYDNSNYYGKQYNYNMYPHYNY |
| | | | | NWGQKYYHYPERYFDMSNYQMDFDGR |
| | | | | WMDMFGRSHSPFNGYNNNQGRQHHGQ |
| | | | | PHNSFSYGQRYQDRNFDIGNYQMDFDG |
| | | | | RWMDMYGRYSHPFYGYNNFQSRYQHN |
| | | | | LPQNFNWGQRSFHNPERLFDMGNYQM |
| | | | | DFDGHWMDMDDRHCQPFTGNYNHSNR |
| | | | | YQQNCNHSPSQNFNWSQRYQDNPEKFF |
| | | | | DMSGYQMEFDGRWMDSNNYNSDNFW |
| 39 | Octopus | California | Uncharacterized | MYGHIYNGRMEPMHMMSMDFHGRYM |
| | bimaculoides | two-spotted | protein | DSYGRMVDPRSYGFYGRYHDQDSYYG |
| | | octopus | | RSMYNNHNFHDFDRFHRFDYFMNFPD |
| | | | | MFMDMSGYEMDFNGRWMNMHRF |
| 40 | Octopus | California | Uncharacterized | HPFHSFSGYHGYHHSGYYPYHSYSQGR |
| | bimaculoides | two-spotted | protein | RYHNYYDMFYDMSHYQMDFDGNWMD |
| | | octopus | | MYNHYSHPFFGYNHYHRGSHYYNHYP |
| | | | | YHNYSWGHRFYDYPERFFDMSHYEMD |
| | | | | FNGRWMNMHRF |
| 41 | Octopus | California | Uncharacterized | MFYDMSHYQMDFDGNWMDMYNHYSH |
| | bimaculoides | two-spotted | protein | PFFGYDHYHRGSHYYNHYPYHNYSWG |
| | | octopus | | HRFYDYPERFFDMSHYEMDFNGRWMN |
| | | | | MHRF |
| 42 | Octopus | California | Uncharacterized | MNRLMNKFRHHFGRKYRGIMEPMSVM |
| | bimaculoides | two-spotted | protein | SMDFQGRYMDSYGRMVDPRFYEFYGR |
| | | octopus | | YSDNDRYYGKSMYNYYGFYDNDRFHR |
| | | | | YGNFMDFPERFMDMSSYQMDMYGRW |
| | | | | MDMHGHHSSPYWYMFNSSRHGHYPGY |
| | | | | RYGRNWFYPERFMDMSHYQMDMYGR |
| | | | | YMDRYGRQCNPYYNYYRRYMYYPYM |
| | | | | NFYYMHYPERFMDMSGYQMDMYGRW |
| | | | | MDMYGRHSTPFYTNYGRYYHNYPYYN |
| | | | | YSWGQRYYNYPERYFDMTNYQMDFDG |
| | | | | RWMDMYSRHCTPFYSYHGRYHHYYPY |
| | | | | HSYSWGQRFYNNPERWYDMDYEFHSM |
| | | | | SPYNYHSRYHYFNYSPYFSGGHRWFDM |
| | | | | SNYQMDFGGQWMDMNGRYMNHFDH |
| | | | | WNEYFF |
| 43 | Octopus | California | Uncharacterized | MNRYMNRRNNFSRRYRGIMEPMSRMT |
| | bimaculoides | two-spotted | protein | MDFQGRYMDSYGRMVDPRFYGFYGRY |
| | | octopus | | SDNDRYYGRSMYNYYGFYDNDRFHRY |
| | | | | GNFMDFPERFMDMSGYQMDMSGRWM |
| | | | | DMHGHYSSPHWHMFNSSRQGYYPGYH |
| | | | | YGRNWFYPERFMDMSHYQMDMYGRY |
| | | | | MDRNGRHCNPYYNYYRRYMYYPYMN |
| | | | | FYHMYYPERFMDMSGYQMDMYGRWM |
| | | | | DMSGRHSSPFYSYHSRFHHNYPYYNYS |
| | | | | WGQRYYNYPERYFDMGNYQMDFDGR |
| | | | | WMDMYSRHCTPFYNYHGKFHHNYPYY |
| | | | | NYSWGQRYYNYPERYFDMGNYQMDFD |
| | | | | GRWMDNYGRYSSPFNSYHGRFHHNYP |
| | | | | YYNYSWGQRYYNYPERYFDMGNYQM |
| | | | | DFDGRWMDNYGRYSSPFYNYHGRYHN |
| | | | | YPYYNYSWGQRYYNYPEGNYQMDFDG |
| | | | | RWMDNYGRYFHGYNYHNRHYYNSYP |
| | | | | NSYNYNWGQRYYDYPERNFDMFNYQM |
| | | | | DFDSRWMDGQNFHYYGDNYNY |
| 44 | Octopus | California | Uncharacterized | MNRFMNRFRPQFNRKYRGFMEPMNMM |
| | bimaculoides | two-spotted | protein | SMVFQGRYMDSYGKMVDPKLYEFYGK |
| | | octopus | | YSDNDRYYGKSMYNYYGFYDNDRFHR |
| | | | | NGNFMDFPERFMDMSGYQMDMNGKW |
| | | | | MDTQGQNSHPYWNMFSSSRQGCYPGY |
| | | | | SYGRNWFFPERFMDMSHYQMDMNGRY |
| | | | | MDKSGRHCNPYYSYYRRYMSHPQMNF |
| | | | | NQMHYPERFMDMSSYQMDIGGRYMDK |
| | | | | WGCHINPFSTYYFGKQSYFPHNYWSQR |
| | | | | KYMDMSSYQMDMQYNSMDMNSRNCD |
| | | | | QLHYFRNFDMWNNQMDFDGHWMNM |
| | | | | NNQSYHPSSFIRNQVYYNPYHFYTWMS |
| | | | | RYYNHPEKFYDTSNYQVEFGGKWPSQE |
| | | | | YECITQE |

### DOCTRINE OF EQUIVALENTS

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

1. A living biological cell comprising:
a first cellular component characterized by a first non-native reflectin biomolecule.

2. The living biological cell of claim 1, wherein the first reflectin biomolecule is selected from the group consisting of: a natural isoform of reflectin protein, a non-natural variant of reflectin protein, a non-natural biomolecule derived from or mimicking reflectin protein, and any combination thereof.

3. The living biological cell of claim 1, wherein the first cellular component is characterized by a tunable refractive index due to the first non-native reflectin biomolecule.

4. The living biological cell of claim 1, wherein the first cellular component is characterized by a tunable optical property due to the first non-native reflectin biomolecule.

5. The living biological cell of claim 1, further comprising a second cellular component is characterized by a second non-native reflectin biomolecule.

6. The living biological cell of claim 5, wherein the second reflectin biomolecule is selected from the group consisting of: a natural isoform of reflectin protein, a non-natural variant of reflectin protein, a non-natural biomolecule derived from or mimicking reflectin protein, and any combination thereof.

7. The living biological cell of claim 5, wherein the second cellular component is characterized by a tunable refractive index due to the second non-native reflectin biomolecule.

8. The living biological cell of claim 5, wherein the second cellular component is characterized by a tunable optical property due to the second non-native reflectin biomolecule.

9. The living biological cell of claim 1, wherein the living biological cell is of a cell type selected from the group consisting of: a bacterial cell, an archaeal cell, a plant cell, an animal cell and a fungal cell.

10. The living biological cell of claim 1, wherein the living biological cell is a mammalian cell.

11. The living biological cell of claim 1 or 5, wherein the first cell component or second cell component is selected from the group consisting of: an organelle, a protein, a membrane, a cytoskeleton, and a ribosome.

12. The living biological cell of claim 4 or 8, wherein the tunable optical property is selected from the group consisting of: transmittance, reflectance, absorptance, and any combination thereof.

13. The living biological cell of claim 4 or 8, wherein the tunable optical property is selected from the group consisting of: transparency, opaqueness, coloration, iridescence, and any combination thereof.

14. The living biological cell of claim 1 or 5, wherein the first reflectin biomolecule or second reflectin biomolecule is the natural isoform of reflectin protein selected from the group consisting of: reflectin A1, reflectin A2, reflectin B1, reflectin C1, and another isoform or homologue; and any truncated or augmented version thereof.

15. The living biological cell of claim 1, wherein the living biological cell is a mammalian cell and the first reflectin biomolecule is reflectin A1.

16. The living biological cell of claim 3 or 7, wherein the tunable refractive index is tunable by application of an external stimulus selected from the group consisting of: ionic strength of the environment of the living biological cell, aromatic compounds, chemical, biological, biophysical, electrical, and mechanical stimulus, wherein the stimulus affects conformation and aggregation of reflectin biomolecule and or any of reflectin protein's isoforms, and any combination thereof.

17. The living biological cell of claim 3 or 7, wherein the tunable refractive index is tunable by application of NaCl or acetylcholine.

18. The living biological cell of claim 3 or 7, wherein the tunable optical property is tunable by application of an external stimulus selected from the group consisting of: ionic strength of the environment of the living biological cell, aromatic compounds, chemical, biological, biophysical, electrical, and mechanical stimulus, wherein the stimulus affects conformation and aggregation of reflectin biomolecule and or any of reflectin protein's isoforms, and any combination thereof.

19. The living biological cell of claim 4 or 8, wherein the tunable optical property is tunable by application of NaCl or acetylcholine.

20. The living biological cell of claim 1 or 5, wherein the first non-native reflectin biomolecule or second non-native reflectin biomolecule form a subcellular photonic architecture characterized by an architecture shape and an architecture size.

21. The living biological cell of claim 20, wherein the architecture shape is a shape selected from the group consisting of: spheroid, platelet, microfiber, hexagonal plate, film, and any other fundamental geometric shape.

22. The living biological cell of claim 20, wherein the architecture size is in a range from nanometers to tens of microns.

23. The living biological cell of claim 20, wherein the architectural size is in the range of approximately 5 nm to 5 μm.

24. The living biological cell of claim 20, wherein the architecture shape and the architecture size are tunable by application of an external stimulus selected from the group consisting of: ionic strength of the environment of the living biological cell, aromatic compounds, chemical, biological, biophysical, electrical, and mechanical stimulus, wherein the stimulus affects conformation and aggregation of reflectin biomolecule and or any of reflectin protein's isoforms, and any combination thereof.

25. The living biological cell of claim 20, wherein the architecture shape and the architecture size are tunable by application of NaCl or acetylcholine.

26. The living biological cell of claim 3 or 7, wherein the tunable refractive index is adjustable within the range of ˜1.40 to ˜1.62.

27. A living biological system comprising
a plurality of living biological cells, wherein each living biological cell in the plurality of living biological cells comprises a cellular component characterized by a non-native reflectin biomolecule.

28. The living biological system of claim 27, wherein the plurality of living biological cells are a cell type selected from the group consisting of: bacterial cells, archaeal cells, plant cells, animal cells, and fungal cells.

29. The living biological system of claim 27, wherein the plurality of living biological cells are mammalian cells.

30. The living biological system of claim 27, wherein the cellular component is characterized by a tunable refractive index due to the reflectin biomolecule.

31. The living biological system of claim 30, wherein the tunable refractive index is tunable by application of an external stimulus selected from the group consisting of: ionic strength of the environment of the living biological cell, aromatic compounds, chemical, biological, biophysical, electrical, and mechanical stimulus, wherein the stimulus affects conformation and aggregation of reflectin biomolecule and or any of reflectin protein's isoforms, and any combination thereof.

32. The living biological system of claim 30, wherein the tunable refractive index is tunable by application of NaCl or acetylcholine.

33. The living biological system of claim 30, wherein the tunable refractive index is adjustable within the range of ˜1.40 to ˜1.62.

34. The living biological system of claim 27, wherein the cellular component is characterized by a tunable optical property due to the reflectin biomolecule.

35. The living biological system of claim 34, wherein the tunable optical property is tunable by application of an external stimulus selected from the group consisting of: ionic strength of the environment of the living biological cell, aromatic compounds, chemical, biological, biophysical, electrical, and mechanical stimulus, wherein the stimulus affects conformation and aggregation of reflectin biomolecule and or any of reflectin protein's isoforms, and any combination thereof.

36. The living biological system of claim 34, wherein the tunable optical property is tunable by application of NaCl or acetylcholine.

37. The living biological system of claim 27, wherein the living biological system is a system selected from the group consisting of: an organ, a tissue, an organism.

38. A method of controlling a refractive index and optical properties of a living cell and subcellular components comprising:
providing a living biological cell, wherein the living biological cell comprises:
various cell components, and
a plurality of reflectin biomolecules, wherein the reflectin biomolecule is a biomolecule selected from the group consisting of: a natural isoform of reflectin protein, a non-natural variant of reflectin protein, a non-natural biomolecule derived from or mimicking reflectin protein, and any combination thereof, wherein
the living biological cell and the various cell components are each individually characterized by a tunable refractive index and tunable optical properties; and
applying to the living biological cell an external stimulus capable of affecting the conformation or aggregation of the reflectin biomolecule to affect the tunable refractive index and the tunable optical properties of the plurality of reflectin biomolecules and of their immediate surrounding.

39. A method of comprising
transfecting a living biological cell with a plasmid encoding for expression of a reflectin biomolecule, wherein the reflectin biomolecule is a biomolecule selected from the group consisting of: a natural isoform of reflectin protein, a non-natural variant of reflectin protein, a non-natural biomolecule derived from or mimicking reflectin protein, and any combination thereof; and wherein the living biological cell already comprises various cell components prior to transfection; wherein the living cell and its various cell components become individually characterized by a tunable refractive index and tunable optical properties.

40. The method of claim 39, wherein the tunable refractive index and the tunable optical properties are adjusted via one or more of the choices selected from the group consisting of: choice of the plasmid, choice of the reflectin biomolecule, the expression level of the reflectin biomolecule, and any combination thereof.


SEQUENCE LISTING (44 sequences)

SEQ ID NO: 1 - protein, 28 aa, artificial sequence
  Conserved Motif
  misc_feature (5)..(6): Xaa can be any naturally occurring amino acid
  misc_feature (8)..(8): Xaa can be any naturally occurring amino acid
  misc_feature (12)..(16): Xaa can be any naturally occurring amino acid
  misc_feature (19)..(28): Xaa can be any naturally occurring amino acid
MEPMXXMXMDFXXXXXDSXXXXXXXXXX

SEQ ID NO: 2 - protein, 28 aa, artificial sequence
  Conserved Motif
  misc_feature (4)..(5): Xaa can be any naturally occurring amino acid
  misc_feature (8)..(11): Xaa can be any naturally occurring amino acid
  misc_feature (14)..(15): Xaa can be any naturally occurring amino acid
  misc_feature (17)..(27): Xaa can be any naturally occurring amino acid
PERXXDMXXXXMDXXGXXXXXXXXXXXP

SEQ ID NO: 3 - protein, 21 aa, artificial sequence
  Conserved Motif
  misc_feature (1)..(1): Xaa can be any naturally occurring amino acid
  misc_feature (3)..(7): Xaa can be any naturally occurring amino acid
  misc_feature (10)..(14): Xaa can be any naturally occurring amino acid
  misc_feature (16)..(21): Xaa can be any naturally occurring amino acid
XDXXXXXMDXXXXXMXXXXXX

SEQ ID NO: 4 - protein, 357 aa, Doryteuthis pealeii
MAHHHHHHNRYLNRQRLYNMYRNKYRGVMEPMSRMTMDFQGRYMDSQGRMVDPRYYDYYGRMHDHDRYYGRSMFNQGHSMDSQRYGGWMDNPERYMDMSGYQMDMQGRWMDAQGRFNNPFGQMWHGRQGHYPGYMSSHSMYGRNMYNPYHSHYASRHFDSPERWMDMSGYQMDMQGRWMDNYGRYVNPFNHHMYGRNMCYPYGNHYYNRHMEHPERYMDMSGYQMDMQGRWMDTHGRHCNPFGQMWHNRHGYYPGHPHGRNMFQPERWMDMSGYQMDMQGRWMDNYGRYVNPFSHNYGRHMNYPGGHYNYHHGRYMNHPERHMDMSSYQMDMHGRWMDNQGRYIDNFDRNYYDYHMY

SEQ ID NO: 5 - protein, 350 aa, Doryteuthis pealeii
MNRYLNRQRLYNMYRNKYRGVMEPMSRMTMDFQGRYMDSQGRMVDPRYYDYYGRMHDHDRYYGRSMFNQGHSMDSQRYGGWMDNPERYMDMSGYQMDMQGRWMDAQGRFNNPFGQMWHGRQGHYPGYMSSHSMYGRNMYNPYHSHYASRHFDSPERWMDMSGYQMDMQGRWMDNYGRYVNPFNHHMYGRNMCYPYGNHYYNRHMEHPERYMDMSGYQMDMQGRWMDTHGRHCNPFGQMWHNRHGYYPGHPHGRNMFQPERWMDMSGYQMDMQGRWMDNYGRYVNPFSHNYGRHMNYPGGHYNYHHGRYMNHPERHMDMSSYQMDMHGRWMDNQGRYIDNFDRNYYDYHMY

SEQ ID NO: 6 - protein, 230 aa, Doryteuthis pealeii
MNRYMMRHRPMYSNMYRTGRKYRGVMEPMSRMTMDFQGRYMDSQGRMVDPRYYDYGRCHDYDRYYGRSMFNYGPNMDGQRYGGWMDFPERYMDMSGYQMDMHGRWMDSQGRYCNPMGHSWSNRQGYYPGSNYGRNMFNPERYMDMSGYQMDMQGRWMDMGGRHVNPFSHSMYGRNMFNPSYFSNRHMDNPERYMDMSGYQMDMQGRWMDTQGRYMDPSWSNMYDNYNSWY

SEQ ID NO: 7 - protein, 260 aa, Doryteuthis pealeii
MSSFMDPMHYDGMGMSHSKTGDFSHNCMRSFHKSQRDVMRRDIMGKSSKNRRFGNLMEPMSRMTMDFHGRLIDSQGRIVDPGHYFAMDDHYMENDRFLYPHDMLRNRHGMYGFMQGDYGNNMHRGMFADGMYRDMHHSGMNPSGYMHGGSMQNRPMMYMQGRYLDDSYFMNYHDPPVIVHSHYNDQEGRHHGMYDRHSDSYGSHRRHGDSHSMPRRPSESHSPQRRPSEGHIIQVRPEGGSSRKTSRAQLFPDDKLTDSA

SEQ ID NO: 8 - protein, 350 aa, Doryteuthis opalescens
MNRYLNRQRLYNMYRNKYRGVMEPMSRMTMDFQGRYMDSQGRMVDPRYYDHYGRMHDYDRYYGRSMFNQGHSMDSQRYGGWMDNPERYMDMSGYQMDMQGRWMDAQGRYNNPFSQMWHSRQGHYPGYMSHHSMYGRNMHYPYHSHSASRHFDSPERWMDMSGYQMDMQGRWMDNYGRYVNPFHHHMYGRNMFYPYGSHCNNRHMEHPERYMDMSGYQMDMQGRWMDTHGRHCNPLGQMWHNRHGYYPGHPHGRNMFQPERWMDMSSYQMDMQGRWMDNYGRYVNPFSHNYGRHMNYPGGHYNYHHGRYMNHPERQMDMSGYQMDMHGRWMDNQGRYIDNFDRNYYDYHMY

SEQ ID NO: 9 - protein, 230 aa, Doryteuthis opalescens
MNRYMMRHRPMYSNMYRTGRKYRGVMEPMSRMTMDFQGRYMDSQGRMVDPRYYEYGRCHDYDRYNGRSMFNNGPYMDGQRYGGWMDFPERYMDMSGYQMDMHGRWMDSQGRYCNPMGHSWSNRQGYYPGSNYGRNMFNPERYMDMSGYQMDMQGRWMDMGGRHVNPFSHSMYGRNMFNPSYFSNRHMDNPERYMDMSGYQMDMQGRWMDTQGRYMDPSMSNMYDNYNYWY

SEQ ID NO: 10 - protein, 150 aa, Doryteuthis opalescens
MSSFMDPMHYDGMGMSHSKSGDFSHNCMRSFHKSQRDGMRRDIMGKSSKNRRFGNLMEPMSRMTMDFHGRLIDSQGRIVDPGHYFAMDDHYMENDRFLYPHDMLRNRHGMYGFMQGDYGNNMHRGMFADGMYRDMHHSGMNPSSYMHGGS

SEQ ID NO: 11 - protein, 110 aa, Doryteuthis opalescens
MQNRPMMYMQGRYLDDSYFMNYHDPPVIVHSHYNDQEGRHQGMYDRHSDSYGSHRRHGDSHSMPRRPSESHSPQRRPSEGHIIQVRPEGGSSRKTSRAQLFPDDKLTDSA

SEQ ID NO: 12 - protein, 264 aa, Loligo forbesii
MNRSMNRYQPSNMWGNMNRDRYSGMMEPMSRMSMDFQGRHMDSMDRMVDPGRWNDYDRYYGRSTFNYGWMENGDRFNRNLRPMDFPERYMDMSDYQMDMGGRWMDPYGRQCNPFNQCGYNRHGYYPGYSYGRNMCYPERWMDMSNYSMDMQGRYMDRRGRHCNPFSQHTNWYGRYRNYPGDNNYYNRNMYYPERHFDMSNWQMDMQGRWMDNQGRYNNPYWYGRNMYQPYQNNQWSGRWDYPGMDCGMDMQGGYMNNSNEGDYL

SEQ ID NO: 13 - protein, 242 aa, Sepia officinalis
MNRSMNRWRPMFNNMHNNYYGRSMFNYDWMMDGDRYNRYYRWMDFPERYMDMSGYQMDMYGRWMDMQGQHYNPFRQWGYQRHGYYPGYHYGSNMFYPERWMDMSNYSMDMQGRYMDRWGRHCNPFSHYYNHWNRYWNHPGYYNNHYNRHMYYPERYFDMSNWQMDMQGRWMDMQGRHCNPYWYNWHGRHMYYPYQNYYWYGRWDYPGMDYSNWQMDFQGRWMDNHGRHMNPWWNEHYFNHYY

SEQ ID NO: 14 - protein, 242 aa, Sepia officinalis
MNRSMNRWRPMFNNMHNNYYGRSMFNYDWMMDGDRYNRYYRWMDFPERYMDMSGYQMDMYGRWMDMQGQHYNPFRQWGYQRHGYYPGYHYGSNMFYPERWMDMSNYSMDMQGRYMDRWGRHCNPFSHYYNHWNRYWNHPGYYNNHYNRHMYYPERYFDMSNWQMDMQGRWMDMQGRHCNPYWYNWHGRHMYYPYQNYYWYGRWDYPGMDYSNWQMDFQGRWMDNHGRHMDPWWNEQYFNHYY

SEQ ID NO: 15 - protein, 242 aa, Sepia officinalis
MNRFMNRYRPMFNNMHNNYYGRSMFNYDWMMDGDRYNRYYRWMDFPERYMDMSGYQMDMYGRWMDMQGHHCNPFRQWGYQRHGYYPGFHYGSNMFYPERWMDMSNYSMDMQGRYMDRWGRHCNPFSHYYNHWNRYWNHPGYYNYHYNRHMYYPERYFDMSNWQMDMQGRWMDMQGRHSNPYWYNWHGRHMYYPYQNYYWYGRWDHHGMDYSNWQMDFQGRWMDNHGRHMNPWWNEQYFNHYY

SEQ ID NO: 16 - protein, 242 aa, Sepia officinalis
MNRFMNRYRPMFNNMHNNYYGRSMFNYDWMMDGDRYNRYYRWMDFPERYMDMSGYQMDMYGRWMDMQGRHCNPFRQWGYQRHGYYPGFHYGSNMFYPERWMDMSNYSMDMQGRYMDRWGRHCNPFSQYYNHWNRYWNHPGYYNNHYNRHMYYPERYFDMSNWQMDMQGRWMDMQGRHSNPYWYNWHGRHMYYPYQNYYWYGRWDHHGMDYSNWQMDFQGRWMDNHGRHMDPWWNEQYFNHYY

SEQ ID NO: 17 - protein, 242 aa, Sepia officinalis
MNRFMNRYRPMFNNMHNNYYGRSMFNYDWMMDGDRYNRYNRWMDFPERYMDMSGYQMDMYGRWMDMQRHHCNPFRQWGYQRHGYYPGFHYGSNMFYPERWMDMSNYSMDMQGRYMDRWGRHCNPFSHYYNHWNRYWNHPGYYNNHYNRHMYYPERYFDMSNWQMDMQGRWMDMQGRHSNPYWYNWHGRHMYYPYQNYYWYGRWDHHGMDYSNWQMDFQGRWMDNHGHHMNPWWNEQYFNHYY

SEQ ID NO: 18 - protein, 239 aa, Sepia officinalis
MNRSMNRWRPMFNNMHNNYYGRSMFNYNWMMDGDRYNRYYRWMDFPEWYMDMSGYQMDMYGRWMDMHGRHCNPFRQWGYQRHGYYPGFHYGSNMFYPERWMDMSNYSMDMQGRYMDRWGRHCNPFSHYYNHWNRYWNHPGYYNYYYMYYPERCYDMSNWQMDMQGRWMDMQGRHSNPYWYNWHGRHMYYPYQNYYWYGRWDHHGMDYSNWQMDFQGRWMDNHGRHMDPWWNEHYFNHYY

SEQ ID NO: 19 - protein, 239 aa, Sepia officinalis
MNRFMNRYRPMFNNMHNNYYGRSMFNYNWMMDGDRYNRYYRWMDFPERYMDMSGYQMDMYGRWMDMQGRHCNPFRQWGYQRHGYYPGFHYGSNMFYPERWMDMSNYSMDMQGRYMDRWGRHCNPFSYYYNHWNRYWNHPGYYNYYYMYYPERYYDMSNWQMDMQGRWMDMQGRHSNPYWYNWHGRHMYYPYQNYYWYGRWDYPGMDYSNWQMDFQGRWMDNHGRHMNPWWNEQYFNHYY

SEQ ID NO: 20 - protein, 239 aa, Sepia officinalis
MNRSMNRWRPMFNNMHNNYYGRSMFNYNWMMDGDRYNRYYRWMDFPEWYMDMSGYQMDMYGRWMDMHGRHCNPFRQWGYQRHGYYPGFHYGSNMFYPERWMDMSNYSMDMQGRYMDRWGRHCNPFSHYYNHWNRYWNHPGYYNYYYMYYPERYYDMSNWQMDMQGRWMDMQGRHSNPYWYNWHGRHMYYPYQNYYWYGRWDHHGMDYSNWQMDFQGRWMDNHGRHMDPWWNEHYFNHYY

SEQ ID NO: 21 - protein, 290 aa, Sepia officinalis
MNRFMNRWRPMFNNMRNNMYRGRYRGMMEPMSRMTMDFQGRYMDSQGRMVDPRYYDYYGRWNDYDRYYGRSMFNYSWMMDGDRYNRYYRWMDFPERYMDMSGYQMDMYGRWMDMHGRHCNPFRQWGYQRHGYYPGFHYGSNMFYPERWMDMSHYSMDMQGRYMDRWGRHCNPFSHYYNHWNRYWNHPGYYNNHYNRHMYYPERYFDMSNWQMDMQGRWMDMQGRHSDPYWYNWHGRHMYYPYQNYYWYGRWDHHGMDYSNWQMDFQGRWMDNHGRHMDPWWNEHYFNHYY

SEQ ID NO: 22 - protein, 290 aa, Sepia officinalis
MNRFMNRWRPMFNNMRNNMYRGRYRGMMEPMSRMTMDFQGRYMDSQGRMVDPRYYDYYGRWNDYDRYYGRSMFNYSWMMDGDRYNRYYRWMDFPERYMDMSGYQMDMYGRWMDMHGRHCNPFRQWGYQRHGYYPGFHYGSNMFYPERWMDMSHYSMDMQGRYMDRWGRHCNPFSHYYNHWNRYWNHPGYYNNHYNRHMYYPERYFDMSNWQMDMQGRWMDMQGRHSNPYWYNWHGRHMYYPYQNYYWYGRWDHHGMDYSNWQMDFQGRWMDNHGRHMDPWWNEHYFNHYY

SEQ ID NO: 23 - protein, 290 aa, Sepia officinalis
MNRFMNRWRPMFNNMHNNMYRGRYRGMMEPMSRMTMDFQGRYMDSQGRMVDPRYYDYYGRWNDYDRYYGRPMFNYSWMMDGDRYNRNYRWMDFPERYMDMSGYQMDMYGRWMDMQGHHCNPFRQWGYQRHGYYPGFHYGSNMFYPERWMDMSNYSMDMQGRYMDRWGRHCNPFSHYYNHWNRYWNHPGYYNNHYNRHMYYPERYFDMSNWQMDMQGRWMDMQGRHSNPYWYNWHGRHMYYPYQNYYWYGRWDHPGMDYSNWQMDFQGRWMDNHGRHMNPWWNEHYFNHYY

SEQ ID NO: 24 - protein, 283 aa, Sepia officinalis
MNRYMNRFRNWYGNNYRGRYRGMMEPMSRMTMDFQGRYMDSQGRMVDPRYYDYYGRWNDYDRYYGRPMFNYSWMMDGDRYNRYYRWMDFPERYMDMSGYQMDMYGRWMDMQGRHCNPFRQWGYQRHGYYPGYHYGSNMFYPERWMDMSNYSMDMQGRYMDRWGRHCNPFSHYYNHWNRYWNHPGYYNYYYMYYPERYYDMSNWQMDMQGRWMDMQGRHCNPYWYNWHGRHMYYPYQNYYWYGRWDYPGMDYSNWQMDFQGRWMDNQGRYMDPWWWNDYYSYYY

SEQ ID NO: 25 - protein, 284 aa, Sepia officinalis
MNRYMNRFRNWYGNNYRGRYRGMMEPMSRMTMDFQGRYMDSQGRMVDPRYYDYYGRWNDYDRYYGRSMFNYSWMMDGDRYNRYYRWMDFPERYMDMSGYQMDMYGRWMDMQGRHCNPFRQWWYHRHGYYPGFHYGSNMFYPERWMDMSNYSMDMQGRYMDRWGRHCNPFSHYYNHWNRYWNHPGYYNYYYMYYPERYYDMSNWQMDMQGRWMDMQGRHSNPYWYNWHGRHMYYPYQNYYWYGRWDYPGMDYSNWQMDFQGRWMDNQGRYMDPWWWNDYYYNYYY

SEQ ID NO: 26 - protein, 284 aa, Sepia officinalis
MNRYTMRNRPMYGNMYRTGKKYRGVMEPMSRMTMDFQGRYMDSQGRMVDPRHNDYYGRWNDYDRYYGRSMFNYGPHMDGHQHGGWMDFPERWMDMSNYQMDMQGRWMDMQGRHCQPFNQWGYNRHGNYPSSYYGRNMFYPERWMDMSNWQMDTQGRWMDMQGRYGSPFNQWGYNRHGYYPGSSYGRNMYHPERWMDMSNYQMDMQGRWMDMHGRHVNPFSHSMHGRNWSYPYYNYYSSRHMDYPERNMDMSNWQMDMQGRWMDMQGRHMDPSWSNMHDNHNYWF

SEQ ID NO: 27 - protein, 284 aa, Sepia officinalis
MNRYMMRNRPMYGNMYRTGKKYRGVMEPMSRMTMDFQGRYMDSQGRMVDPRHNDYYGRWNDYDRYYGRSMFNYGPHMDGHQHGGWMDFPERWMDMSNYQMDMQGRWMDMQGRHCQPFNQWGYNRHGNYPSSYYGRNMFYPERWMDMSNWQMDTQGRWMDMQGRYGSPFNQWGYNRHGYYPGSSYGRNMYHPERWMDMSNYQMDMQGRWMDMHGRHVNPFSHSMHGRNWSYPYYNYYSSRHMDYPERNMDMSNWQMDMQGRWMDMQGRHMDPSWSNMHDNHNYWF

SEQ ID NO: 28 - protein, 291 aa, Sepia officinalis
MNRFMNRYRPMFNNMHNNMYNNMYRGRYRGMMEPMSRMTMDFQGRYMDSQGRMVDPRYYDYYGRWNDYDRYYGKSMFNYGWMMDGDRYNNYYRWMDFPERYMDMSGYQMDMYGRWMDMQGRHCNPFNQWGHNRYGQSFNYNYGRNMFYPERWMDMSNYSMDMQGRYMDRWGRHCNPFSQNMNWYGRYWNYPGYNNYYYNRHMYYPERYFDMSNWQMDMQGRWMDMQGRHNNPYWYNWYGRQMYYPYQNNWYGRWDYPGMDYSNWQMDMQGRWMDMQGRYMDPWMSDYSYNN

SEQ ID NO: 29 - protein, 267 aa, Sepia officinalis
MSTFMDPMFYEGLGMPPPNFGDFNHNCMRSFHKSQRDMMRRDIMAKSSKNKRCGDLMEPMSRMIMDFNGGLIDSRRRITDSDHYFTIDGNYGDNEKPLTSDGLLRNRYDMYAFAPADKCHNRTRGLYGDSMYRDKHQDGMYSSGYMQGRSMQNHRMMGSFQGGMQTQSRYMDDPYYVNYNSGTYDTPVDMNSYYFDQEGRHRMYSRFSEGQITPGRQEGTYSARRESRSGQRRLSDSHSFQRPIDTRSNRRTSHGMLYSERNNIDFA

SEQ ID NO: 30 - protein, 283 aa, Euprymna scolopes
MNRFMNRYRPMFNNMYSNMYRGRYRGMMEPMSRMTMDFQGRYMDSQGRMVDPRYYDYYGRFNDYDRYYGRSMFNYGWMMDGDRYNRYNRWMDYPERYMDMSGYQMDMSGRWMDMQGRHCNPYSQWMMYNYNRHGYYPNYSYGRHMFYPERWMDMSNYSMDMYGRYMDRWGRYCNPFSQYMNYYGRYWNYPGYNNYYYSRNMYYPERYFDMSNWQMDMQGRWMDNQGRYCSPYWNNWYGRHMYYPYQNNYFYGRYDYPGMDYSNYQMDMQGRYMDQYGMNDYYY

SEQ ID NO: 31 - protein, 282 aa, Euprymna scolopes
MNRFMNKYRPMFNNMYSNMYRGRNRGMMEPMSRMTMDFQGRYMDSQGRMVDPRYYDYYGRFNDYDRYYGRSMFNYGWMMDGDRYNRYNRWMDYPERYMDMSGYQMDMSGRWMDMQGRHCNPYSQWGYNYNRHGYYPNYSYGRHMFYPERWMDMSGYQMDMQGRYMDRWGRYCNPFSQYMNYYGRYWNYPGYNSYYNSRNMFYPERYFDMSNWQMDMQGRWMDNQGRYCSPYWNNWYGRQMYYPYQNNYFYGRYDYPGMDYSNYQMDMQGRYMDQYGMNDYCY

SEQ ID NO: 32 - protein, 283 aa, Euprymna scolopes
MNRYMTRFRNFYGNMYRGRYRGMMEPMSRMTMDFQGRYMDSQGRMVDPRYYDYYGRYNDYDRYYGRSMFNYGWMMDGDRYNRYNRWMDFPERYMDMSGYQMDMYGRWMDMQGRHCNPYSQWMMYNYNRHGYYPNYSYGRHMFYPERWMDMSNYSMDMYGRYMDRWGRYCNPFYQFYNHWNRYGNYPGYYNYYYMYYPERYFDMSNWQMDMQGRWMDMQGRYCSPYWYNWYGRHMYYPYQNYYWYGRYDYPGMDYSNWQMDMQGRWMDMQGRYMDYPYNYYNWY

SEQ ID NO: 33 - protein, 284 aa, Euprymna scolopes
MNRYMNRFRNFYGNMYRGRYRGMMEPMSRMTMDFQGRYMDSQGRMVDPRFYDYYGRYNDYDRYYGRSMFNYGWMMDGDRYNRCNRWMDYPERYMDMSGYQMDMYGRWMDMQGRHCNPYSQWMMYNYNRHGYYPNYSYGRHMFYPERWMDMSNYSMDMYGRYMDRWGRYCNPFYQFYNHWNRYGNYPGYYNYYYMYYPERYFGMSNWQMDMQGRWMDMQGRYCSPYWYNWYGRHMYYPYQNYYWYGRYDYPGMDYSNWQMDMQGRWMDMQGRYMDYPYNYYNWNH

SEQ ID NO: 34 - protein, 284 aa, Euprymna scolopes
MNRYMNRFRNFYGNMYRGRYRGMMEPMSRMTMDFQGRYMDSQGRMVDPRYYDYYGRYNDYDRYYGRSMFNYGWMMDGDRYNRYNRWMDFPERYMDMSGYQMDMYGRWMDMQGRHCNPYSQWMMYNYNRHGYYPNYSYGRHMFYPERWMDMSNYSMDMYGRYMDRWGRYCNPFYQFYNHWNRYGNYPGYYNYYYMYYPERYFDMSNWQMDMQGRWMDMQGRYCSPYWYNWYGRQMYYPYQNYYWYGRYDYPGMDYSNYQMDMQGRYMDMQGRYMDYPYNYYNWNH

SEQ ID NO: 35 - protein, 284 aa, Euprymna scolopes
MNRYMNRFRNFYGNMYRGRYRGMMEPMSRMTMDFQGRYMDSQGRMVDPRYYDYYGRFNDYDRYYGRSMFNYGWMMDGDRYNRYNRWMDFPERYMDMSGYQMDMYGRWMDMQGRHCNPYSQWMMYNYNRHGYYPNYSYGRHMFYPERWMDMSNYSMDMYGRYMDRWGRYCNPFYQFYNHWNRYGNYPGYYSYYYMYYPERYFDMSNWQMDMQGRWMDMQGRYCSPYWYNWYGRHMYYPYQNYYWYGRYDYPGMDYSNWQMDMQGRWMDMQGRYMDYPYNYYNWNH

SEQ ID NO: 36 - protein, 288 aa, Euprymna scolopes
MNRYMNRFRNFYGNMCRNRNRGMMEPMSRMTMDFQGRYMDSQGRMVDPRYYDYYGRYNDYDRYYGRSMFNYGWMMDGDRYNRYNRWMDYPERYMDMSGYQMDMYGRWMDMQGRHCNPYSQWMMYNYNRHGYYPNYSYGRHMFYPERWMDMSNYSMDMYGRYMDRWGRYCNPFYHYYNHWNRSGNNPGYYSYYYMYYPERYFDMSNWQMDMQGRWMDMQGRYCSPYWYNWYGRQMYYPYQNYYWYGRWDYPGMDYSNWQMDMQGRWMDMQGRYMDPWWMNDSYYNNYYN

SEQ ID NO: 37 - protein, 95 aa, Euprymna scolopes
MYYPERYFDMSNWQMDMQGRWMDMQGRYCSPYWYNWYGRQMYYPYQNYYWYGRWDYPGMDYSNWQMDMQGRWMDMQGRYMDPWWMNDSYYNNYYN

SEQ ID NO: 38 - protein, 442 aa, Octopus bimaculoides
MNRSRNMFRNSSRKHRGVMEPMTRMTMDFQGRYLDSSGRLVEPRCNDYYGRNSNYDRYRPMQNTGIYDNDKFQKYGRFMHFPERQMDMSGYQMDMRGRYMDKYGRHCNPYSRRHMNYPNNNYDNYHMYNPEKLMDMSNFQMDMHGRWMDSNGRYSSPFSNYGSRHHQNYPHFNYNWGQRGFNYPDRFFDMSNYQMDLDGKWMDTYGRHCHPFYDNSNYYGKQYNYNMYPHYNYNWGQKYYHYPERYFDMSNYQMDFDGRWMDMFGRSHSPFNGYNNNQGRQHHGQPHNSFSYGQRYQDRNFDIGNYQMDFDGRWMDMYGRYSHPFYGYNNFQSRYQHNLPQNFNWGQRSFHNPERLFDMGNYQMDFDGHWMDMDDRHCQPFTGNYNHSNRYQQNCNHSPSQNFNWSQRYQDNPEKFFDMSGYQMEFDGRWMDSNNYNSDNFW

SEQ ID NO: 39 - protein, 99 aa, Octopus bimaculoides
MYGHIYNGRMEPMHMMSMDFHGRYMDSYGRMVDPRSYGFYGRYHDQDSYYGRSMYNNHNFHDFDRFHRFDYFMNFPDMFMDMSGYEMDFNGRWMNMHRF

SEQ ID NO: 40 - protein, 115 aa, Octopus bimaculoides
HPFHSFSGYHGYHHSGYYPYHSYSQGRRYHNYYDMFYDMSHYQMDFDGNWMDMYNHYSHPFFGYNHYHRGSHYYNHYPYHNYSWGHRFYDYPERFFDMSHYEMDFNGRWMNMHRF

SEQ ID NO: 41 - protein, 81 aa, Octopus bimaculoides
MFYDMSHYQMDFDGNWMDMYNHYSHPFFGYDHYHRGSHYYNHYPYHNYSWGHRFYDYPERFFDMSHYEMDFNGRWMNMHRF

SEQ ID NO: 42 - protein, 365 aa, Octopus bimaculoides
MNRLMNKFRHHFGRKYRGIMEPMSVMSMDFQGRYMDSYGRMVDPRFYEFYGRYSDNDRYYGKSMYNYYGFYDNDRFHRYGNFMDFPERFMDMSSYQMDMYGRWMDMHGHHSSPYWYMFNSSRHGHYPGYRYGRNWFYPERFMDMSHYQMDMYGRYMDRYGRQCNPYYNYYRRYMYYPYMNFYYMHYPERFMDMSGYQMDMYGRWMDMYGRHSTPFYTNYGRYYHNYPYYNYSWGQRYYNYPERYFDMTNYQMDFDGRWMDMYSRHCTPFYSYHGRYHHYYPYHSYSWGQRFYNNPERWYDMDYEFHSMSPYNYHSRYHYFNYSPYFSGGHRWFDMSNYQMDFGGQWMDMNGRYMNHFDHWNEYFF

SEQ ID NO: 43 - protein, 483 aa, Octopus bimaculoides
MNRYMNRRNNFSRRYRGIMEPMSRMTMDFQGRYMDSYGRMVDPRFYGFYGRYSDNDRYYGRSMYNYYGFYDNDRFHRYGNFMDFPERFMDMSGYQMDMSGRWMDMHGHYSSPHWHMFNSSRQGYYPGYHYGRNWFYPERFMDMSHYQMDMYGRYMDRNGRHCNPYYNYYRRYMYYPYMNFYHMYYPERFMDMSGYQMDMYGRWMDMSGRHSSPFYSYHSRFHHNYPYYNYSWGQRYYNYPERYFDMGNYQMDFDGRWMDMYSRHCTPFYNYHGKFHHNYPYYNYSWGQRYYNYPERYFDMGNYQMDFDGRWMDNYGRYSSPFNSYHGRFHHNYPYYNYSWGQRYYNYPERYFDMGNYQMDFDGRWMDNYGRYSSPFYNYHGRYHNYPYYNYSWGQRYYNYPEGNYQMDFDGRWMDNYGRYFHGYNYHNRHYYNSYPNSYNYNWGQRYYDYPERNFDMFNYQMDFDSRWMDGQNFHYYGDNYNY

SEQ ID NO: 44 - protein, 344 aa, Octopus bimaculoides
MNRFMNRFRPQFNRKYRGFMEPMNMMSMVFQGRYMDSYGKMVDPKLYEFYGKYSDNDRYYGKSMYNYYGFYDNDRFHRNGNFMDFPERFMDMSGYQMDMNGKWMDTQGQNSHPYWNMFSSSRQGCYPGYSYGRNWFFPERFMDMSHYQMDMNGRYMDKSGRHCNPYYSYYRRYMSHPQMNFNQMHYPERFMDMSSYQMDIGGRYMDKWGCHINPFSTYYFGKQSYFPHNYWSQRKYMDMSSYQMDMQYNSMDMNSRNCDQLHYFRNFDMWNNQMDFDGHWMNMNNQSYHPSSFIRNQVYYNPYHFYTWMSRYYNHPEKFYDTSNYQVEFGGKWPSQEYECITQE